SEQUENCE LISTING (73 sequences)

SEQ ID NO: 1 - DNA, 3684 bp, unidentified
  source 1..3684: mol_type = other DNA; note = Lachnospiraceae bacterium
atgagcaaactggaaaagttcaccaactgttatagcctgagcaaaaccctgcgttttaaagcaattccggttggtaaaacccaagagaacattgataataaacgcctgctggtcgaagatgaaaaacgcgctgaagattataaaggcgtgaaaaaactgctggatcgctattatctgagcttcattaacgatgtgctgcacagcattaaactgaagaacctgaacaactatatcagcctgtttcgtaaaaaaacccgcaccgaaaaagaaaacaaagagctggaaaacctggaaatcaatctgcgtaaagaaatcgccaaagcgtttaaaggtaacgagggttataaaagcctgttcaagaaagacatcatcgaaaccattctgccggaatttctggatgataaagatgaaattgccctggtgaatagctttaatggctttaccaccgcatttaccggcttttttgataatcgcgaaaacatgttcagcgaagaagcaaaaagcaccagcattgcatttcgctgcattaatgaaaatctgacccgctacattagcaacatggatatctttgaaaaagtggacgcgatcttcgataaacacgaagtgcaagagatcaaagagaaaatcctgaacagcgattatgacgtcgaagatttttttgaaggcgagttctttaacttcgttctgacccaagaaggtatcgacgtttataacgcaattattggtggttttgttaccgaaagcggtgagaaaatcaaaggcctgaatgaatatatcaacctgtataaccagaaaaccaaacagaaactgccgaaattcaaaccgctgtataaacaggttctgagcgatcgtgaaagcctgagcttttatggtgaaggttataccagtgatgaagaggttctggaagtttttcgtaacaccctgaataaaaacagcgagatctttagcagcatcaaaaagcttgagaaactgttcaaaaactttgatgagtatagcagcgcaggcatctttgttaaaaatggtccggcaattagcaccatcagcaaagatatttttggcgaatggaatgtgatccgcgataaatggaatgccgaatatgatgatatccacctgaaaaaaaaggccgtggtgaccgagaaatatgaagatgatcgtcgtaaaagcttcaagaaaattggtagctttagcctggaacagctgcaagaatatgcagatgcagatctgagcgttgtggaaaaactgaaagaaatcatcattcagaaggtggacgagatctataaagtttatggtagcagcgaaaaactgttcgatgcagattttgttctggaaaaaagcctgaaaaagaatgatgccgttgtggccattatgaaagatctgctggatagcgttaagagcttcgagaattacatcaaagccttttttggtgagggcaaagaaaccaatcgtgatgaaagtttctatggcgattttgtgctggcctatgatattctgctgaaagtggaccatatttatgatgccattcgcaattatgttacccagaaaccgtatagcaaagacaagttcaaactgtactttcagaacccgcagtttatgggtggttgggataaagataaagaaaccgattatcgtgccaccatcctgcgttatggtagtaaatactatctggccatcatggacaaaaaatacgcaaaatgcctgcagaaaatcgacaaagatgatgtgaatggcaactatgaaaaaatcaactacaaactgctgcctggtccgaataaaatgctgccgaaagtgttctttagcaagaaatggatggcctattataacccgagcgaggatattcaaaagatctacaaaaatggcacctttaaaaagggcgacatgttcaatctgaacgattgccacaaactgatcgatttcttcaaagattcaatttcgcgttatccgaaatggtccaatgcctatgattttaactttagcgaaaccgaaaaatacaaagacattgccggtttttatcgcgaagtggaagaacagggctataaagtgagctttgaaagcgcaagcaaaaaagaggttgataagctggttgaagagggcaaactgtatatgttccagatttacaacaaagattttagcgacaaaagccatggcaccccgaatctgcataccatgtactttaaactgctgttcgacgaaaataaccatggtcagattcgtctgagcggtggtgccgaactgtttatgcgtcgtgcaagtctgaaaaaagaagaactggttgttcatccggcaaatagcccgattgcaaacaaaaatccggacaatccgaaaaaaaccacgacactgagctatgatgtgtataaagacaaacgttttagcgaggatcagtatgaactgcatatcccgattgccatcaataaatgcccgaaaaacatctttaagatcaacaccgaagttcgcgtgctgctgaaacatgatgataatccgtatgtgattggcattgatcgtggtgaacgtaacctgctgtatattgttgttgttgatggtaaaggcaacatcgtggaacagtatagtctgaacgaaattatcaacaactttaacggcatccgcatcaaaaccgactatcatagcctgctggacaagaaagaaaaagaacgttttgaagcacgtcagaactggaccagtattgaaaacatcaaagaactgaaagccggttatattagccaggtggttcataaaatctgtgagctggtagaaaaatacgatgcagttattgcactggaagatctgaatagcggtttcaaaaatagccgtgtgaaagtcgaaaaacaggtgtatcagaaattcgagaaaatgctgatcgacaaactgaactacatggtcgacaaaaaaagcaatccgtgtgcaaccggtggtgcactgaaaggttatcagattaccaacaaatttgaaagctttaaaagcatgagcacccagaacggctttatcttctatattccggcatggctgaccagcaaaattgatccgagcaccggttttgtgaacctgctgaaaacaaaatatacctccattgccgacagcaagaagtttattagcagctttgatcgcattatgtatgttccggaagaggacctgtttgaattcgcactggattacaaaaatttcagccgtaccgatgccgactacatcaaaaaatggaaactgtacagctatggtaaccgcattcgcatttttcgcaacccgaagaaaaacaatgtgttcgattgggaagaagtttgtctgaccagcgcatataaagaacttttcaacaaatacggcatcaactatcagcagggtgatattcgtgcactgctgtgtgaacagagcgataaagcgttttatagcagttttatggcactgatgagcctgatgctgcagatgcgtaatagcattaccggtcgcaccgatgtggattttctgattagtccggtgaaaaattccgatggcatcttttatgatagccgcaattacgaagcacaagaaaatgcaattctgccgaaaaacgcagatgcaaatggtgcatataacattgcacgtaaagttctgtgggcaattggccagtttaagaaagcagaagatgagaagctggacaaagtgaaaattgcgatcagcaataaagagtggctggaatacgcacagaccagcgttaaacat

SEQ ID NO: 2 - protein, 1186 aa, unidentified
  source 1..1186: note = Lachnospiraceae bacterium
MSKLEKFTNCYSLSKTLFKAIPVGKTQENIDNKLLVEDEKAEDYKGVKKLLDYYLSFINDVLHSIKLKNLNNYISLFKKTTEKENKELENLEINLKEIAKAFKGNEGYKSLFKKDIIETILPEFLDDKDEIALVNSFNGFTTAFTGFFDNENMFSEEAKSTSIAFCINENLTYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDESLSFYGEGYTSDEEVLEVFNTLNKNSEIFSSIKKLEKLFKNFDEYSSAGIFVKNGPAISTISKDIFGEWNVIDKWNAEYDDIHLKKKAVVTEKYEDDKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNDESFYGDFVLAYDILLKVDHIYDAINYVTQKPYSKDKFKLYFQNPQFMGGWDKDKETDYATILYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISYPKWSNAYDFNFSETEKYKDIAGFYEVEEQGYKVSFESASKKEVDKLVEEGKLYMFQIYNKDFSDKSHGTPNLHTMYFKLLFDENNHGQILSGGAELFMASLKKEELVVHPANSPIANKNPDNPKKTTTLSYDVYKDKFSEDQYELHIPIAINKCPKNIFKINTEVVLLKHDDNPYVIGIDGENLLYIVVVDGKGNIVEQYSLNEIINNFNGIIKTDYHSLLDKKEKEFEAQNWTSIENIKELKAGYISQVVHKICELVEKYDAVIALEDLNSGFKNSVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDIMYVPEEDLFEFALDYKNFSTDADYIKKWKLYSYGNIIFNPKKNNVFDWEEVCLTSAYKELFNKYGINYQQGDIALLCEQSDKAFYSSFMALMSLMLQMNSITGTDVDFLISPVKNSDGIFYDSNYEAQENAILPKNADANGAYNIAKVLWAIGQFKKAEDEKLDKVKIAISNKEWLEYAQTSVKH

SEQ ID NO: 3 - protein, 1185 aa, synthetic construct
MSKLEKFTNCYSLSKTLFKAIPVGKTQENIDNKLLVEDEKAEDYKGVKKLLDYYLSFINDVLHSIKLKNLNNYISLFKKTTEKENKELENLEINLKEIAKAFKGNEGYKSLFKKDIIETILPEFLDDKDEIALVNSFNGFTTAFTGFFDNENMFSEEAKSTSIAFCINENLTYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDESLSFYGEGYTSDEEVLEVFNTLNKNSEIFSSIKKLEKLFKNFDEYSSAGIFVKNGPAISTISKDIFGEWNVIDKWNAEYDDIHLKKKAVVTEKYEDDKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNDESFYGDFVLAYDILLKVDHIYDAINYVTQKPYSKDKFKLYFQPQFMGGWDKDKETDYATILYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISYPKWSNAYDFNFSETEKYKDIAGFYEVEEQGYKVSFESASKKEVDKLVEEGKLYMFQIYNKDFSDKSHGTPNLHTMYFKLLFDENNHGQILSGGAELFMASLKKEELVVHPANSPIANKNPDNPKKTTTLSYDVYKDKFSEDQYELHIPIAINKCPKNIFKINTEVVLLKHDDNPYVIGIDGENLLYIVVVDGKGNIVEQYSLNEIINNFNGIIKTDYHSLLDKKEKEFEAQNWTSIENIKELKAGYISQVVHKICELVEKYDAVIALEDLNSGFKNSVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDIMYVPEEDLFEFALDYKNFSTDADYIKKWKLYSYGNIIFNPKKNNVFDWEEVCLTSAYKELFNKYGINYQQGDIALLCEQSDKAFYSSFMALMSLMLQMNSITGTDVDFLISPVKNSDGIFYDSNYEAQENAILPKNADANGAYNIAKVLWAIGQFKKAEDEKLDKVKIAISNKEWLEYAQTSVKH

SEQ ID NO: 4 - protein, 1186 aa, synthetic construct
MSKLEKFTNCYSLSKTLFKAIPVGKTQENIDNKLLVEDEKAEDYKGVKKLLDYYLSFINDVLHSIKLKNLNNYISLFKKTTEKENKELENLEINLKEIAKAFKGNEGYKSLFKKDIIETILPEFLDDKDEIALVNSFNGFTTAFTGFFDNENMFSEEAKSTSIAFCINENLTYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDESLSFYGEGYTSDEEVLEVFNTLNKNSEIFSSIKKLEKLFKNFDEYSSAGIFVKNGPAISTISKDIFGEWNVIDKWNAEYDDIHLKKKAVVTEKYEDDKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNDESFYGDFVLAYDILLKVDHIYDAINYVTQKPYSKDKFKLYFQNPQFMGGWDKDKETDYATILYGSKYYLAIMPKKYAKCLQKIDKDDVNGNYEKINYKLLPGPNKMLPKVFFSKKWMAYYNPSEDIQKIYKNGTFKKGDMFNLNDCHKLIDFFKDSISYPKWSNAYDFNFSETEKYKDIAGFYEVEEQGYKVSFESASKKEVDKLVEEGKLYMFQIYNKDFSDKSHGTPNLHTMYFKLLFDENNHGQILSGGAELFMASLKKEELVVHPANSPIANKNPDNPKKTTTLSYDVYKDKFSEDQYELHIPIAINKCPKNIFKINTEVVLLKHDDNPYVIGIDGENLLYIVVVDGKGNIVEQYSLNEIINNFNGIIKTDYHSLLDKKEKEFEAQNWTSIENIKELKAGYISQVVHKICELVEKYDAVIALEDLNSGFKNSVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDIMYVPEEDLFEFALDYKNFSTDADYIKKWKLYSYGNIIFNPKKNNVFDWEEVCLTSAYKELFNKYGINYQQGDIALLCEQSDKAFYSSFMALMSLMLQMNSITGTDVDFLISPVKNSDGIFYDSNYEAQENAILPKNADANGAYNIAKVLWAIGQFKKAEDEKLDKVKIAISNKEWLEYAQTSVKH

SEQ ID NO: 5 - protein, 1186 aa, synthetic construct
MSKLEKFTNCYSLSKTLFKAIPVGKTQENIDNKLLVEDEKAEDYKGVKKLLDYYLSFINDVLHSIKLKNLNNYISLFKKTTEKENKELENLEINLKEIAKAFKGNEGYKSLFKKDIIETILPEFLDDKDEIALVNSFNGFTTAFTGFFDNENMFSEEAKSTSIAFCINENLTYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDESLSFYGEGYTSDEEVLEVFNTLNKNSEIFSSIKKLEKLFKNFDEYSSAGIFVKNGPAISTISKDIFGEWNVIDKWNAEYDDIHLKKKAVVTEKYEDDKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNDESFYGDFVLAYDILLKVDHIYDAINYVTQKPYSKDKFKLYFQNPQFMGGWDKDKETDYATILYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISYPKWSNAYDFNFSETEKYKDIAGFYEVEEQGYKVSFESASKKEVDKLVEEGKLYMFQIYNKDFSDKSHGTPNLHTMYFKLLFDENNHGQILSGGAELFMASLKKEELVVHPANSPIANKNPDNPKKTTTLSYDVYKDKFSEDQYLLHIPIAINKCPKNIFKINTEVVLLKHDDNPYVIGIDGENLLYIVVVDGKGNIVEQYSLNEIINNFNGIIKTDYHSLLDKKEKEFEAQNWTSIENIKELKAGYISQVVHKICELVEKYDAVIALEDLNSGFKNSVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDIMYVPEEDLFEFALDYKNFSTDADYIKKWKLYSYGNIIFNPKKNNVFDWEEVCLTSAYKELFNKYGINYQQGDIALLCEQSDKAFYSSFMALMSLMLQMNSITGTDVDFLISPVKNSDGIFYDSNYEAQENAILPKNADANGAYNIAKVLWAIGQFKKAEDEKLDKVKIAISNKEWLEYAQTSVKH

SEQ ID NO: 6 - protein, 1185 aa, synthetic construct
MSKLEKFTNCYSLSKTLFKAIPVGKTQENIDNKLLVEDEKAEDYKGVKKLLDYYLSFINDVLHSIKLKNLNNYISLFKKTTEKENKELENLEINLKEIAKAFKGNEGYKSLFKKDIIETILPEFLDDKDEIALVNSFNGFTTAFTGFFDNENMFSEEAKSTSIAFCINENLTYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDESLSFYGEGYTSDEEVLEVFNTLNKNSEIFSSIKKLEKLFKNFDEYSSAGIFVKNGPAISTISKDIFGEWNVIDKWNAEYDDIHLKKKAVVTEKYEDDKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNDESFYGDFVLAYDILLKVDHIYDAINYVTQKPYSKDKFKLYFQPQFMGGWDKDKETDYATILYGSKYYLAIMPKKYAKCLQKIDKDDVNGNYEKINYKLLPGPNKMLPKVFFSKKWMAYYNPSEDIQKIYKNGTFKKGDMFNLNDCHKLIDFFKDSISYPKWSNAYDFNFSETEKYKDIAGFYEVEEQGYKVSFESASKKEVDKLVEEGKLYMFQIYNKDFSDKSHGTPNLHTMYFKLLFDENNHGQILSGGAELFMASLKKEELVVHPANSPIANKNPDNPKKTTTLSYDVYKDKFSEDQYELHIPIAINKCPKNIFKINTEVVLLKHDDNPYVIGIDGENLLYIVVVDGKGNIVEQYSLNEIINNFNGIIKTDYHSLLDKKEKEFEAQNWTSIENIKELKAGYISQVVHKICELVEKYDAVIALEDLNSGFKNSVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDIMYVPEEDLFEFALDYKNFSTDADYIKKWKLYSYGNIIFNPKKNNVFDWEEVCLTSAYKELFNKYGINYQQGDIALLCEQSDKAFYSSFMALMSLMLQMNSITGTDVDFLISPVKNSDGIFYDSNYEAQENAILPKNADANGAYNIAKVLWAIGQFKKAEDEKLDKVKIAISNKEWLEYAQTSVKH

SEQ ID NO: 7 - protein, 1185 aa, synthetic construct
MSKLEKFTNCYSLSKTLFKAIPVGKTQENIDNKLLVEDEKAEDYKGVKKLLDYYLSFINDVLHSIKLKNLNNYISLFKKTTEKENKELENLEINLKEIAKAFKGNEGYKSLFKKDIIETILPEFLDDKDEIALVNSFNGFTTAFTGFFDNENMFSEEAKSTSIAFCINENLTYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDESLSFYGEGYTSDEEVLEVFNTLNKNSEIFSSIKKLEKLFKNFDEYSSAGIFVKNGPAISTISKDIFGEWNVIDKWNAEYDDIHLKKKAVVTEKYEDDKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNDESFYGDFVLAYDILLKVDHIYDAINYVTQKPYSKDKFKLYFQPQFMGGWDKDKETDYATILYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISYPKWSNAYDFNFSETEKYKDIAGFYEVEEQGYKVSFESASKKEVDKLVEEGKLYMFQIYNKDFSDKSHGTPNLHTMYFKLLFDENNHGQILSGGAELFMASLKKEELVVHPANSPIANKNPDNPKKTTTLSYDVYKDKFSEDQYLLHIPIAINKCPKNIFKINTEVVLLKHDDNPYVIGIDGENLLYIVVVDGKGNIVEQYSLNEIINNFNGIIKTDYHSLLDKKEKEFEAQNWTSIENIKELKAGYISQVVHKICELVEKYDAVIALEDLNSGFKNSVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDIMYVPEEDLFEFALDYKNFSTDADYIKKWKLYSYGNIIFNPKKNNVFDWEEVCLTSAYKELFNKYGINYQQGDIALLCEQSDKAFYSSFMALMSLMLQMNSITGTDVDFLISPVKNSDGIFYDSNYEAQENAILPKNADANGAYNIAKVLWAIGQFKKAEDEKLDKVKIAISNKEWLEYAQTSVKH

SEQ ID NO: 8 - protein, 1186 aa, synthetic construct
MSKLEKFTNCYSLSKTLFKAIPVGKTQENIDNKLLVEDEKAEDYKGVKKLLDYYLSFINDVLHSIKLKNLNNYISLFKKTTEKENKELENLEINLKEIAKAFKGNEGYKSLFKKDIIETILPEFLDDKDEIALVNSFNGFTTAFTGFFDNENMFSEEAKSTSIAFCINENLTYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDESLSFYGEGYTSDEEVLEVFNTLNKNSEIFSSIKKLEKLFKNFDEYSSAGIFVKNGPAISTISKDIFGEWNVIDKWNAEYDDIHLKKKAVVTEKYEDDKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNDESFYGDFVLAYDILLKVDHIYDAINYVTQKPYSKDKFKLYFQNPQFMGGWDKDKETDYATILYGSKYYLAIMPKKYAKCLQKIDKDDVNGNYEKINYKLLPGPNKMLPKVFFSKKWMAYYNPSEDIQKIYKNGTFKKGDMFNLNDCHKLIDFFKDSISYPKWSNAYDFNFSETEKYKDIAGFYEVEEQGYKVSFESASKKEVDKLVEEGKLYMFQIYNKDFSDKSHGTPNLHTMYFKLLFDENNHGQILSGGAELFMASLKKEELVVHPANSPIANKNPDNPKKTTTLSYDVYKDKFSEDQYLLHIPIAINKCPKNIFKINTEVVLLKHDDNPYVIGIDGENLLYIVVVDGKGNIVEQYSLNEIINNFNGIIKTDYHSLLDKKEKEFEAQNWTSIENIKELKAGYISQVVHKICELVEKYDAVIALEDLNSGFKNSVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDIMYVPEEDLFEFALDYKNFSTDADYIKKWKLYSYGNIIFNPKKNNVFDWEEVCLTSAYKELFNKYGINYQQGDIALLCEQSDKAFYSSFMALMSLMLQMNSITGTDVDFLISPVKNSDGIFYDSNYEAQENAILPKNADANGAYNIAKVLWAIGQFKKAEDEKLDKVKIAISNKEWLEYAQTSVKH

SEQ ID NO: 9 - protein, 1185 aa, synthetic construct
MSKLEKFTNCYSLSKTLFKAIPVGKTQENIDNKLLVEDEKAEDYKGVKKLLDYYLSFINDVLHSIKLKNLNNYISLFKKTTEKENKELENLEINLKEIAKAFKGNEGYKSLFKKDIIETILPEFLDDKDEIALVNSFNGFTTAFTGFFDNENMFSEEAKSTSIAFCINENLTYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDESLSFYGEGYTSDEEVLEVFNTLNKNSEIFSSIKKLEKLFKNFDEYSSAGIFVKNGPAISTISKDIFGEWNVIDKWNAEYDDIHLKKKAVVTEKYEDDKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNDESFYGDFVLAYDILLKVDHIYDAINYVTQKPYSKDKFKLYFQPQFMGGWDKDKETDYATILYGSKYYLAIMPKKYAKCLQKIDKDDVNGNYEKINYKLLPGPNKMLPKVFFSKKWMAYYNPSEDIQKIYKNGTFKKGDMFNLNDCHKLIDFFKDSISYPKWSNAYDFNFSETEKYKDIAGFYEVEEQGYKVSFESASKKEVDKLVEEGKLYMFQIYNKDFSDKSHGTPNLHTMYFKLLFDENNHGQILSGGAELFMASLKKEELVVHPANSPIANKNPDNPKKTTTLSYDVYKDKFSEDQYLLHIPIAINKCPKNIFKINTEVVLLKHDDNPYVIGIDGENLLYIVVVDGKGNIVEQYSLNEIINNFNGIIKTDYHSLLDKKEKEFEAQNWTSIENIKELKAGYISQVVHKICELVEKYDAVIALEDLNSGFKNSVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDIMYVPEEDLFEFALDYKNFSTDADYIKKWKLYSYGNIIFNPKKNNVFDWEEVCLTSAYKELFNKYGINYQQGDIALLCEQSDKAFYSSFMALMSLMLQMNSITGTDVDFLISPVKNSDGIFYDSNYEAQENAILPKNADANGAYNIAKVLWAIGQFKKAEDEKLDKVKIAISNKEWLEYAQTSVKH

SEQ ID NO: 10 - DNA, 3684 bp, synthetic construct
  source 1..3684: mol_type = other DNA
atgagcaaactggaaaagttcaccaactgttatagcctgagcaaaaccctgcgttttaaagcaattccggttggtaaaacccaagagaacattgataataaacgcctgctggtcgaagatgaaaaacgcgctgaagattataaaggcgtgaaaaaactgctggatcgctattatctgagcttcattaacgatgtgctgcacagcattaaactgaagaacctgaacaactatatcagcctgtttcgtaaaaaaacccgcaccgaaaaagaaaacaaagagctggaaaacctggaaatcaatctgcgtaaagaaatcgccaaagcgtttaaaggtaacgagggttataaaagcctgttcaagaaagacatcatcgaaaccattctgccggaatttctggatgataaagatgaaattgccctggtgaatagctttaatggctttaccaccgcatttaccggcttttttgataatcgcgaaaacatgttcagcgaagaagcaaaaagcaccagcattgcatttcgctgcattaatgaaaatctgacccgctacattagcaacatggatatctttgaaaaagtggacgcgatcttcgataaacacgaagtgcaagagatcaaagagaaaatcctgaacagcgattatgacgtcgaagatttttttgaaggcgagttctttaacttcgttctgacccaagaaggtatcgacgtttataacgcaattattggtggttttgttaccgaaagcggtgagaaaatcaaaggcctgaatgaatatatcaacctgtataaccagaaaaccaaacagaaactgccgaaattcaaaccgctgtataaacaggttctgagcgatcgtgaaagcctgagcttttatggtgaaggttataccagtgatgaagaggttctggaagtttttcgtaacaccctgaataaaaacagcgagatctttagcagcatcaaaaagcttgagaaactgttcaaaaactttgatgagtatagcagcgcaggcatctttgttaaaaatggtccggcaattagcaccatcagcaaagatatttttggcgaatggaatgtgatccgcgataaatggaatgccgaatatgatgatatccacctgaaaaaaaaggccgtggtgaccgagaaatatgaagatgatcgtcgtaaaagcttcaagaaaattggtagctttagcctggaacagctgcaagaatatgcagatgcagatctgagcgttgtggaaaaactgaaagaaatcatcattcagaaggtggacgagatctataaagtttatggtagcagcgaaaaactgttcgatgcagattttgttctggaaaaaagcctgaaaaagaatgatgccgttgtggccattatgaaagatctgctggatagcgttaagagcttcgagaattacatcaaagccttttttggtgagggcaaagaaaccaatcgtgatgaaagtttctatggcgattttgtgctggcctatgatattctgctgaaagtggaccatatttatgatgccattcgcaattatgttacccagaaaccgtatagcaaagacaagttcaaactgtactttcagcgtccgcagtttatgggtggttgggataaagataaagaaaccgattatcgtgccaccatcctgcgttatggtagtaaatactatctggccatcatggacaaaaaatacgcaaaatgcctgcagaaaatcgacaaagatgatgtgaatggcaactatgaaaaaatcaactacaaactgctgcctggtccgaataaaatgctgccgaaagtgttctttagcaagaaatggatggcctattataacccgagcgaggatattcaaaagatctacaaaaatggcacctttaaaaagggcgacatgttcaatctgaacgattgccacaaactgatcgatttcttcaaagattcaatttcgcgttatccgaaatggtccaatgcctatgattttaactttagcgaaaccgaaaaatacaaagacattgccggtttttatcgcgaagtggaagaacagggctataaagtgagctttgaaagcgcaagcaaaaaagaggttgataagctggttgaagagggcaaactgtatatgttccagatttacaacaaagattttagcgacaaaagccatggcaccccgaatctgcataccatgtactttaaactgctgttcgacgaaaataaccatggtcagattcgtctgagcggtggtgccgaactgtttatgcgtcgtgcaagtctgaaaaaagaagaactggttgttcatccggcaaatagcccgattgcaaacaaaaatccggacaatccgaaaaaaaccacgacactgagctatgatgtgtataaagacaaacgttttagcgaggatcagtatgaactgcatatcccgattgccatcaataaatgcccgaaaaacatctttaagatcaacaccgaagttcgcgtgctgctgaaacatgatgataatccgtatgtgattggcattgatcgtggtgaacgtaacctgctgtatattgttgttgttgatggtaaaggcaacatcgtggaacagtatagtctgaacgaaattatcaacaactttaacggcatccgcatcaaaaccgactatcatagcctgctggacaagaaagaaaaagaacgttttgaagcacgtcagaactggaccagtattgaaaacatcaaagaactgaaagccggttatattagccaggtggttcataaaatctgtgagctggtagaaaaatacgatgcagttattgcactggaagatctgaatagcggtttcaaaaatagccgtgtgaaagtcgaaaaacaggtgtatcagaaattcgagaaaatgctgatcgacaaactgaactacatggtcgacaaaaaaagcaatccgtgtgcaaccggtggtgcactgaaaggttatcagattaccaacaaatttgaaagctttaaaagcatgagcacccagaacggctttatcttctatattccggcatggctgaccagcaaaattgatccgagcaccggttttgtgaacctgctgaaaacaaaatatacctccattgccgacagcaagaagtttattagcagctttgatcgcattatgtatgttccggaagaggacctgtttgaattcgcactggattacaaaaatttcagccgtaccgatgccgactacatcaaaaaatggaaactgtacagctatggtaaccgcattcgcatttttcgcaacccgaagaaaaacaatgtgttcgattgggaagaagtttgtctgaccagcgcatataaagaacttttcaacaaatacggcatcaactatcagcagggtgatattcgtgcactgctgtgtgaacagagcgataaagcgttttatagcagttttatggcactgatgagcctgatgctgcagatgcgtaatagcattaccggtcgcaccgatgtggattttctgattagtccggtgaaaaattccgatggcatcttttatgatagccgcaattacgaagcacaagaaaatgcaattctgccgaaaaacgcagatgcaaatggtgcatataacattgcacgtaaagttctgtgggcaattggccagtttaagaaagcagaagatgagaagctggacaaagtgaaaattgcgatcagcaataaagagtggctggaatacgcacagaccagcgttaaacat

SEQ ID NO: 11 - DNA, 3684 bp, synthetic construct
  source 1..3684: mol_type = other DNA
atgagcaaactggaaaagttcaccaactgttatagcctgagcaaaaccctgcgttttaaagcaattccggttggtaaaacccaagagaacattgataataaacgcctgctggtcgaagatgaaaaacgcgctgaagattataaaggcgtgaaaaaactgctggatcgctattatctgagcttcattaacgatgtgctgcacagcattaaactgaagaacctgaacaactatatcagcctgtttcgtaaaaaaacccgcaccgaaaaagaaaacaaagagctggaaaacctggaaatcaatctgcgtaaagaaatcgccaaagcgtttaaaggtaacgagggttataaaagcctgttcaagaaagacatcatcgaaaccattctgccggaatttctggatgataaagatgaaattgccctggtgaatagctttaatggctttaccaccgcatttaccggcttttttgataatcgcgaaaacatgttcagcgaagaagcaaaaagcaccagcattgcatttcgctgcattaatgaaaatctgacccgctacattagcaacatggatatctttgaaaaagtggacgcgatcttcgataaacacgaagtgcaagagatcaaagagaaaatcctgaacagcgattatgacgtcgaagatttttttgaaggcgagttctttaacttcgttctgacccaagaaggtatcgacgtttataacgcaattattggtggttttgttaccgaaagcggtgagaaaatcaaaggcctgaatgaatatatcaacctgtataaccagaaaaccaaacagaaactgccgaaattcaaaccgctgtataaacaggttctgagcgatcgtgaaagcctgagcttttatggtgaaggttataccagtgatgaagaggttctggaagtttttcgtaacaccctgaataaaaacagcgagatctttagcagcatcaaaaagcttgagaaactgttcaaaaactttgatgagtatagcagcgcaggcatctttgttaaaaatggtccggcaattagcaccatcagcaaagatatttttggcgaatggaatgtgatccgcgataaatggaatgccgaatatgatgatatccacctgaaaaaaaaggccgtggtgaccgagaaatatgaagatgatcgtcgtaaaagcttcaagaaaattggtagctttagcctggaacagctgcaagaatatgcagatgcagatctgagcgttgtggaaaaactgaaagaaatcatcattcagaaggtggacgagatctataaagtttatggtagcagcgaaaaactgttcgatgcagattttgttctggaaaaaagcctgaaaaagaatgatgccgttgtggccattatgaaagatctgctggatagcgttaagagcttcgagaattacatcaaagccttttttggtgagggcaaagaaaccaatcgtgatgaaagtttctatggcgattttgtgctggcctatgatattctgctgaaagtggaccatatttatgatgccattcgcaattatgttacccagaaaccgtatagcaaagacaagttcaaactgtactttcagaacccgcagtttatgggtggttgggataaagataaagaaaccgattatcgtgccaccatcctgcgttatggtagtaaatactatctggccatcatgccgaaaaaatacgcaaaatgcctgcagaaaatcgacaaagatgatgtgaatggcaactatgaaaaaatcaactacaaactgctgcctggtccgaataaaatgctgccgaaagtgttctttagcaagaaatggatggcctattataacccgagcgaggatattcaaaagatctacaaaaatggcacctttaaaaagggcgacatgttcaatctgaacgattgccacaaactgatcgatttcttcaaagattcaatttcgcgttatccgaaatggtccaatgcctatgattttaactttagcgaaaccgaaaaatacaaagacattgccggtttttatcgcgaagtggaagaacagggctataaagtgagctttgaaagcgcaagcaaaaaagaggttgataagctggttgaagagggcaaactgtatatgttccagatttacaacaaagattttagcgacaaaagccatggcaccccgaatctgcataccatgtactttaaactgctgttcgacgaaaataaccatggtcagattcgtctgagcggtggtgccgaactgtttatgcgtcgtgcaagtctgaaaaaagaagaactggttgttcatccggcaaatagcccgattgcaaacaaaaatccggacaatccgaaaaaaaccacgacactgagctatgatgtgtataaagacaaacgttttagcgaggatcagtatgaactgcatatcccgattgccatcaataaatgcccgaaaaacatctttaagatcaacaccgaagttcgcgtgctgctgaaacatgatgataatccgtatgtgattggcattgatcgtggtgaacgtaacctgctgtatattgttgttgttgatggtaaaggcaacatcgtggaacagtatagtctgaacgaaattatcaacaactttaacggcatccgcatcaaaaccgactatcatagcctgctggacaagaaagaaaaagaacgttttgaagcacgtcagaactggaccagtattgaaaacatcaaagaactgaaagccggttatattagccaggtggttcataaaatctgtgagctggtagaaaaatacgatgcagttattgcactggaagatctgaatagcggtttcaaaaatagccgtgtgaaagtcgaaaaacaggtgtatcagaaattcgagaaaatgctgatcgacaaactgaactacatggtcgacaaaaaaagcaatccgtgtgcaaccggtggtgcactgaaaggttatcagattaccaacaaatttgaaagctttaaaagcatgagcacccagaacggctttatcttctatattccggcatggctgaccagcaaaattgatccgagcaccggttttgtgaacctgctgaaaacaaaatatacctccattgccgacagcaagaagtttattagcagctttgatcgcattatgtatgttccggaagaggacctgtttgaattcgcactggattacaaaaatttcagccgtaccgatgccgactacatcaaaaaatggaaactgtacagctatggtaaccgcattcgcatttttcgcaacccgaagaaaaacaatgtgttcgattgggaagaagtttgtctgaccagcgcatataaagaacttttcaacaaatacggcatcaactatcagcagggtgatattcgtgcactgctgtgtgaacagagcgataaagcgttttatagcagttttatggcactgatgagcctgatgctgcagatgcgtaatagcattaccggtcgcaccgatgtggattttctgattagtccggtgaaaaattccgatggcatcttttatgatagccgcaattacgaagcacaagaaaatgcaattctgccgaaaaacgcagatgcaaatggtgcatataacattgcacgtaaagttctgtgggcaattggccagtttaagaaagcagaagatgagaagctggacaaagtgaaaattgcgatcagcaataaagagtggctggaatacgcacagaccagcgttaaacat

SEQ ID NO: 12 - DNA, 3684 bp, synthetic construct
  source 1..3684: mol_type = other DNA
atgagcaaactggaaaagttcaccaactgttatagcctgagcaaaaccctgcgttttaaagcaattccggttggtaaaacccaagagaacattgataataaacgcctgctggtcgaagatgaaaaacgcgctgaagattataaaggcgtgaaaaaactgctggatcgctattatctgagcttcattaacgatgtgctgcacagcattaaactgaagaacctgaacaactatatcagcctgtttcgtaaaaaaacccgcaccgaaaaagaaaacaaagagctggaaaacctggaaatcaatctgcgtaaagaaatcgccaaagcgtttaaaggtaacgagggttataaaagcctgttcaagaaagacatcatcgaaaccattctgccggaatttctggatgataaagatgaaattgccctggtgaatagctttaatggctttaccaccgcatttaccggcttttttgataatcgcgaaaacatgttcagcgaagaagcaaaaagcaccagcattgcatttcgctgcattaatgaaaatctgacccgctacattagcaacatggatatctttgaaaaagtggacgcgatcttcgataaacacgaagtgcaagagatcaaagagaaaatcctgaacagcgattatgacgtcgaagatttttttgaaggcgagttctttaacttcgttctgacccaagaaggtatcgacgtttataacgcaattattggtggttttgttaccgaaagcggtgagaaaatcaaaggcctgaatgaatatatcaacctgtataaccagaaaaccaaacagaaactgccgaaattcaaaccgctgtataaacaggttctgagcgatcgtgaaagcctgagcttttatggtgaaggttataccagtgatgaagaggttctggaagtttttcgtaacaccctgaataaaaacagcgagatctttagcagcatcaaaaagcttgagaaactgttcaaaaactttgatgagtatagcagcgcaggcatctttgttaaaaatggtccggcaattagcaccatcagcaaagatatttttggcgaatggaatgtgatccgcgataaatggaatgccgaatatgatgatatccacctgaaaaaaaaggccgtggtgaccgagaaatatgaagatgatcgtcgtaaaagcttcaagaaaattggtagctttagcctggaacagctgcaagaatatgcagatgcagatctgagcgttgtggaaaaactgaaagaaatcatcattcagaaggtggacgagatctataaagtttatggtagcagcgaaaaactgttcgatgcagattttgttctggaaaaaagcctgaaaaagaatgatgccgttgtggccattatgaaagatctgctggatagcgttaagagcttcgagaattacatcaaagccttttttggtgagggcaaagaaaccaatcgtgatgaaagtttctatggcgattttgtgctggcctatgatattctgctgaaagtggaccatatttatgatgccattcgcaattatgttacccagaaaccgtatagcaaagacaagttcaaactgtactttcagaacccgcagtttatgggtggttgggataaagataaagaaaccgattatcgtgccaccatcctgcgttatggtagtaaatactatctggccatcatggacaaaaaatacgcaaaatgcctgcagaaaatcgacaaagatgatgtgaatggcaactatgaaaaaatcaactacaaactgctgcctggtccgaataaaatgctgccgaaagtgttctttagcaagaaatggatggcctattataacccgagcgaggatattcaaaagatctacaaaaatggcacctttaaaaagggcgacatgttcaatctgaacgattgccacaaactgatcgatttcttcaaagattcaatttcgcgttatccgaaatggtccaatgcctatgattttaactttagcgaaaccgaaaaatacaaagacattgccggtttttatcgcgaagtggaagaacagggctataaagtgagctttgaaagcgcaagcaaaaaagaggttgataagctggttgaagagggcaaactgtatatgttccagatttacaacaaagattttagcgacaaaagccatggcaccccgaatctgcataccatgtactttaaactgctgttcgacgaaaataaccatggtcagattcgtctgagcggtggtgccgaactgtttatgcgtcgtgcaagtctgaaaaaagaagaactggttgttcatccggcaaatagcccgattgcaaacaaaaatccggacaatccgaaaaaaaccacgacactgagctatgatgtgtataaagacaaacgttttagcgaggatcagtatctgctgcatatcccgattgccatcaataaatgcccgaaaaacatctttaagatcaacaccgaagttcgcgtgctgctgaaacatgatgataatccgtatgtgattggcattgatcgtggtgaacgtaacctgctgtatattgttgttgttgatggtaaaggcaacatcgtggaacagtatagtctgaacgaaattatcaacaactttaacggcatccgcatcaaaaccgactatcatagcctgctggacaagaaagaaaaagaacgttttgaagcacgtcagaactggaccagtattgaaaacatcaaagaactgaaagccggttatattagccaggtggttcataaaatctgtgagctggtagaaaaatacgatgcagttattgcactggaagatctgaatagcggtttcaaaaatagccgtgtgaaagtcgaaaaacaggtgtatcagaaattcgagaaaatgctgatcgacaaactgaactacatggtcgacaaaaaaagcaatccgtgtgcaaccggtggtgcactgaaaggttatcagattaccaacaaatttgaaagctttaaaagcatgagcacccagaacggctttatcttctatattccggcatggctgaccagcaaaattgatccgagcaccggttttgtgaacctgctgaaaacaaaatatacctccattgccgacagcaagaagtttattagcagctttgatcgcattatgtatgttccggaagaggacctgtttgaattcgcactggattacaaaaatttcagccgtaccgatgccgactacatcaaaaaatggaaactgtacagctatggtaaccgcattcgcatttttcgcaacccgaagaaaaacaatgtgttcgattgggaagaagtttgtctgaccagcgcatataaagaacttttcaacaaatacggcatcaactatcagcagggtgatattcgtgcactgctgtgtgaacagagcgataaagcgttttatagcagttttatggcactgatgagcctgatgctgcagatgcgtaatagcattaccggtcgcaccgatgtggattttctgattagtccggtgaaaaattccgatggcatcttttatgatagccgcaattacgaagcacaagaaaatgcaattctgccgaaaaacgcagatgcaaatggtgcatataacattgcacgtaaagttctgtgggcaattggccagtttaagaaagcagaagatgagaagctggacaaagtgaaaattgcgatcagcaataaagagtggctggaatacgcacagaccagcgttaaacat

SEQ ID NO: 13 - DNA, 3684 bp, synthetic construct
  source 1..3684: mol_type = other DNA
atgagcaaactggaaaagttcaccaactgttatagcctgagcaaaaccctgcgttttaaagcaattccggttggtaaaacccaagagaacattgataataaacgcctgctggtcgaagatgaaaaacgcgctgaagattataaaggcgtgaaaaaactgctggatcgctattatctgagcttcattaacgatgtgctgcacagcattaaactgaagaacctgaacaactatatcagcctgtttcgtaaaaaaacccgcaccgaaaaagaaaacaaagagctggaaaacctggaaatcaatctgcgtaaagaaatcgccaaagcgtttaaaggtaacgagggttataaaagcctgttcaagaaagacatcatcgaaaccattctgccggaatttctggatgataaagatgaaattgccctggtgaatagctttaatggctttaccaccgcatttaccggcttttttgataatcgcgaaaacatgttcagcgaagaagcaaaaagcaccagcattgcatttcgctgcattaatgaaaatctgacccgctacattagcaacatggatatctttgaaaaagtggacgcgatcttcgataaacacgaagtgcaagagatcaaagagaaaatcctgaacagcgattatgacgtcgaagatttttttgaaggcgagttctttaacttcgttctgacccaagaaggtatcgacgtttataacgcaattattggtggttttgttaccgaaagcggtgagaaaatcaaaggcctgaatgaatatatcaacctgtataaccagaaaaccaaacagaaactgccgaaattcaaaccgctgtataaacaggttctgagcgatcgtgaaagcctgagcttttatggtgaaggttataccagtgatgaagaggttctggaagtttttcgtaacaccctgaataaaaacagcgagatctttagcagcatcaaaaagcttgagaaactgttcaaaaactttgatgagtatagcagcgcaggcatctttgttaaaaatggtccggcaattagcaccatcagcaaagatatttttggcgaatggaatgtgatccgcgataaatggaatgccgaatatgatgatatccacctgaaaaaaaaggccgtggtgaccgagaaatatgaagatgatcgtcgtaaaagcttcaagaaaattggtagctttagcctggaacagctgcaagaatatgcagatgcagatctgagcgttgtggaaaaactgaaagaaatcatcattcagaaggtggacgagatctataaagtttatggtagcagcgaaaaactgttcgatgcagattttgttctggaaaaaagcctgaaaaagaatgatgccgttgtggccattatgaaagatctgctggatagcgttaagagcttcgagaattacatcaaagccttttttggtgagggcaaagaaaccaatcgtgatgaaagtttctatggcgattttgtgctggcctatgatattctgctgaaagtggaccatatttatgatgccattcgcaattatgttacccagaaaccgtatagcaaagacaagttcaaactgtactttcagcgtccgcagtttatgggtggttgggataaagataaagaaaccgattatcgtgccaccatcctgcgttatggtagtaaatactatctggccatcatgccgaaaaaatacgcaaaatgcctgcagaaaatcgacaaagatgatgtgaatggcaactatgaaaaaatcaactacaaactgctgcctggtccgaataaaatgctgccgaaagtgttctttagcaagaaatggatggcctattataacccgagcgaggatattcaaaagatctacaaaaatggcacctttaaaaagggcgacatgttcaatctgaacgattgccacaaactgatcgatttcttcaaagattcaatttcgcgttatccgaaatggtccaatgcctatgattttaactttagcgaaaccgaaaaatacaaagacattgccggtttttatcgcgaagtggaagaacagggctataaagtgagctttgaaagcgcaagcaaaaaagaggttgataagctggttgaagagggcaaactgtatatgttccagatttacaacaaagattttagcgacaaaagccatggcaccccgaatctgcataccatgtactttaaactgctgttcgacgaaaataaccatggtcagattcgtctgagcggtggtgccgaactgtttatgcgtcgtgcaagtctgaaaaaagaagaactggttgttcatccggcaaatagcccgattgcaaacaaaaatccggacaatccgaaaaaaaccacgacactgagctatgatgtgtataaagacaaacgttttagcgaggatcagtatgaactgcatatcccgattgccatcaataaatgcccgaaaaacatctttaagatcaacaccgaagttcgcgtgctgctgaaacatgatgataatccgtatgtgattggcattgatcgtggtgaacgtaacctgctgtatattgttgttgttgatggtaaaggcaacatcgtggaacagtatagtctgaacgaaattatcaacaactttaacggcatccgcatcaaaaccgactatcatagcctgctggacaagaaagaaaaagaacgttttgaagcacgtcagaactggaccagtattgaaaacatcaaagaactgaaagccggttatattagccaggtggttcataaaatctgtgagctggtagaaaaatacgatgcagttattgcactggaagatctgaatagcggtttcaaaaatagccgtgtgaaagtcgaaaaacaggtgtatcagaaattcgagaaaatgctgatcgacaaactgaactacatggtcgacaaaaaaagcaatccgtgtgcaaccggtggtgcactgaaaggttatcagattaccaacaaatttgaaagctttaaaagcatgagcacccagaacggctttatcttctatattccggcatggctgaccagcaaaattgatccgagcaccggttttgtgaacctgctgaaaacaaaatatacctccattgccgacagcaagaagtttattagcagctttgatcgcattatgtatgttccggaagaggacctgtttgaattcgcactggattacaaaaatttcagccgtaccgatgccgactacatcaaaaaatggaaactgtacagctatggtaaccgcattcgcatttttcgcaacccgaagaaaaacaatgtgttcgattgggaagaagtttgtctgaccagcgcatataaagaacttttcaacaaatacggcatcaactatcagcagggtgatattcgtgcactgctgtgtgaacagagcgataaagcgttttatagcagttttatggcactgatgagcctgatgctgcagatgcgtaatagcattaccggtcgcaccgatgtggattttctgattagtccggtgaaaaattccgatggcatcttttatgatagccgcaattacgaagcacaagaaaatgcaattctgccgaaaaacgcagatgcaaatggtgcatataacattgcacgtaaagttctgtgggcaattggccagtttaagaaagcagaagatgagaagctggacaaagtgaaaattgcgatcagcaataaagagtggctggaatacgcacagaccagcgttaaacat

SEQ ID NO: 14 - DNA, 3684 bp, synthetic construct
  source 1..3684: mol_type = other DNA
atgagcaaactggaaaagttcaccaactgttatagcctgagcaaaaccctgcgttttaaagcaattccggttggtaaaacccaagagaacattgataataaacgcctgctggtcgaagatgaaaaacgcgctgaagattataaaggcgtgaaaaaactgctggatcgctattatctgagcttcattaacgatgtgctgcacagcattaaactgaagaacctgaacaactatatcagcctgtttcgtaaaaaaacccgcaccgaaaaagaaaacaaagagctggaaaacctggaaatcaatctgcgtaaagaaatcgccaaagcgtttaaaggtaacgagggttataaaagcctgttcaagaaagacatcatcgaaaccattctgccggaatttctggatgataaagatgaaattgccctggtgaatagctttaatggctttaccaccgcatttaccggcttttttgataatcgcgaaaacatgttcagcgaagaagcaaaaagcaccagcattgcatttcgctgcattaatgaaaatctgacccgctacattagcaacatggatatctttgaaaaagtggacgcgatcttcgataaacacgaagtgcaagagatcaaagagaaaatcctgaacagcgattatgacgtcgaagatttttttgaaggcgagttctttaacttcgttctgacccaagaaggtatcgacgtttataacgcaattattggtggttttgttaccgaaagcggtgagaaaatcaaaggcctgaatgaatatatcaacctgtataaccagaaaaccaaacagaaactgccgaaattcaaaccgctgtataaacaggttctgagcgatcgtgaaagcctgagcttttatggtgaaggttataccagtgatgaagaggttctggaagtttttcgtaacaccctgaataaaaacagcgagatctttagcagcatcaaaaagcttgagaaactgttcaaaaactttgatgagtatagcagcgcaggcatctttgttaaaaatggtccggcaattagcaccatcagcaaagatatttttggcgaatggaatgtgatccgcgataaatggaatgccgaatatgatgatatccacctgaaaaaaaaggccgtggtgaccgagaaatatgaagatgatcgtcgtaaaagcttcaagaaaattggtagctttagcctggaacagctgcaagaatatgcagatgcagatctgagcgttgtggaaaaactgaaagaaatcatcattcagaaggtggacgagatctataaagtttatggtagcagcgaaaaactgttcgatgcagattttgttctggaaaaaagcctgaaaaagaatgatgccgttgtggccattatgaaagatctgctggatagcgttaagagcttcgagaattacatcaaagccttttttggtgagggcaaagaaaccaatcgtgatgaaagtttctatggcgattttgtgctggcctatgatattctgctgaaagtggaccatatttatgatgccattcgcaattatgttacccagaaaccgtatagcaaagacaagttcaaactgtactttcagcgtccgcagtttatgggtggttgggataaagataaagaaaccgattatcgtgccaccatcctgcgttatggtagtaaatactatctggccatcatggacaaaaaatacgcaaaatgcctgcagaaaatcgacaaagatgatgtgaatggcaactatgaaaaaatcaactacaaactgctgcctggtccgaataaaatgctgccgaaagtgttctttagcaagaaatggatggcctattataacccgagcgaggatattcaaaagatctacaaaaatggcacctttaaaaagggcgacatgttcaatctgaacgattgccacaaactgatcgatttcttcaaagattcaatttcgcgttatccgaaatggtccaatgcctatgattttaactttagcgaaaccgaaaaatacaaagacattgccggtttttatcgcgaagtggaagaacagggctataaagtgagctttgaaagcgcaagcaaaaaagaggttgataagctggttgaagagggcaaactgtatatgttccagatttacaacaaagattttagcgacaaaagccatggcaccccgaatctgcataccatgtactttaaactgctgttcgacgaaaataaccatggtcagattcgtctgagcggtggtgccgaactgtttatgcgtcgtgcaagtctgaaaaaagaagaactggttgttcatccggcaaatagcccgattgcaaacaaaaatccggacaatccgaaaaaaaccacgacactgagctatgatgtgtataaagacaaacgttttagcgaggatcagtatctgctgcatatcccgattgccatcaataaatgcccgaaaaacatctttaagatcaacaccgaagttcgcgtgctgctgaaacatgatgataatccgtatgtgattggcattgatcgtggtgaacgtaacctgctgtatattgttgttgttgatggtaaaggcaacatcgtggaacagtatagtctgaacgaaattatcaacaactttaacggcatccgcatcaaaaccgactatcatagcctgctggacaagaaagaaaaagaacgttttgaagcacgtcagaactggaccagtattgaaaacatcaaagaactgaaagccggttatattagccaggtggttcataaaatctgtgagctggtagaaaaatacgatgcagttattgcactggaagatctgaatagcggtttcaaaaatagccgtgtgaaagtcgaaaaacaggtgtatcagaaattcgagaaaatgctgatcgacaaactgaactacatggtcgacaaaaaaagcaatccgtgtgcaaccggtggtgcactgaaaggttatcagattaccaacaaatttgaaagctttaaaagcatgagcacccagaacggctttatcttctatattccggcatggctgaccagcaaaattgatccgagcaccggttttgtgaacctgctgaaaacaaaatatacctccattgccgacagcaagaagtttattagcagctttgatcgcattatgtatgttccggaagaggacctgtttgaattcgcactggattacaaaaatttcagccgtaccgatgccgactacatcaaaaaatggaaactgtacagctatggtaaccgcattcgcatttttcgcaacccgaagaaaaacaatgtgttcgattgggaagaagtttgtctgaccagcgcatataaagaacttttcaacaaatacggcatcaactatcagcagggtgatattcgtgcactgctgtgtgaacagagcgataaagcgttttatagcagttttatggcactgatgagcctgatgctgcagatgcgtaatagcattaccggtcgcaccgatgtggattttctgattagtccggtgaaaaattccgatggcatcttttatgatagccgcaattacgaagcacaagaaaatgcaattctgccgaaaaacgcagatgcaaatggtgcatataacattgcacgtaaagttctgtgggcaattggccagtttaagaaagcagaagatgagaagctggacaaagtgaaaattgcgatcagcaataaagagtggctggaatacgcacagaccagcgttaaacat

SEQ ID NO: 15 - DNA, 3684 bp, synthetic construct
  source 1..3684: mol_type = other DNA
atgagcaaactggaaaagttcaccaactgttatagcctgagcaaaaccctgcgttttaaagcaattccggttggtaaaacccaagagaacattgataataaacgcctgctggtcgaagatgaaaaacgcgctgaagattataaaggcgtgaaaaaactgctggatcgctattatctgagcttcattaacgatgtgctgcacagcattaaactgaagaacctgaacaactatatcagcctgtttcgtaaaaaaacccgcaccgaaaaagaaaacaaagagctggaaaacctggaaatcaatctgcgtaaagaaatcgccaaagcgtttaaaggtaacgagggttataaaagcctgttcaagaaagacatcatcgaaaccattctgccggaatttctggatgataaagatgaaattgccctggtgaatagctttaatggctttaccaccgcatttaccggcttttttgataatcgcgaaaacatgttcagcgaagaagcaaaaagcaccagcattgcatttcgctgcattaatgaaaatctgacccgctacattagcaacatggatatctttgaaaaagtggacgcgatcttcgataaacacgaagtgcaagagatcaaagagaaaatcctgaacagcgattatgacgtcgaagatttttttgaaggcgagttctttaacttcgttctgacccaagaaggtatcgacgtttataacgcaattattggtggttttgttaccgaaagcggtgagaaaatcaaaggcctgaatgaatatatcaacctgtataaccagaaaaccaaacagaaactgccgaaattcaaaccgctgtataaacaggttctgagcgatcgtgaaagcctgagcttttatggtgaaggttataccagtgatgaagaggttctggaagtttttcgtaacaccctgaataaaaacagcgagatctttagcagcatcaaaaagcttgagaaactgttcaaaaactttgatgagtatagcagcgcaggcatctttgttaaaaatggtccggcaattagcaccatcagcaaagatatttttggcgaatggaatgtgatccgcgataaatggaatgccgaatatgatgatatccacctgaaaaaaaaggccgtggtgaccgagaaatatgaagatgatcgtcgtaaaagcttcaagaaaattggtagctttagcctggaacagctgcaagaatatgcagatgcagatctgagcgttgtggaaaaactgaaagaaatcatcattcagaaggtggacgagatctataaagtttatggtagcagcgaaaaactgttcgatgcagattttgttctggaaaaaagcctgaaaaagaatgatgccgttgtggccattatgaaagatctgctggatagcgttaagagcttcgagaattacatcaaagccttttttggtgagggcaaagaaaccaatcgtgatgaaagtttctatggcgattttgtgctggcctatgatattctgctgaaagtggaccatatttatgatgccattcgcaattatgttacccagaaaccgtatagcaaagacaagttcaaactgtactttcagaacccgcagtttatgggtggttgggataaagataaagaaaccgattatcgtgccaccatcctgcgttatggtagtaaatactatctggccatcatgccgaaaaaatacgcaaaatgcctgcagaaaatcgacaaagatgatgtgaatggcaactatgaaaaaatcaactacaaactgctgcctggtccgaataaaatgctgccgaaagtgttctttagcaagaaatggatggcctattataacccgagcgaggatattcaaaagatctacaaaaatggcacctttaaaaagggcgacatgttcaatctgaacgattgccacaaactgatcgatttcttcaaagattcaatttcgcgttatccgaaatggtccaatgcctatgattttaactttagcgaaaccgaaaaatacaaagacattgccggtttttatcgcgaagtggaagaacagggctataaagtgagctttgaaagcgcaagcaaaaaagaggttgataagctggttgaagagggcaaactgtatatgttccagatttacaacaaagattttagcgacaaaagccatggcaccccgaatctgcataccatgtactttaaactgctgttcgacgaaaataaccatggtcagattcgtctgagcggtggtgccgaactgtttatgcgtcgtgcaagtctgaaaaaagaagaactggttgttcatccggcaaatagcccgattgcaaacaaaaatccggacaatccgaaaaaaaccacgacactgagctatgatgtgtataaagacaaacgttttagcgaggatcagtatctgctgcatatcccgattgccatcaataaatgcccgaaaaacatctttaagatcaacaccgaagttcgcgtgctgctgaaacatgatgataatccgtatgtgattggcattgatcgtggtgaacgtaacctgctgtatattgttgttgttgatggtaaaggcaacatcgtggaacagtatagtctgaacgaaattatcaacaactttaacggcatccgcatcaaaaccgactatcatagcctgctggacaagaaagaaaaagaacgttttgaagcacgtcagaactggaccagtattgaaaacatcaaagaactgaaagccggttatattagccaggtggttcataaaatctgtgagctggtagaaaaatacgatgcagttattgcactggaagatctgaatagcggtttcaaaaatagccgtgtgaaagtcgaaaaacaggtgtatcagaaattcgagaaaatgctgatcgacaaactgaactacatggtcgacaaaaaaagcaatccgtgtgcaaccggtggtgcactgaaaggttatcagattaccaacaaatttgaaagctttaaaagcatgagcacccagaacggctttatcttctatattccggcatggctgaccagcaaaattgatccgagcaccggttttgtgaacctgctgaaaacaaaatatacctccattgccgacagcaagaagtttattagcagctttgatcgcattatgtatgttccggaagaggacctgtttgaattcgcactggattacaaaaatttcagccgtaccgatgccgactacatcaaaaaatggaaactgtacagctatggtaaccgcattcgcatttttcgcaacccgaagaaaaacaatgtgttcgattgggaagaagtttgtctgaccagcgcatataaagaacttttcaacaaatacggcatcaactatcagcagggtgatattcgtgcactgctgtgtgaacagagcgataaagcgttttatagcagttttatggcactgatgagcctgatgctgcagatgcgtaatagcattaccggtcgcaccgatgtggattttctgattagtccggtgaaaaattccgatggcatcttttatgatagccgcaattacgaagcacaagaaaatgcaattctgccgaaaaacgcagatgcaaatggtgcatataacattgcacgtaaagttctgtgggcaattggccagtttaagaaagcagaagatgagaagctggacaaagtgaaaattgcgatcagcaataaagagtggctggaatacgcacagaccagcgttaaacat

SEQ ID NO: 16 - DNA, 3684 bp, synthetic construct
  source 1..3684: mol_type = other DNA
atgagcaaactggaaaagttcaccaactgttatagcctgagcaaaaccctgcgttttaaagcaattccggttggtaaaacccaagagaacattgataataaacgcctgctggtcgaagatgaaaaacgcgctgaagattataaaggcgtgaaaaaactgctggatcgctattatctgagcttcattaacgatgtgctgcacagcattaaactgaagaacctgaacaactatatcagcctgtttcgtaaaaaaacccgcaccgaaaaagaaaacaaagagctggaaaacctggaaatcaatctgcgtaaagaaatcgccaaagcgtttaaaggtaacgagggttataaaagcctgttcaagaaagacatcatcgaaaccattctgccggaatttctggatgataaagatgaaattgccctggtgaatagctttaatggctttaccaccgcatttaccggcttttttgataatcgcgaaaacatgttcagcgaagaagcaaaaagcaccagcattgcatttcgctgcattaatgaaaatctgacccgctacattagcaacatggatatctttgaaaaagtggacgcgatcttcgataaacacgaagtgcaagagatcaaagagaaaatcctgaacagcgattatgacgtcgaagatttttttgaaggcgagttctttaacttcgttctgacccaagaaggtatcgacgtttataacgcaattattggtggttttgttaccgaaagcggtgagaaaatcaaaggcctgaatgaatatatcaacctgtataaccagaaaaccaaacagaaactgccgaaattcaaaccgctgtataaacaggttctgagcgatcgtgaaagcctgagcttttatggtgaaggttataccagtgatgaagaggttctggaagtttttcgtaacaccctgaataaaaacagcgagatctttagcagcatcaaaaagcttgagaaactgttcaaaaactttgatgagtatagcagcgcaggcatctttgttaaaaatggtccggcaattagcaccatcagcaaagatatttttggcgaatggaatgtgatccgcgataaatggaatgccgaatatgatgatatccacctgaaaaaaaaggccgtggtgaccgagaaatatgaagatgatcgtcgtaaaagcttcaagaaaattggtagctttagcctggaacagctgcaagaatatgcagatgcagatctgagcgttgtggaaaaactgaaagaaatcatcattcagaaggtggacgagatctataaagtttatggtagcagcgaaaaactgttcgatgcagattttgttctggaaaaaagcctgaaaaagaatgatgccgttgtggccattatgaaagatctgctggatagcgttaagagcttcgagaattacatcaaagccttttttggtgagggcaaagaaaccaatcgtgatgaaagtttctatggcgattttgtgctggcctatgatattctgctgaaagtggaccatatttatgatgccattcgcaattatgttacccagaaaccgtatagcaaagacaagttcaaactgtactttcagcgtccgcagtttatgggtggttgggataaagataaagaaaccgattatcgtgccaccatcctgcgttatggtagtaaatactatctggccatcatgccgaaaaaatacgcaaaatgcctgcagaaaatcgacaaagatgatgtgaatggcaactatgaaaaaatcaactacaaactgctgcctggtccgaataaaatgctgccgaaagtgttctttagcaagaaatggatggcctattataacccgagcgaggatattcaaaagatctacaaaaatggcacctttaaaaagggcgacatgttcaatctgaacgattgccacaaactgatcgatttcttcaaagattcaatttcgcgttatccgaaatggtccaatgcctatgattttaactttagcgaaaccgaaaaatacaaagacattgccggtttttatcgcgaagtggaagaacagggctataaagtgagctttgaaagcgcaagcaaaaaagaggttgataagctggttgaagagggcaaactgtatatgttccagatttacaacaaagattttagcgacaaaagccatggcaccccgaatctgcataccatgtactttaaactgctgttcgacgaaaataaccatggtcagattcgtctgagcggtggtgccgaactgtttatgcgtcgtgcaagtctgaaaaaagaagaactggttgttcatccggcaaatagcccgattgcaaacaaaaatccggacaatccgaaaaaaaccacgacactgagctatgatgtgtataaagacaaacgttttagcgaggatcagtatctgctgcatatcccgattgccatcaataaatgcccgaaaaacatctttaagatcaacaccgaagttcgcgtgctgctgaaacatgatgataatccgtatgtgattggcattgatcgtggtgaacgtaacctgctgtatattgttgttgttgatggtaaaggcaacatcgtggaacagtatagtctgaacgaaattatcaacaactttaacggcatccgcatcaaaaccgactatcatagcctgctggacaagaaagaaaaagaacgttttgaagcacgtcagaactggaccagtattgaaaacatcaaagaactgaaagccggttatattagccaggtggttcataaaatctgtgagctggtagaaaaatacgatgcagttattgcactggaagatctgaatagcggtttcaaaaatagccgtgtgaaagtcgaaaaacaggtgtatcagaaattcgagaaaatgctgatcgacaaactgaactacatggtcgacaaaaaaagcaatccgtgtgcaaccggtggtgcactgaaaggttatcagattaccaacaaatttgaaagctttaaaagcatgagcacccagaacggctttatcttctatattccggcatggctgaccagcaaaattgatccgagcaccggttttgtgaacctgctgaaaacaaaatatacctccattgccgacagcaagaagtttattagcagctttgatcgcattatgtatgttccggaagaggacctgtttgaattcgcactggattacaaaaatttcagccgtaccgatgccgactacatcaaaaaatggaaactgtacagctatggtaaccgcattcgcatttttcgcaacccgaagaaaaacaatgtgttcgattgggaagaagtttgtctgaccagcgcatataaagaacttttcaacaaatacggcatcaactatcagcagggtgatattcgtgcactgctgtgtgaacagagcgataaagcgttttatagcagttttatggcactgatgagcctgatgctgcagatgcgtaatagcattaccggtcgcaccgatgtggattttctgattagtccggtgaaaaattccgatggcatcttttatgatagccgcaattacgaagcacaagaaaatgcaattctgccgaaaaacgcagatgcaaatggtgcatataacattgcacgtaaagttctgtgggcaattggccagtttaagaaagcagaagatgagaagctggacaaagtgaaaattgcgatcagcaataaagagtggctggaatacgcacagaccagcgttaaacat

SEQ ID NO: 17 - DNA, 3921 bp, unidentified
  source 1..3921: mol_type = other DNA; note = Acidaminococcus sp.
atgacccagtttgaaggtttcaccaatctgtatcaggttagcaaaaccctgcgttttgaactgattccgcagggtaaaaccctgaaacatattcaagaacagggcttcatcgaagaggataaagcacgtaacgatcactacaaagaactgaaaccgattatcgaccgcatctataaaacctatgcagatcagtgtctgcagctggttcagctggattgggaaaatctgagcgcagcaattgatagttatcgcaaagaaaaaaccgaagaaacccgtaatgcactgattgaagaacaggcaacctatcgtaatgccatccatgattatttcattggtcgtaccgataatctgaccgatgcaattaacaaacgtcacgccgaaatctataaaggcctgtttaaagccgaactgtttaatggcaaagttctgaaacagctgggcaccgttaccaccaccgaacatgaaaatgcactgctgcgtagctttgataaattcaccacctatttcagcggcttttatgagaatcgcaaaaacgtgtttagcgcagaagatattagcaccgcaattccgcatcgtattgtgcaggataatttcccgaaattcaaagagaactgccacatttttacccgtctgattaccgcagttccgagcctgcgtgaacattttgaaaacgttaaaaaagccatcggcatctttgttagcaccagcattgaagaagtttttagcttcccgttttacaatcagctgctgacccagacccagattgatctgtataaccaactgctgggtggtattagccgtgaagcaggcaccgaaaaaatcaaaggtctgaatgaagtgctgaatctggccattcagaaaaatgatgaaaccgcacatattattgcaagcctgccgcatcgttttattccgctgttcaaacaaattctgagcgatcgtaataccctgagctttattctggaagaattcaaatccgatgaagaggtgattcagagcttttgcaaatacaaaacgctgctgcgcaatgaaaatgttctggaaactgccgaagcactgtttaacgaactgaatagcattgatctgacccacatctttatcagccacaaaaaactggaaaccatttcaagcgcactgtgtgatcattgggataccctgcgtaatgccctgtatgaacgtcgtattagcgaactgaccggtaaaattaccaaaagcgcgaaagaaaaagttcagcgcagtctgaaacatgaggatattaatctgcaagagattattagcgcagccggtaaagaactgtcagaagcatttaaacagaaaaccagcgaaattctgtcacatgcacatgcagcactggatcagccgctgccgaccaccctgaaaaaacaagaagaaaaagaaatcctgaaaagccagctggatagcctgctgggtctgtatcatctgctggactggtttgcagttgatgaaagcaatgaagttgatccggaatttagcgcacgtctgaccggcattaaactggaaatggaaccgagcctgagcttttataacaaagcccgtaattatgccaccaaaaaaccgtatagcgtcgaaaaattcaaactgaactttcagatgccgaccctggcaagcggttgggatgttaataaagaaaaaaacaacggtgccatcctgttcgtgaaaaatggcctgtattatctgggtattatgccgaaacagaaaggtcgttataaagcgctgagctttgaaccgacggaaaaaaccagtgaaggttttgataaaatgtactacgactattttccggatgcagccaaaatgattccgaaatgtagcacccagctgaaagcagttaccgcacattttcagacccataccaccccgattctgctgagcaataactttattgaaccgctggaaatcaccaaagagatctacgatctgaataacccggaaaaagagccgaaaaaattccagaccgcatatgcaaaaaaaaccggtgatcagaaaggttatcgtgaagcgctgtgtaaatggattgatttcacccgtgattttctgagcaaatacaccaaaaccaccagtatcgatctgagcagcctgcgtccgagcagccagtataaagatctgggcgaatattatgcagaactgaatccgctgctgtatcatattagctttcagcgtattgccgagaaagaaatcatggacgcagttgaaaccggtaaactgtacctgttccagatctacaataaagattttgccaaaggccatcatggcaaaccgaatctgcataccctgtattggaccggtctgtttagccctgaaaatctggcaaaaacctcgattaaactgaatggtcaggcggaactgttttatcgtccgaaaagccgtatgaaacgtatggcacatcgtctgggtgaaaaaatgctgaacaaaaaactgaaagaccagaaaaccccgatcccggatacactgtatcaagaactgtatgattatgtgaaccatcgtctgagccatgatctgagtgatgaagcacgtgccctgctgccgaatgttattaccaaagaagttagccacgagatcattaaagatcgtcgttttaccagcgacaaattcttttttcatgtgccgattaccctgaattatcaggcagcaaatagcccgagcaaatttaaccagcgtgttaatgcatatctgaaagaacatccagaaacgccgattattggtattgatcgtggtgaacgtaacctgatttatatcaccgttattgatagcaccggcaaaatcctggaacagcgtagcctgaataccattcagcagtttgattaccagaaaaaactggataatcgcgagaaagaacgtgttgcagcacgtcaggcatggtcagttgttggtacaattaaagacctgaaacagggttatctgagccaggttattcatgaaattgtggatctgatgattcactatcaggccgttgttgtgctggaaaacctgaattttggctttaaaagcaaacgtaccggcattgcagaaaaagcagtttatcagcagttcgagaaaatgctgattgacaaactgaattgcctggtgctgaaagattatccggctgaaaaagttggtggtgttctgaatccgtatcagctgaccgatcagtttaccagctttgcaaaaatgggcacccagagcggatttctgttttatgttccggcaccgtatacgagcaaaattgatccgctgaccggttttgttgatccgtttgtttggaaaaccatcaaaaaccatgaaagccgcaaacattttctggaaggtttcgattttctgcattacgacgttaaaacgggtgatttcatcctgcactttaaaatgaatcgcaatctgagttttcagcgtggcctgcctggttttatgcctgcatgggatattgtgtttgagaaaaacgaaacacagttcgatgcaaaaggcaccccgtttattgcaggtaaacgtattgttccggtgattgaaaatcatcgtttcaccggtcgttatcgcgatctgtatccggcaaatgaactgatcgcactgctggaagagaaaggtattgtttttcgtgatggctcaaacattctgccgaaactgctggaaaatgatgatagccatgcaattgataccatggttgcactgattcgtagcgttctgcagatgcgtaatagcaatgcagcaaccggtgaagattacattaatagtccggttcgtgatctgaatggtgtttgttttgatagccgttttcagaatccggaatggccgatggatgcagatgcaaatggtgcatatcatattgcactgaaaggacagctgctgctgaaccacctgaaagaaagcaaagatctgaaactgcaaaacggcattagcaatcaggattggctggcatatatccaagaactgcgtaac

SEQ ID NO: 18 - protein, 1250 aa, unidentified
  source 1..1250: note = Acidaminococcus sp.
MTQFEGFTNLYQVSKTLFELIPQGKTLKHIQEQGFIEEDKANDHYKELKPIIDIYKTYADQCLQLVQLDWENLSAAIDSYKEKTEETNALIEEQATYNAIHDYFIGTDNLTDAINKHAEIYKGLFKAELFNGKVLKQLGTVTTTEHENALLSFDKFTTYFSGFYENKNVFSAEDISTAIPHIVQDNFPKFKENCHIFTLITAVPSLEHFENVKKAIGIFVSTSIEEVFSFPFYNQLLTQTQIDLYNQLLGGISEAGTEKIKGLNEVLNLAIQKNDETAHIIASLPHFIPLFKQILSDNTLSFILEEFKSDEEVIQSFCKYKTLLNENVLETAEALFNELNSIDLTHIFISHKKLETISSALCDHWDTLNALYEISELTGKITKSAKEKVQSLKHEDINLQEIISAAGKELSEAFKQKTSEILSHAHAALDQPLPTTLKKQEEKEILKSQLDSLLGLYHLLDWFAVDESNEVDPEFSALTGIKLEMEPSLSFYNKANYATKKPYSVEKFKLNFQMPTLASGWDVNKEKNNGAILFVKNGLYYLGIMPKQKGYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYEALCKWIDFTDFLSKYTKTTSIDLSSLPSSQYKDLGEYYAELNPLLYHISFQIAEKEIMDAVETGKLYLFQIYNKDFAKGHHGKPNLHTLYWTGLFSPENLAKTSIKLNGQAELFYPKSMKMAHLGEKMLNKKLKDQKTPIPDTLYQELYDYVNHLSHDLSDEAALLPNVITKEVSHEIIKDFTSDKFFFHVPITLNYQAANSPSKFNQVNAYLKEHPETPIIGIDGENLIYITVIDSTGKILEQSLNTIQQFDYQKKLDNEKEVAAQAWSVVGTIKDLKQGYLSQVIHEIVDLMIHYQAVVVLENLNFGFKSKTGIAEKAVYQQFEKMLIDKLNCLVLKDYPAEKVGGVLNPYQLTDQFTSFAKMGTQSGFLFYVPAPYTSKIDPLTGFVDPFVWKTIKNHESKHFLEGFDFLHYDVKTGDFILHFKMNNLSFQGLPGFMPAWDIVFEKNETQFDAKGTPFIAGKIVPVIENHFTGYDLYPANELIALLEEKGIVFDGSNILPKLLENDDSHAIDTMVALISVLQMNSNAATGEDYINSPVDLNGVCFDSFQNPEWPMDADANGAYHIALKGQLLLNHLKESKDLKLQNGISNQDWLAYIQELN

SEQ ID NO: 19 - DNA, 3921 bp, synthetic construct
  source 1..3921: mol_type = other DNA
atgacccagtttgaaggtttcaccaatctgtatcaggttagcaaaaccctgcgttttgaactgattccgcagggtaaaaccctgaaacatattcaagaacagggcttcatcgaagaggataaagcacgtaacgatcactacaaagaactgaaaccgattatcgaccgcatctataaaacctatgcagatcagtgtctgcagctggttcagctggattgggaaaatctgagcgcagcaattgatagttatcgcaaagaaaaaaccgaagaaacccgtaatgcactgattgaagaacaggcaacctatcgtaatgccatccatgattatttcattggtcgtaccgataatctgaccgatgcaattaacaaacgtcacgccgaaatctataaaggcctgtttaaagccgaactgtttaatggcaaagttctgaaacagctgggcaccgttaccaccaccgaacatgaaaatgcactgctgcgtagctttgataaattcaccacctatttcagcggcttttatgagaatcgcaaaaacgtgtttagcgcagaagatattagcaccgcaattccgcatcgtattgtgcaggataatttcccgaaattcaaagagaactgccacatttttacccgtctgattaccgcagttccgagcctgcgtgaacattttgaaaacgttaaaaaagccatcggcatctttgttagcaccagcattgaagaagtttttagcttcccgttttacaatcagctgctgacccagacccagattgatctgtataaccaactgctgggtggtattagccgtgaagcaggcaccgaaaaaatcaaaggtctgaatgaagtgctgaatctggccattcagaaaaatgatgaaaccgcacatattattgcaagcctgccgcatcgttttattccgctgttcaaacaaattctgagcgatcgtaataccctgagctttattctggaagaattcaaatccgatgaagaggtgattcagagcttttgcaaatacaaaacgctgctgcgcaatgaaaatgttctggaaactgccgaagcactgtttaacgaactgaatagcattgatctgacccacatctttatcagccacaaaaaactggaaaccatttcaagcgcactgtgtgatcattgggataccctgcgtaatgccctgtatgaacgtcgtattagcgaactgaccggtaaaattaccaaaagcgcgaaagaaaaagttcagcgcagtctgaaacatgaggatattaatctgcaagagattattagcgcagccggtaaagaactgtcagaagcatttaaacagaaaaccagcgaaattctgtcacatgcacatgcagcactggatcagccgctgccgaccaccctgaaaaaacaagaagaaaaagaaatcctgaaaagccagctggatagcctgctgggtctgtatcatctgctggactggtttgcagttgatgaaagcaatgaagttgatccggaatttagcgcacgtctgaccggcattaaactggaaatggaaccgagcctgagcttttataacaaagcccgtaattatgccaccaaaaaaccgtatagcgtcgaaaaattcaaactgaactttcagcgtccgaccctggcaagcggttgggatgttaataaagaaaaaaacaacggtgccatcctgttcgtgaaaaatggcctgtattatctgggtattatgccgaaacagaaaggtcgttataaagcgctgagctttgaaccgacggaaaaaaccagtgaaggttttgataaaatgtactacgactattttccggatgcagccaaaatgattccgaaatgtagcacccagctgaaagcagttaccgcacattttcagacccataccaccccgattctgctgagcaataactttattgaaccgctggaaatcaccaaagagatctacgatctgaataacccggaaaaagagccgaaaaaattccagaccgcatatgcaaaaaaaaccggtgatcagaaaggttatcgtgaagcgctgtgtaaatggattgatttcacccgtgattttctgagcaaatacaccaaaaccaccagtatcgatctgagcagcctgcgtccgagcagccagtataaagatctgggcgaatattatgcagaactgaatccgctgctgtatcatattagctttcagcgtattgccgagaaagaaatcatggacgcagttgaaaccggtaaactgtacctgttccagatctacaataaagattttgccaaaggccatcatggcaaaccgaatctgcataccctgtattggaccggtctgtttagccctgaaaatctggcaaaaacctcgattaaactgaatggtcaggcggaactgttttatcgtccgaaaagccgtatgaaacgtatggcacatcgtctgggtgaaaaaatgctgaacaaaaaactgaaagaccagaaaaccccgatcccggatacactgtatcaagaactgtatgattatgtgaaccatcgtctgagccatgatctgagtgatgaagcacgtgccctgctgccgaatgttattaccaaagaagttagccacgagatcattaaagatcgtcgttttaccagcgacaaattcctgtttcatgtgccgattaccctgaattatcaggcagcaaatagcccgagcaaatttaaccagcgtgttaatgcatatctgaaagaacatccagaaacgccgattattggtattgatcgtggtgaacgtaacctgatttatatcaccgttattgatagcaccggcaaaatcctggaacagcgtagcctgaataccattcagcagtttgattaccagaaaaaactggataatcgcgagaaagaacgtgttgcagcacgtcaggcatggtcagttgttggtacaattaaagacctgaaacagggttatctgagccaggttattcatgaaattgtggatctgatgattcactatcaggccgttgttgtgctggaaaacctgaattttggctttaaaagcaaacgtaccggcattgcagaaaaagcagtttatcagcagttcgagaaaatgctgattgacaaactgaattgcctggtgctgaaagattatccggctgaaaaagttggtggtgttctgaatccgtatcagctgaccgatcagtttaccagctttgcaaaaatgggcacccagagcggatttctgttttatgttccggcaccgtatacgagcaaaattgatccgctgaccggttttgttgatccgtttgtttggaaaaccatcaaaaaccatgaaagccgcaaacattttctggaaggtttcgattttctgcattacgacgttaaaacgggtgatttcatcctgcactttaaaatgaatcgcaatctgagttttcagcgtggcctgcctggttttatgcctgcatgggatattgtgtttgagaaaaacgaaacacagttcgatgcaaaaggcaccccgtttattgcaggtaaacgtattgttccggtgattgaaaatcatcgtttcaccggtcgttatcgcgatctgtatccggcaaatgaactgatcgcactgctggaagagaaaggtattgtttttcgtgatggctcaaacattctgccgaaactgctggaaaatgatgatagccatgcaattgataccatggttgcactgattcgtagcgttctgcagatgcgtaatagcaatgcagcaaccggtgaagattacattaatagtccggttcgtgatctgaatggtgtttgttttgatagccgttttcagaatccggaatggccgatggatgcagatgcaaatggtgcatatcatattgcactgaaaggacagctgctgctgaaccacctgaaagaaagcaaagatctgaaactgcaaaacggcattagcaatcaggattggctggcatatatccaagaactgcgtaac

SEQ ID NO: 20 - protein, 1249 aa, synthetic construct
MTQFEGFTNLYQVSKTLFELIPQGKTLKHIQEQGFIEEDKANDHYKELKPIIDIYKTYADQCLQLVQLDWENLSAAIDSYKEKTEETNALIEEQATYNAIHDYFIGTDNLTDAINKHAEIYKGLFKAELFNGKVLKQLGTVTTTEHENALLSFDKFTTYFSGFYENKNVFSAEDISTAIPHIVQDNFPKFKENCHIFTLITAVPSLEHFENVKKAIGIFVSTSIEEVFSFPFYNQLLTQTQIDLYNQLLGGISEAGTEKIKGLNEVLNLAIQKNDETAHIIASLPHFIPLFKQILSDNTLSFILEEFKSDEEVIQSFCKYKTLLNENVLETAEALFNELNSIDLTHIFISHKKLETISSALCDHWDTLNALYEISELTGKITKSAKEKVQSLKHEDINLQEIISAAGKELSEAFKQKTSEILSHAHAALDQPLPTTLKKQEEKEILKSQLDSLLGLYHLLDWFAVDESNEVDPEFSALTGIKLEMEPSLSFYNKANYATKKPYSVEKFKLNFQPTLASGWDVNKEKNNGAILFVKNGLYYLGIMPKQKGYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYEALCKWIDFTDFLSKYTKTTSIDLSSLPSSQYKDLGEYYAELNPLLYHISFQIAEKEIMDAVETGKLYLFQIYNKDFAKGHHGKPNLHTLYWTGLFSPENLAKTSIKLNGQAELFYPKSMKMAHLGEKMLNKKLKDQKTPIPDTLYQELYDYVNHLSHDLSDEAALLPNVITKEVSHEIIKDFTSDKFLFHVPITLNYQAANSPSKFNQVNAYLKEHPETPIIGIDGENLIYITVIDSTGKILEQSLNTIQQFDYQKKLDNEKEVAAQAWSVVGTIKDLKQGYLSQVIHEIVDLMIHYQAVVVLENLNFGFKSKTGIAEKAVYQQFEKMLIDKLNCLVLKDYPAEKVGGVLNPYQLTDQFTSFAKMGTQSGFLFYVPAPYTSKIDPLTGFVDPFVWKTIKNHESKHFLEGFDFLHYDVKTGDFILHFKMNNLSFQGLPGFMPAWDIVFEKNETQFDAKGTPFIAGKIVPVIENHFTGYDLYPANELIALLEEKGIVFDGSNILPKLLENDDSHAIDTMVALISVLQMNSNAATGEDYINSPVDLNGVCFDSFQNPEWPMDADANGAYHIALKGQLLLNHLKESKDLKLQNGISNQDWLAYIQELN

SEQ ID NO: 21 - DNA, 53 bp, synthetic construct
  source 1..53: mol_type = other DNA
gcaaagacaagttcaaactgtactttcagcgtccgcagtttatgggtggttgg

SEQ ID NO: 22 - DNA, 53 bp, synthetic construct
  source 1..53: mol_type = other DNA
ccaaccacccataaactgcggacgctgaaagtacagtttgaacttgtctttgc

SEQ ID NO: 23 - DNA, 58 bp, synthetic construct
  source 1..58: mol_type = other DNA
tatggtagtaaatactatctggccatcatgccgaaaaaatacgcaaaatgcctgcaga

SEQ ID NO: 24 - DNA, 58 bp, synthetic construct
  source 1..58: mol_type = other DNA
tctgcaggcattttgcgtattttttcggcatgatggccagatagtatttactaccata

SEQ ID NO: 25 - DNA, 51 bp, synthetic construct
  source 1..51: mol_type = other DNA
acaaacgttttagcgaggatcagtatctgctgcatatcccgattgccatca

SEQ ID NO: 26 - DNA, 51 bp, synthetic construct
  source 1..51: mol_type = other DNA
tgatggcaatcgggatatgcagcagatactgatcctcgctaaaacgtttgt

SEQ ID NO: 27 - RNA, 44 nt, synthetic construct
  source 1..44: mol_type = other RNA
taatttctactaagtgtagatatagtctttccttgggtctgtta

SEQ ID NO: 28 - RNA, 44 nt, synthetic construct
  source 1..44: mol_type = other RNA
taatttctactaagtgtagatcttgggtgtgttaaaagtgacca

SEQ ID NO: 29 - RNA, 44 nt, synthetic construct
  source 1..44: mol_type = other RNA
taatttctactaagtgtagatacacacccaaggaaagactatga

SEQ ID NO: 30 - RNA, 44 nt, synthetic construct
  source 1..44: mol_type = other RNA
taatttctactaagtgtagatatccgtgctgagtgtaccatgca

SEQ ID NO: 31 - RNA, 44 nt, synthetic construct
  source 1..44: mol_type = other RNA
taatttctactaagtgtagattaaacactgtttcatttcatccg

SEQ ID NO: 32 - RNA, 44 nt, synthetic construct
  source 1..44: mol_type = other RNA
taatttctactaagtgtagatgaaacgtcagtcttctcttttgt

SEQ ID NO: 33 - RNA, 44 nt, synthetic construct
  source 1..44: mol_type = other RNA
taatttctactaagtgtagattaatgccctgtagtctctctgta

SEQ ID NO: 34 - RNA, 44 nt, synthetic construct
  source 1..44: mol_type = other RNA
taatttctactaagtgtagattaattaacagcttgctggtgaaa

SEQ ID NO: 35 - RNA, 44 nt, synthetic construct
  source 1..44: mol_type = other RNA
taatttctactaagtgtagatggttaaagatggttaaatgattg

SEQ ID NO: 36 - RNA, 44 nt, synthetic construct
  source 1..44: mol_type = other RNA
taatttctactaagtgtagattgtgaaatggcttataattgctt

SEQ ID NO: 37 - RNA, 44 nt, synthetic construct
  source 1..44: mol_type = other RNA
taatttctactaagtgtagatgttgttggatttgaaattccaga

SEQ ID NO: 38 - RNA, 44 nt, synthetic construct
  source 1..44: mol_type = other RNA
taatttctactaagtgtagatttgtaggatatgcccttgactat

SEQ ID NO: 39 - RNA, 41 nt, synthetic construct
  source 1..41: mol_type = other RNA
taatttctactcttgtagatatagtctttccttgggtgtgt

SEQ ID NO: 40 - RNA, 41 nt, synthetic construct
  source 1..41: mol_type = other RNA
taatttctactcttgtagatcttgggtgtgttaaaagtgac

SEQ ID NO: 41 - RNA, 41 nt, synthetic construct
  source 1..41: mol_type = other RNA
taatttctactcttgtagatacacacccaaggaaagactat

SEQ ID NO: 42 - RNA, 41 nt, synthetic construct
  source 1..41: mol_type = other RNA
taatttctactcttgtagatatccgtgctgagtgtaccatg

SEQ ID NO: 43 - RNA, 41 nt, synthetic construct
  source 1..41: mol_type = other RNA
taatttctactcttgtagattaaacactgtttcatttcatc

SEQ ID NO: 44 - RNA, 41 nt, synthetic construct
  source 1..41: mol_type = other RNA
taatttctactcttgtagatgaaacgtcagtcttctctttt

SEQ ID NO: 45 - RNA, 41 nt, synthetic construct
  source 1..41: mol_type = other RNA
taatttctactcttgtagattaatgccctgtagtctctctg

SEQ ID NO: 46 - RNA, 41 nt, synthetic construct
  source 1..41: mol_type = other RNA
taatttctactcttgtagattaattaacagcttgctggtga

SEQ ID NO: 47 - RNA, 41 nt, synthetic construct
  source 1..41: mol_type = other RNA
taatttctactcttgtagatggttaaagatggttaaatgat

SEQ ID NO: 48 - RNA, 41 nt, synthetic construct
  source 1..41: mol_type = other RNA
taatttctactcttgtagattgtgaaatggcttataattgc

SEQ ID NO: 49 - RNA, 41 nt, synthetic construct
  source 1..41: mol_type = other RNA
taatttctactcttgtagatgttgttggatttgaaattcca

SEQ ID NO: 50 - RNA, 41 nt, synthetic construct
  source 1..41: mol_type = other RNA
taatttctactcttgtagatttgtaggatatgcccttgact

SEQ ID NO: 51 - DNA, 26 bp, synthetic construct
  source 1..26: mol_type = other DNA
aagaagttgtgataaaaggtgatgct

SEQ ID NO: 52 - DNA, 24 bp, synthetic construct
  source 1..24: mol_type = other DNA
acacatccatgggacttctgcctc

SEQ ID NO: 53 - protein, 15 aa, synthetic construct
GLNDIFEAQKIEWHE

SEQ ID NO: 54 - protein, 26 aa, synthetic construct
KRRWKKNFIAVSAANRFKKISSSGAL

SEQ ID NO: 55 - protein, 6 aa, synthetic construct
EEEEEE

SEQ ID NO: 56 - protein, 13 aa, synthetic construct
GAPVPYPDPLEPR

SEQ ID NO: 57 - protein, 8 aa, synthetic construct
DYKDDDDK

SEQ ID NO: 58 - protein, 9 aa, synthetic construct
YPYDVPDYA

SEQ ID NO: 59 - protein, 6 aa, synthetic construct
HHHHHH

SEQ ID NO: 60 - protein, 10 aa, synthetic construct
EQKLISEEDL

SEQ ID NO: 61 - protein, 18 aa, synthetic construct
TKENPRSNQEESYDDNES

SEQ ID NO: 62 - protein, 15 aa, synthetic construct
KETAAAKFERQHMDS

SEQ ID NO: 63 - protein, 38 aa, synthetic construct
MDEKTTGWRGGHVVEGLAGELEQLRARLEHHPQGQREP

SEQ ID NO: 64 - protein, 13 aa, synthetic construct
SLAELLNAGLGGS

SEQ ID NO: 65 - protein, 8 aa, synthetic construct
TQDPSRVG

SEQ ID NO: 66 - protein, 8 aa, synthetic construct
WSHPQFEK

SEQ ID NO: 67 - protein, 6 aa, synthetic construct
CCPGCC

SEQ ID NO: 68 - protein, 14 aa, synthetic construct
GKPIPNPLLGLDST

SEQ ID NO: 69 - protein, 11 aa, synthetic construct
YTDIEMNRLGK

SEQ ID NO: 70 - protein, 8 aa, synthetic construct
DLYDDDDK

SEQ ID NO: 71 - protein, 16 aa, synthetic construct
TDKDMTITFTNKKDAE

SEQ ID NO: 72 - protein, 13 aa, synthetic construct
AHIVMVDAYKPTK

SEQ ID NO: 73 - protein, 12 aa, synthetic construct
KLGDIEFIKVNK

(19) United States
(12) Patent Application Publication
(10) Pub. No.: US 2025/0179457 A1
(43) Pub. Date: Jun. 5, 2025

(54) LACHNOSPIRACEAE BACTERIUM ND2006 CAS12A MUTANT GENES AND POLYPEPTIDES ENCODED BY SAME

(71) Applicant: INTEGRATED DNA TECHNOLOGIES, INC., Coralville, IA (US)

(72) Inventors: Sarah Franz BEAUDOIN, Iowa City, IA (US); Michael Allen COLLINGWOOD, North Liberty, IA (US); Christopher Anthony VAKULSKAS, North Liberty, IA (US)

(21) Appl. No.: 19/061,780

(22) Filed: Feb. 24, 2025

Related U.S. Application Data
(62) Division of application No. 17/430,013, filed on Aug. 11, 2021, now Pat. No. 12,264,342.
(60) Provisional application No. 62/808,984, filed on Feb. 22, 2019.

(51) Int. Cl. C12N 9/22; C12N 15/11

(52) U.S. Cl. CPC C12N 9/22; C12N 15/11; C12N 2310/20

(57) ABSTRACT

This invention pertains to mutant Lachnospiraceae bacterium ND2006 (Lb) Cas12a nucleic acids and proteins for use in CRISPR/Cas12a endonuclease systems, and their methods of use. In particular, the invention pertains to an isolated mutant LbCas12a protein, wherein the isolated mutant LbCas12a protein is active in a CRISPR/Cas12a endonuclease system. The invention also includes isolated nucleic acids encoding mutant LbCas12a proteins, ribonucleoprotein complexes and CRISPR/Cas12a endonuclease systems having mutant LbCas12a proteins.

### CROSS-REFERENCE TO RELATED APPLICATIONS

This application is a divisional of U.S. patent application Ser. No. 17/430,013, filed Aug. 11, 2021, which is a U.S. national stage entry of International Patent Application No. PCT/US2020/019168, filed on Feb. 21, 2020, which claims the benefit to U.S. Provisional Patent Application No. 62/808,984, filed on Feb. 22, 2019, each of which is incorporated by reference herein in its entirety.

### FIELD OF THE INVENTION

This invention pertains to Lachnospiraceae bacterium Cas12a based CRISPR genes, polypeptides encoded by the same, mammalian cell lines that stably express Cas 12, crRNAs and the use of these materials in compositions of CRISPR-Cas12a systems and methods.

### REFERENCE TO SEQUENCE LISTING

This application was filed with a Sequence Listing XML in ST.26 XML format in accordance with 37 C.F.R. § 1.831. The Sequence Listing XML file submitted in the USPTO Patent Center, “013670-9059-US03_sequence_listing_xml_17-FEB-2025.xml,” was created on Feb. 17, 2025, contains 73 sequences, has a file size of 116 kilobytes (118,784 bytes), and is incorporated by reference in its entirety into the specification.

### BACKGROUND

Cas12a (previously named Cpf1) is a class 2/type V CRISPR RNA-guided endonuclease. Zetsche et al., Cell 163: 1-13 (2015). Cas12a is an effective nuclease used for genome editing and is an alternative to the Cas9 enzyme. Cas12a is a ˜1300 amino acid protein and is slightly smaller than Cas9 from S. pyogenes. The Cas12 system does not utilize a separate tracrRNA, and only requires a single short crRNA of 40-45 nucleotides in length that both specifies target DNA sequence and directs binding of the RNA to the Cas12a nuclease. Hur et al., Nature Biotechnology 34: 807-808 (2016). The PAM recognition sequence of Cas12a is TTTV which allows for expanded coverage in adenine and thymidine rich areas of the genome that Cas9 cannot access.

Cleavage by Cas12a results in a staggered double-stranded break in the DNA with 4-5 nucleotide overhangs, which leaves staggered ends distal to the PAM site. Gao et al., Cell Research 26: 901-913 (2016). These double stranded breaks can then be repaired via non-homologous end joining (NHEJ) which often leads to mutations or insertions/deletions at the cut site or site or homology directed repair (HDR) which can generate precise editing events. Furthermore, when Cas12a cleaves, it does so further away from PAM than Cas9, which is also further away from the target site. As a result, the protospacer, and especially the seed sequence of the protospacer, are less likely to be edited, thereby leaving open the potential for a second round of cleavage if the desired repair event doesn't occur the first time.

LbCas12a is an RNA guided endonuclease from the Clustered Regularly Interspaced Short Palindromic Repeat (CRISPR) adaptive immune system from Lachnospiraceae bacterium ND2006 (Lb) species. Cas12a nucleases are classified as a class 2 type V CRISPR system that provide a staggered DNA double-stranded break with a 5-nucleotide 5′-overhang when complexed with a CRISPR RNA (crRNA) [1]. The LbCas12a:crRNA complex is referred to as a CRISPR ribonucleoprotein (RNP) complex.

LbCas12a, along with AsCas12a (Acidaminococcus sp. BV3L6), was first characterized in 2015 [1] and since have successfully been used for genome editing in eukaryotic cells [1-8]. The two Cas12a variants, As and Lb, share a 34% sequence identity and have both been crystallized by the Nureki group [9-10]. The RuvC and Nuc domains of both variants of Cas12a are structurally similar and cleave the target DNA by similar mechanisms [9-10]. Both variants recognize the TTTV as the canonical PAM and have been shown to tolerate CTTV, TCTV and TTCV as non-canonical PAMs [6, 10].

Engineered Cas12a proteins have been reported by Zhang and coworkers that show altered PAM specificities [11]. Their primary objective was to perform a structure guided mutagenesis screen on AsCas12a, followed by mirror mutations in LbCas12a. This resulted in two mutant variants, AsCas12a-S542R/K607R and AsCas12a-S542R/K548V/N552R, which recognized the PAMs TYCV and TATV, respectively. These mutants retained the high specificity of these CRISPR proteins and introducing these mutations into LbCas12a (G532R/K595R and G532R/K538V/T542R, respectively) resulted in similar PAM-altering specificity [11].

### SUMMARY

This invention pertains to Cas12a CRISPR genes and mutants, polypeptides encoded by the same, mammalian cell lines that stably express Cas12a and their use in compositions of CIRSPR-Cas12a systems and methods. Examples are shown employing the Cpf1 systems from Lachnospiraceae bacterium ND2006 (Lb) however this is not intended to limit scope, which extends to Cas12a homologs or orthologs isolated from other species.

Additionally, the present invention pertains to the ability to cleave double-stranded DNA of living organisms at precise locations with the CRISPR/LbCas12a nuclease system. Additionally, the present invention describes the single amino acid substitution of LbCas12a that enhances genome editing efficiency as compared to wild type Cas12a variants, LbCas12a and AsCas12a, and is claimed as LbCas12a-E795L. This invention also includes six other mutants of LbCas12a, N527R, D559P, N527R/D559P, N527R/E795L, D559P/E795L and N527R/D559P/E795L, that showed similar genome editing as wild type LbCas12a.

In a first embodiment an isolated mutant Cas12a protein is provided. The isolated mutant Cas12a protein is active in Clustered Regularly Interspaced Short Palindromic Repeats (CRISPR)/CRISPR-associated protein endonuclease system (“CRISPR/Cas12a endonuclease system”). The CRISPR/Cas12a endonuclease system maintained on-target editing activity relative to a wild-type CRISPR/Cas12a endonuclease system. In another aspect the Cas12a protein is isolated from Lachnospiraceae bacterium ND2006 (Lb).

In a second embodiment, an isolated ribonucleoprotein (RNP) complex is provided. The RNP complex includes a mutant Cas12a protein and a crRNA. The isolated ribonucleoprotein complex is active as a CRISPR/Cas12a endonuclease system, wherein the resultant CRISPR/Cas12a endonuclease system displays maintained on-target editing activity relative to a wild-type CRISPR/Cas12a endonuclease system.

In a third embodiment, an isolated nucleic acid encoding a mutant Cas12a protein is provided. The mutant Cas12a protein is active in a CRISPR/Cas12a endonuclease system, wherein the CRISPR/Cas12a endonuclease system displays maintained on-target editing activity relative to wild-type CRISPR/Cas12a endonuclease system.

In a fourth embodiment, a CRISPR/Cas12a endonuclease system is provided. The CRISPR/Cas12a endonuclease system includes a mutant Cas12a protein and a crRNA. The CRISPR/Cas12a endonuclease system displays maintained on-target editing activity relative to wild-type CRISPR/Cas12a endonuclease system.

In a fifth embodiment, a method of performing gene editing having maintained on-target editing activity is provided. The method includes the step of contacting a candidate editing DNA target site locus with an active CRISPR/Cas12a endonuclease system having a mutant Cas12 a protein complexed with an appropriate crRNA. Said interaction can occur an any context, for example, in a live animal, in live cells, or an isolated DNA in vitro.

In another embodiment the CRISPR/Cas12a endonuclease system of the present invention displays maintained on-target editing activity relative to a wild type CRISPR/Cas endonuclease system and may display reduced off-target editing activity when compared to wild type CRISPR/Cas endonuclease systems. In another aspect the CRISPR/Cas12a endonuclease system of the present invention displays maintained on-target editing activity relative to a wild type CRISPR/Cas12a endonuclease system and may display reduced off-target editing activity when compared to wild type CRISPR/Cas12a endonuclease system.

In another embodiment the CRISPR/Cas12a endonuclease system of the present invention displays maintained on-target editing activity relative to a wild type CRISPR/Cas endonuclease system and may display reduced off-target editing activity when compared to wild type CRISPR/Cas endonuclease systems. In another aspect the CRISPR/Cas12a endonuclease system of the present invention displays maintained on-target editing activity relative to a wild type CRISPR/Cas12a endonuclease system and may display reduced off-target editing activity when compared to wild type CRISPR/Cas12a endonuclease system.

### DESCRIPTION OF THE DRAWINGS

FIG. 1A-D show the primary and secondary structure alignment of AsCas12a (top) (SEQ ID NO: 18) and LbCas12a (bottom) (SEQ ID NO: 2). A-Helices are represented with squiggles, β-strands with arrows and turns with ‘TT’. Identical residues are boxed with solid red and similar residues are boxed with a blue outline.

FIG. 2 shows the editing efficiency of the LbCas12a mutants as compared to wild-type LbCas12a and AsCas12a-M537R/F870L mutant after 48 hours in HEK 293 human cells.

FIG. 3 shows the editing efficiency of LbCas12a mutants as compared to wild-type LbCas12a after 48 hours in HEK293 human cells with IDT Alt-R® Electroporation Enhancer.

FIG. 4 shows the editing efficiency of LbCas2a mutants as compared to wild-type LbCas12a after 48 hours in HEK293 human cells without IDT Alt-R® Electroporation Enhancer.

FIG. 5 shows the editing efficiency of LbCas12a mutants as compared to wild-type LBCas12a and AsCas 12a-M537R/F870L mutant after 48 hours in HEK293 human cells with IDT Alt-R® Electroporation Enhancer.

FIG. 6A shows the editing efficiency of LbCas12a wild type and E795L mutant LbCas12a as compared to AsCas12a wild type and AsCas12a-M537R/F870L mutant delivered as a 1.0 μM dose of RNP measured after 48 hours in HEK293 cells with IDT Alt-R® Electroporation Enhancer.

FIG. 6B shows the editing efficiency of LbCas12a wild type and E795L mutant LbCas12a as compared to AsCas12a wild type and AsCas 12a-M537R/F870L mutant delivered as a 0.22 μM dose of RNP measured after 48 hours in HEK293 cells with IDT Alt-R® Electroporation Enhancer.

FIG. 6C shows the editing efficiency of LbCas12a wild type and E795L mutant LbCas12a as compared to AsCas12a wild type and AsCas 12a-M537R/F870L mutant delivered as a 0.05 μM dose of RNP measured after 48 hours in HEK293 cells with IDT Alt-R® Electroporation Enhancer.

FIG. 6D shows the editing efficiency of LbCas12a wild type and E795L mutant LbCas12a as compared to AsCas12a wild type and AsCas 12a-M537R/F870L mutant delivered as a 0.01 μM dose of RNP measured after 48 hours in HEK293 cells with IDT Alt-R® Electroporation Enhancer

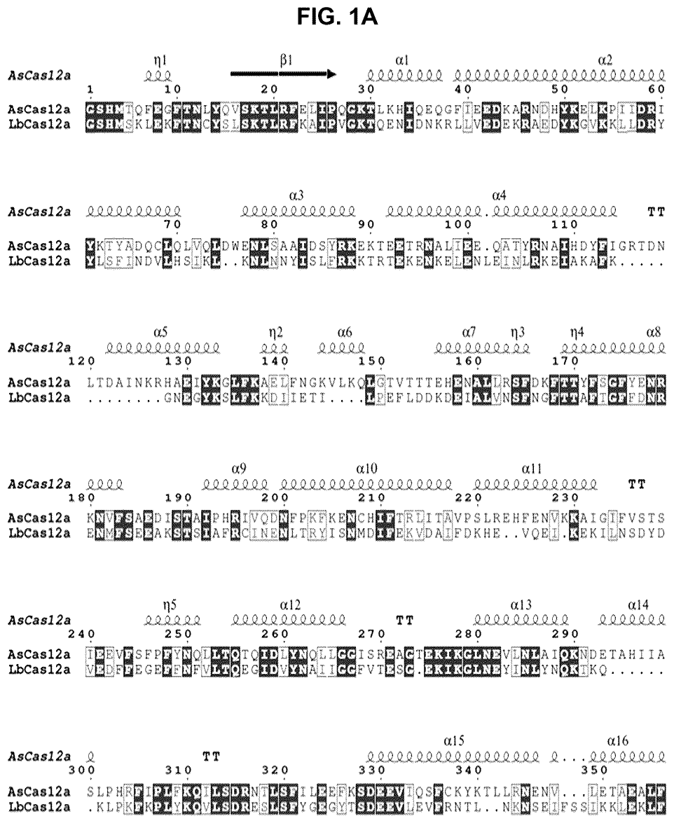

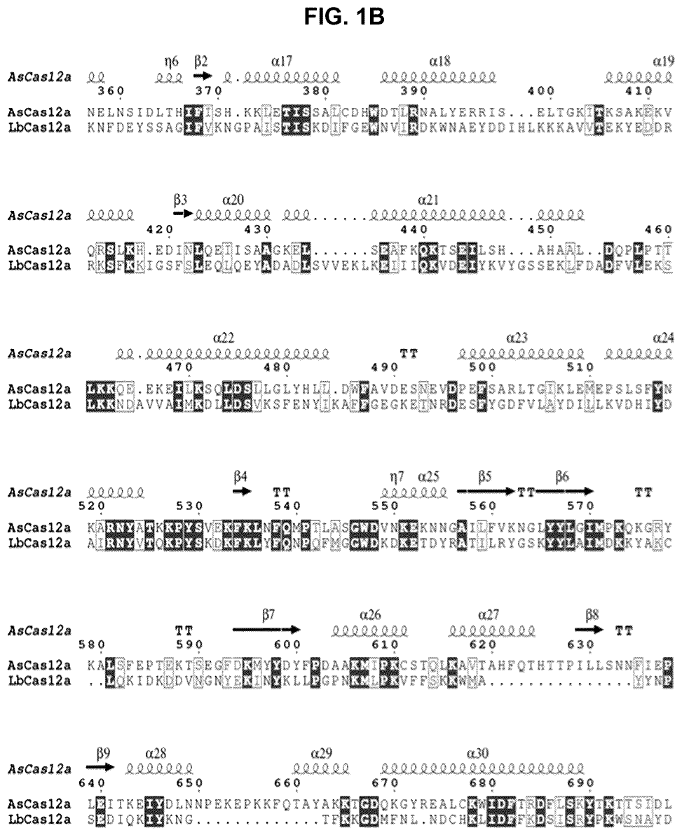

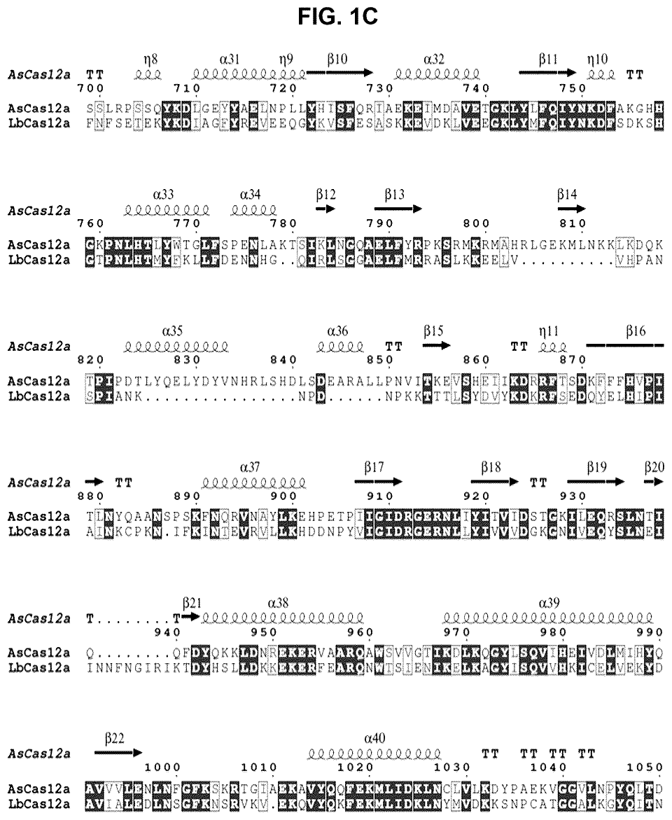

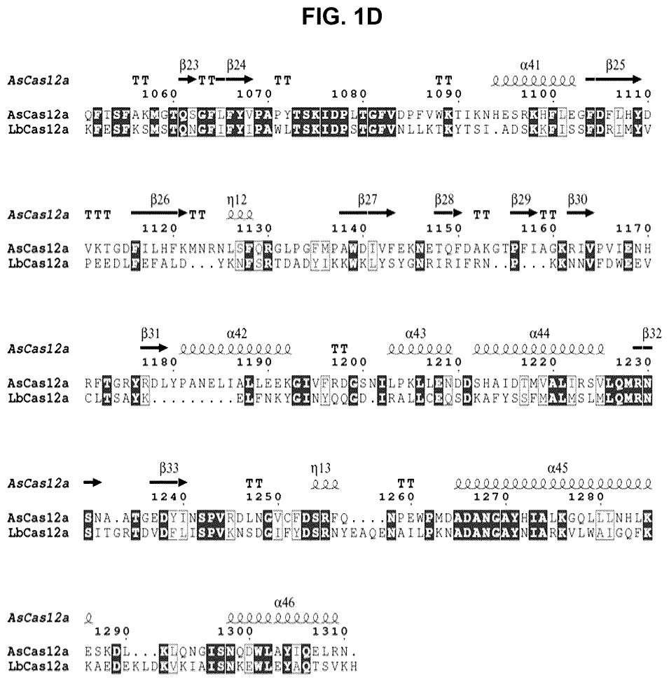

### DETAILED DESCRIPTION

The methods and compositions of the invention described herein provide mutant LbCas12a nucleic acids and polypeptides for use in a CRISPR/Cas12a system. The present invention describes novel Cas12a mutants that reduce off-target editing activity to low levels while maintaining high on-target editing activity relative to the wild-type protein even when delivered as an RNP complex. These and other advantages of the invention, as well as additional inventive features, will be apparent from the description of the invention provided herein.

Cas12a provides a useful complement to Cas9 by expanding the range of PAM sequences that can be targeted from GC-Rich areas (Cas9) to AT-rich areas of the genome (Cas12a), thereby expanding the range of sequences that can be modified using CRISPR genome engineering methods. In addition to having a T-rich PAM site, another advantage of the Cas12a system as compared with Cas9 is the use of a single short RNA molecule.

In a first embodiment an isolated mutant Cas12a protein is provided. The isolated mutant Cas12a protein is active in Clustered Regularly Interspaced Short Palindromic Repeats (CRISPR)/CRISPR-associated protein endonuclease system (“CRISPR/Cas12a endonuclease system”). The CRISPR/Cas12a endonuclease system displays maintained on-target editing activity relative to a wild-type CRISPR/Cas endonuclease system. In another aspect the Cas12a protein is isolated from Lachnospiraceae bacterium ND2006 (Lb). Preferred single mutant Cas12a proteins include substitution mutations into the WT-LbCas12a introduced at the following positions: N527, D559, and E795.

Exemplarily single mutant Cas12a proteins include the following specific mutations introduced into the WT-LbCas12a: N527R, D559P, and E795L. Exemplary single mutant Cas12a proteins include at least one-member selected form the group consisting of SEQ ID NO: 3-9. Additional substitution mutations can be included in the amino acid backgrounds of the single mutant Cas12a protein amino acid sequences, provided that the resultant Cas12a protein is active as a CRISPR/Cas12a endonuclease system, wherein the resultant CRISPR/Cas12a endonuclease system displays maintained on-target editing activity relative to a wild-type CRISPR/Cas12a endonuclease system.

Preferred multi-substitution mutant Cas12a proteins include mutations in the WT-LbCas12a introduced to at least two of the following positions: N527/D559, D559/E795, N527/E795, and N527/D559/E795. Exemplary multi-substitution mutant Cas12a proteins include mutations in the WT-LbCas12a selected from the following amino acid mutations: N527R/D559P, D559P/E795L, N527R/E795L and N527R/D559P/E795L.

Exemplary multi substitution mutant Cas12a proteins include at least one member selected from the group consisting of SEQ ID NO: 3-9. Additional substitution mutations can be included in the amino acid backgrounds of the single mutant Cas12a protein amino acid sequences, provided that the resultant Cas12a protein is active as a CRISPR/Cas12a endonuclease system, wherein the resultant CRISPR/Cas12a endonuclease system displays maintained on-target editing activity relative to a wild-type CRISPR/Cas12a endonuclease system.

In second embodiment, an isolated ribonucleoprotein complex is provided. The RNP includes mutant Cas12a protein and a crRNA complex. In one respect the crRNA includes an Alt-R® crRNA (Integrated DNA Technologies, Inc. (Coralville, IA, (US)) directed against a specific editing target site for a given locus. Preferred mutant Cas9 proteins include those as described above.

In another embodiment, an isolated nucleic acid encoding a mutant LbCas12a protein is provided. Preferred isolated nucleic acids encode mutant LbCas12a proteins as described above. Exemplary isolated nucleic acids encoding mutant LbCas12a proteins can be readily generated from a nucleic acid encoding the wild-type LbCas12a protein using recombinant DNA procedures or chemical synthesis methods. Preferred nucleic acids for this purpose include those optimized for expression of the LbCas12a proteins in bacteria, (e.g., E. coli) or mammalian (e.g., human) cells. Exemplary codon-optimized nucleic acids for expressing WT-LbCas12a in E. coli and human cells includes SEQ ID NO: 1. Moreover, the present invention contemplates fusion proteins of WT-Cas12a and mutant LbCas12a, wherein the coding sequences of WT-Cas12a and mutant LbCas12a are fused to amino acid sequences encoding for nuclear localization (“NLS”) of the fusion protein in eukaryotic cells or amino acid sequences to facilitate purification of the proteins.

In a further embodiment, the isolated nucleic acid includes mRNA encoding one of the aforementioned mutant LbCas12a proteins. In a second respect, the isolated nucleic acid includes DNA encoding a gene for one of the aforementioned mutant LbCas12a proteins. A preferred DNA includes a vector that encodes a gene encoding for a mutant LbCas12a protein. Such delivery methods include plasmid and various viral delivery vectors as are well known to those with skill in the art. The mutant LbCas12a protein can also be stably transformed into cells using suitable expression vectors to produce a cell line that constitutively or inducibly expresses the mutant LbCas12a. The aforementioned methods can also be applied to embryos to product progeny animals that constitutively or inducibly expresses the mutant LbCas12a.

In another embodiment a CRISPR/Cas12a endonuclease systems is provided.

The CRISPR/Cas12a endonuclease system includes a mutant LbCas12a protein. Preferred mutant LbCas12a proteins include those describe above. In one aspect, the CRISPR/Cas12a endonuclease system is encoded by a DNA expression vector. In one embodiment, the DNA expression vector is selected from a bacterial expression vector or a eukaryotic expression vector. In another aspect the CRISPR/Cas12a endonuclease system comprises a ribonucleoprotein complex comprising a mutant LbCas12a protein and a crRNA.

In a further embodiment, a method of performing gene editing having increased on-target editing activity is provided. The method includes the step of contacting a candidate editing target site locus with an active CRISPR/Cas12a endonuclease system having a mutant LbCas12a protein. In one aspect, the method includes a single mutant LbCas12a protein having mutations in the WT-LbCas12a introduced at one of the following positions: N527, D559, and E795. Exemplary single mutant LbCas12a proteins include the following specific mutations introduced into the WT-LbCas 12a: N527R, D559P, and E795L. Exemplary single mutant LbCas12a proteins include at least one member selected form the group consisting of SEQ ID NO: 3-9. Additional substitution mutations can be included in the amino acid backgrounds of the single mutant LbCas12a protein amino acid sequences, provided that the resultant LbCas12a protein is active as a CRISPR/Cas12a endonuclease system, wherein the resultant CRISPR/Cas12a endonuclease system displays maintained on-target editing activity relative to a wild-type CRISPR/Cas12a endonuclease system.

In another embodiment, the method includes a multi-substitution mutant LbCas12a proteins include mutations in the WT-LbCas12a introduced to at least two of the following positions: N527/D559, D559/E795, N527/E795, and N527/D559/E795. Exemplary multi-substitution mutant Cas12a proteins include mutations in the WT-LbCas12a selected from the following amino acid mutations: N527R/D559P, D559P/E795L, N527R/E795L and N527R/D559P/E795L. Exemplary multi substitution mutant LbCas12a proteins include at least one member selected from the group consisting of SEQ ID NO: 3-9. Additional substitution mutations can be included in the amino acid backgrounds of the single mutant LbCas12a protein amino acid sequences, provided that the resultant LbCas12a protein is active as a CRISPR/Cas12a endonuclease system, wherein the resultant CRISPR/Cas12a endonuclease system displays maintained on-target editing activity relative to a wild-type CRISPR/Cas12a endonuclease system.

The applications of Cas12a and LbCas12a based tools are many and varied. The applications include, but are not limited to: plant gene editing, yeast gene editing, mammalian gene editing, editing of cells in the organs of live animals, editing of embryos, rapid generation of knockout/knock-in animal lines, generating an animal model of disease state, correcting a disease state, inserting a reporter gene, and whole genome functional screening.

### EXAMPLES

### Example 1

### DNA and Amino Acid Sequences of Wild Type and Mutant LbCas12a Proteins and AsCas12a Proteins.

The list below shows different wild type (WT) and mutant Cas12a nucleases described in present invention. It will be appreciated by one with skill in the art that many different DNA sequences can encode/express the same amino acid (AA) sequence since in many cases more than one codon can encode the same amino acid. The DNA sequences shown below only serve as example and other DNA sequences that encode the same protein (e.g., same amino acid sequence) are contemplated. It is further appreciated that additional features, elements or tags may be added to said sequences, such as NLS domains and the like. Examples are shown for WT LbCas12a (Cpf1), WT AsCas12a, and mutant N527R LbCas12a, mutant D559P LbCas12a, mutant E759L LbCas12a, double mutant N527R/D559P LbCas12a, double mutant N527R/E795L LbCas12a, double mutant D559P/E795L LbCas12a, triple mutant N527R/D559P/E795L LbCas12a, and double mutant M537R/F870L AsCas12a. For LbCas12a and AsCas12a mutants only the amino acid and DNA sequences are provided, but it is contemplated that NLS domains and His-tag domains may be added to facilitate use in producing recombinant proteins for use in mammalian cells.

WT Lachnospiraceae bacterium ND2006 (Lb) Cas12a DNA sequence
SEQ ID NO: 1
ATGAGCAAACTGGAAAAGTTCACCAACTGTTATAGCCTGAGCAAAACCCTGCGTTTTAAAGCAATTCCGGTTGGTA
AAACCCAAGAGAACATTGATAATAAACGCCTGCTGGTCGAAGATGAAAAACGCGCTGAAGATTATAAAGGCGTGAA
AAAACTGCTGGATCGCTATTATCTGAGCTTCATTAACGATGTGCTGCACAGCATTAAACTGAAGAACCTGAACAAC
TATATCAGCCTGTTTCGTAAAAAAACCCGCACCGAAAAAGAAAACAAAGAGCTGGAAAACCTGGAAATCAATCTGC
GTAAAGAAATCGCCAAAGCGTTTAAAGGTAACGAGGGTTATAAAAGCCTGTTCAAGAAAGACATCATCGAAACCAT
TCTGCCGGAATTTCTGGATGATAAAGATGAAATTGCCCTGGTGAATAGCTTTAATGGCTTTACCACCGCATTTACC
GGCTTTTTTGATAATCGCGAAAACATGTTCAGCGAAGAAGCAAAAAGCACCAGCATTGCATTTCGCTGCATTAATG
AAAATCTGACCCGCTACATTAGCAACATGGATATCTTTGAAAAAGTGGACGCGATCTTCGATAAACACGAAGTGCA
AGAGATCAAAGAGAAAATCCTGAACAGCGATTATGACGTCGAAGATTTTTTTGAAGGCGAGTTCTTTAACTTCGTT
CTGACCCAAGAAGGTATCGACGTTTATAACGCAATTATTGGTGGTTTTGTTACCGAAAGCGGTGAGAAAATCAAAG
GCCTGAATGAATATATCAACCTGTATAACCAGAAAACCAAACAGAAACTGCCGAAATTCAAACCGCTGTATAAACA
GGTTCTGAGCGATCGTGAAAGCCTGAGCTTTTATGGTGAAGGTTATACCAGTGATGAAGAGGTTCTGGAAGTTTTT
CGTAACACCCTGAATAAAAACAGCGAGATCTTTAGCAGCATCAAAAAGCTTGAGAAACTGTTCAAAAACTTTGATG
AGTATAGCAGCGCAGGCATCTTTGTTAAAAATGGTCCGGCAATTAGCACCATCAGCAAAGATATTTTTGGCGAATG
GAATGTGATCCGCGATAAATGGAATGCCGAATATGATGATATCCACCTGAAAAAAAAGGCCGTGGTGACCGAGAAA
TATGAAGATGATCGTCGTAAAAGCTTCAAGAAAATTGGTAGCTTTAGCCTGGAACAGCTGCAAGAATATGCAGATG
CAGATCTGAGCGTTGTGGAAAAACTGAAAGAAATCATCATTCAGAAGGTGGACGAGATCTATAAAGTTTATGGTAG
CAGCGAAAAACTGTTCGATGCAGATTTTGTTCTGGAAAAAAGCCTGAAAAAGAATGATGCCGTTGTGGCCATTATG
AAAGATCTGCTGGATAGCGTTAAGAGCTTCGAGAATTACATCAAAGCCTTTTTTGGTGAGGGCAAAGAAACCAATC
GTGATGAAAGTTTCTATGGCGATTTTGTGCTGGCCTATGATATTCTGCTGAAAGTGGACCATATTTATGATGCCAT
TCGCAATTATGTTACCCAGAAACCGTATAGCAAAGACAAGTTCAAACTGTACTTTCAGAACCCGCAGTTTATGGGT
GGTTGGGATAAAGATAAAGAAACCGATTATCGTGCCACCATCCTGCGTTATGGTAGTAAATACTATCTGGCCATCA
TGGACAAAAAATACGCAAAATGCCTGCAGAAAATCGACAAAGATGATGTGAATGGCAACTATGAAAAAATCAACTA
CAAACTGCTGCCTGGTCCGAATAAAATGCTGCCGAAAGTGTTCTTTAGCAAGAAATGGATGGCCTATTATAACCCG
AGCGAGGATATTCAAAAGATCTACAAAAATGGCACCTTTAAAAAGGGCGACATGTTCAATCTGAACGATTGCCACA
AACTGATCGATTTCTTCAAAGATTCAATTTCGCGTTATCCGAAATGGTCCAATGCCTATGATTTTAACTTTAGCGA
AACCGAAAAATACAAAGACATTGCCGGTTTTTATCGCGAAGTGGAAGAACAGGGCTATAAAGTGAGCTTTGAAAGC
GCAAGCAAAAAAGAGGTTGATAAGCTGGTTGAAGAGGGCAAACTGTATATGTTCCAGATTTACAACAAAGATTTTA
GCGACAAAAGCCATGGCACCCCGAATCTGCATACCATGTACTTTAAACTGCTGTTCGACGAAAATAACCATGGTCA
GATTCGTCTGAGCGGTGGTGCCGAACTGTTTATGCGTCGTGCAAGTCTGAAAAAAGAAGAACTGGTTGTTCATCCG
GCAAATAGCCCGATTGCAAACAAAAATCCGGACAATCCGAAAAAAACCACGACACTGAGCTATGATGTGTATAAAG
ACAAACGTTTTAGCGAGGATCAGTATGAACTGCATATCCCGATTGCCATCAATAAATGCCCGAAAAACATCTTTAA
GATCAACACCGAAGTTCGCGTGCTGCTGAAACATGATGATAATCCGTATGTGATTGGCATTGATCGTGGTGAACGT
AACCTGCTGTATATTGTTGTTGTTGATGGTAAAGGCAACATCGTGGAACAGTATAGTCTGAACGAAATTATCAACA
ACTTTAACGGCATCCGCATCAAAACCGACTATCATAGCCTGCTGGACAAGAAAGAAAAAGAACGTTTTGAAGCACG
TCAGAACTGGACCAGTATTGAAAACATCAAAGAACTGAAAGCCGGTTATATTAGCCAGGTGGTTCATAAAATCTGT
GAGCTGGTAGAAAAATACGATGCAGTTATTGCACTGGAAGATCTGAATAGCGGTTTCAAAAATAGCCGTGTGAAAG
TCGAAAAACAGGTGTATCAGAAATTCGAGAAAATGCTGATCGACAAACTGAACTACATGGTCGACAAAAAAAGCAA
TCCGTGTGCAACCGGTGGTGCACTGAAAGGTTATCAGATTACCAACAAATTTGAAAGCTTTAAAAGCATGAGCACC
CAGAACGGCTTTATCTTCTATATTCCGGCATGGCTGACCAGCAAAATTGATCCGAGCACCGGTTTTGTGAACCTGC
TGAAAACAAAATATACCTCCATTGCCGACAGCAAGAAGTTTATTAGCAGCTTTGATCGCATTATGTATGTTCCGGA
AGAGGACCTGTTTGAATTCGCACTGGATTACAAAAATTTCAGCCGTACCGATGCCGACTACATCAAAAAATGGAAA
CTGTACAGCTATGGTAACCGCATTCGCATTTTTCGCAACCCGAAGAAAAACAATGTGTTCGATTGGGAAGAAGTTT
GTCTGACCAGCGCATATAAAGAACTTTTCAACAAATACGGCATCAACTATCAGCAGGGTGATATTCGTGCACTGCT
GTGTGAACAGAGCGATAAAGCGTTTTATAGCAGTTTTATGGCACTGATGAGCCTGATGCTGCAGATGCGTAATAGC
ATTACCGGTCGCACCGATGTGGATTTTCTGATTAGTCCGGTGAAAAATTCCGATGGCATCTTTTATGATAGCCGCA
ATTACGAAGCACAAGAAAATGCAATTCTGCCGAAAAACGCAGATGCAAATGGTGCATATAACATTGCACGTAAAGT
TCTGTGGGCAATTGGCCAGTTTAAGAAAGCAGAAGATGAGAAGCTGGACAAAGTGAAAATTGCGATCAGCAATAAA
GAGTGGCTGGAATACGCACAGACCAGCGTTAAACAT
WT LbCas12 amino acid sequence
SEQ ID NO: 2
MSKLEKFTNCYSLSKTLRFKAIPVGKTQENIDNKRLLVEDEKRAEDYKGVKKLLDRYYLSFINDVLHSIKLKNLNN
YISLFRKKTRTEKENKELENLEINLRKEIAKAFKGNEGYKSLFKKDIIETILPEFLDDKDEIALVNSFNGFTTAFT
GFFDNRENMFSEEAKSTSIAFRCINENLTRYISNMDIFEKVDAIFDKHEVQEIKEKILNSDYDVEDFFEGEFFNFV
LTQEGIDVYNAIIGGFVTESGEKIKGLNEYINLYNQKTKQKLPKFKPLYKQVLSDRESLSFYGEGYTSDEEVLEVF
RNTLNKNSEIFSSIKKLEKLFKNFDEYSSAGIFVKNGPAISTISKDIFGEWNVIRDKWNAEYDDIHLKKKAVVTEK
YEDDRRKSFKKIGSFSLEQLQEYADADLSVVEKLKEIIIQKVDEIYKVYGSSEKLFDADFVLEKSLKKNDAVVAIM
KDLLDSVKSFENYIKAFFGEGKETNRDESFYGDFVLAYDILLKVDHIYDAIRNYVTQKPYSKDKFKLYFQNPQFMG
GWDKDKETDYRATILRYGSKYYLAIMDKKYAKCLQKIDKDDVNGNYEKINYKLLPGPNKMLPKVFFSKKWMAYYNP
SEDIQKIYKNGTFKKGDMFNLNDCHKLIDFFKDSISRYPKWSNAYDFNFSETEKYKDIAGFYREVEEQGYKVSFES
ASKKEVDKLVEEGKLYMFQIYNKDFSDKSHGTPNLHTMYFKLLFDENNHGQIRLSGGAELFMRRASLKKEELVVHP
ANSPIANKNPDNPKKTTTLSYDVYKDKRFSEDQYELHIPIAINKCPKNIFKINTEVRVLLKHDDNPYVIGIDRGER
NLLYIVVVDGKGNIVEQYSLNEIINNFNGIRIKTDYHSLLDKKEKERFEARQNWTSIENIKELKAGYISQVVHKIC
ELVEKYDAVIALEDLNSGFKNSRVKVEKQVYQKFEKMLIDKLNYMVDKKSNPCATGGALKGYQITNKFESFKSMST
QNGFIFYIPAWLTSKIDPSTGFVNLLKTKYTSIADSKKFISSFDRIMYVPEEDLFEFALDYKNFSRTDADYIKKWK
LYSYGNRIRIFRNPKKNNVFDWEEVCLTSAYKELFNKYGINYQQGDIRALLCEQSDKAFYSSFMALMSLMLQMRNS
ITGRTDVDFLISPVKNSDGIFYDSRNYEAQENAILPKNADANGAYNIARKVLWAIGQFKKAEDEKLDKVKIAISNK
EWLEYAQTSVKH
Mutant N527R LbCas12a amino acid sequence
SEQ ID NO: 3
MSKLEKFTNCYSLSKTLRFKAIPVGKTQENIDNKRLLVEDEKRAEDYKGVKKLLDRYYLSFINDVLHSIKLKNLNN
YISLFRKKTRTEKENKELENLEINLRKEIAKAFKGNEGYKSLFKKDIIETILPEFLDDKDEIALVNSFNGFTTAFT
GFFDNRENMFSEEAKSTSIAFRCINENLTRYISNMDIFEKVDAIFDKHEVQEIKEKILNSDYDVEDFFEGEFFNFV
LTQEGIDVYNAIIGGFVTESGEKIKGLNEYINLYNQKTKQKLPKFKPLYKQVLSDRESLSFYGEGYTSDEEVLEVF
RNTLNKNSEIFSSIKKLEKLFKNFDEYSSAGIFVKNGPAISTISKDIFGEWNVIRDKWNAEYDDIHLKKKAVVTEK
YEDDRRKSFKKIGSFSLEQLQEYADADLSVVEKLKEIIIQKVDEIYKVYGSSEKLFDADFVLEKSLKKNDAVVAIM
KDLLDSVKSFENYIKAFFGEGKETNRDESFYGDFVLAYDILLKVDHIYDAIRNYVTQKPYSKDKFKLYFQRPQFMG
GWDKDKETDYRATILRYGSKYYLAIMDKKYAKCLQKIDKDDVNGNYEKINYKLLPGPNKMLPKVFFSKKWMAYYNP
SEDIQKIYKNGTFKKGDMFNLNDCHKLIDFFKDSISRYPKWSNAYDFNFSETEKYKDIAGFYREVEEQGYKVSFES
ASKKEVDKLVEEGKLYMFQIYNKDFSDKSHGTPNLHTMYFKLLFDENNHGQIRLSGGAELFMRRASLKKEELVVHP
ANSPIANKNPDNPKKTTTLSYDVYKDKRFSEDQYELHIPIAINKCPKNIFKINTEVRVLLKHDDNPYVIGIDRGER
NLLYIVVVDGKGNIVEQYSLNEIINNFNGIRIKTDYHSLLDKKEKERFEARQNWTSIENIKELKAGYISQVVHKIC
ELVEKYDAVIALEDLNSGFKNSRVKVEKQVYQKFEKMLIDKLNYMVDKKSNPCATGGALKGYQITNKFESFKSMST
QNGFIFYIPAWLTSKIDPSTGFVNLLKTKYTSIADSKKFISSFDRIMYVPEEDLFEFALDYKNFSRTDADYIKKWK
LYSYGNRIRIFRNPKKNNVFDWEEVCLTSAYKELFNKYGINYQQGDIRALLCEQSDKAFYSSFMALMSLMLQMRNS
ITGRTDVDFLISPVKNSDGIFYDSRNYEAQENAILPKNADANGAYNIARKVLWAIGQFKKAEDEKLDKVKIAISNK
EWLEYAQTSVKH
Mutant D559P LbCas12a amino acid sequence
SEQ ID NO: 4
MSKLEKFTNCYSLSKTLRFKAIPVGKTQENIDNKRLLVEDEKRAEDYKGVKKLLDRYYLSFINDVLHSIKLKNLNN
YISLFRKKTRTEKENKELENLEINLRKEIAKAFKGNEGYKSLFKKDIIETILPEFLDDKDEIALVNSFNGFTTAFT
GFFDNRENMFSEEAKSTSIAFRCINENLTRYISNMDIFEKVDAIFDKHEVQEIKEKILNSDYDVEDFFEGEFFNFV
LTQEGIDVYNAIIGGFVTESGEKIKGLNEYINLYNQKTKQKLPKFKPLYKQVLSDRESLSFYGEGYTSDEEVLEVF
RNTLNKNSEIFSSIKKLEKLFKNFDEYSSAGIFVKNGPAISTISKDIFGEWNVIRDKWNAEYDDIHLKKKAVVTEK
YEDDRRKSFKKIGSFSLEQLQEYADADLSVVEKLKEIIIQKVDEIYKVYGSSEKLFDADFVLEKSLKKNDAVVAIM
KDLLDSVKSFENYIKAFFGEGKETNRDESFYGDFVLAYDILLKVDHIYDAIRNYVTQKPYSKDKFKLYFQNPQFMG
GWDKDKETDYRATILRYGSKYYLAIMPKKYAKCLQKIDKDDVNGNYEKINYKLLPGPNKMLPKVFFSKKWMAYYNP
SEDIQKIYKNGTFKKGDMFNLNDCHKLIDFFKDSISRYPKWSNAYDFNFSETEKYKDIAGFYREVEEQGYKVSFES
ASKKEVDKLVEEGKLYMFQIYNKDFSDKSHGTPNLHTMYFKLLFDENNHGQIRLSGGAELFMRRASLKKEELVVHP
ANSPIANKNPDNPKKTTTLSYDVYKDKRFSEDQYELHIPIAINKCPKNIFKINTEVRVLLKHDDNPYVIGIDRGER
NLLYIVVVDGKGNIVEQYSLNEIINNFNGIRIKTDYHSLLDKKEKERFEARQNWTSIENIKELKAGYISQVVHKIC
ELVEKYDAVIALEDLNSGFKNSRVKVEKQVYQKFEKMLIDKLNYMVDKKSNPCATGGALKGYQITNKFESFKSMST
QNGFIFYIPAWLTSKIDPSTGFVNLLKTKYTSIADSKKFISSFDRIMYVPEEDLFEFALDYKNFSRTDADYIKKWK
LYSYGNRIRIFRNPKKNNVFDWEEVCLTSAYKELFNKYGINYQQGDIRALLCEQSDKAFYSSFMALMSLMLQMRNS
ITGRTDVDFLISPVKNSDGIFYDSRNYEAQENAILPKNADANGAYNIARKVLWAIGQFKKAEDEKLDKVKIAISNK
EWLEYAQTSVKH
Mutant E795L LbCas12a amino acid sequence
SEQ ID NO: 5
MSKLEKFTNCYSLSKTLRFKAIPVGKTQENIDNKRLLVEDEKRAEDYKGVKKLLDRYYLSFINDVLHSIKLKNLNN
YISLFRKKTRTEKENKELENLEINLRKEIAKAFKGNEGYKSLFKKDIIETILPEFLDDKDEIALVNSFNGFTTAFT
GFFDNRENMFSEEAKSTSIAFRCINENLTRYISNMDIFEKVDAIFDKHEVQEIKEKILNSDYDVEDFFEGEFFNFV
LTQEGIDVYNAIIGGFVTESGEKIKGLNEYINLYNQKTKQKLPKFKPLYKQVLSDRESLSFYGEGYTSDEEVLEVF
RNTLNKNSEIFSSIKKLEKLFKNFDEYSSAGIFVKNGPAISTISKDIFGEWNVIRDKWNAEYDDIHLKKKAVVTEK
YEDDRRKSFKKIGSFSLEQLQEYADADLSVVEKLKEIIIQKVDEIYKVYGSSEKLFDADFVLEKSLKKNDAVVAIM
KDLLDSVKSFENYIKAFFGEGKETNRDESFYGDFVLAYDILLKVDHIYDAIRNYVTQKPYSKDKFKLYFQNPQFMG
GWDKDKETDYRATILRYGSKYYLAIMDKKYAKCLQKIDKDDVNGNYEKINYKLLPGPNKMLPKVFFSKKWMAYYNP
SEDIQKIYKNGTFKKGDMFNLNDCHKLIDFFKDSISRYPKWSNAYDFNFSETEKYKDIAGFYREVEEQGYKVSFES
ASKKEVDKLVEEGKLYMFQIYNKDFSDKSHGTPNLHTMYFKLLFDENNHGQIRLSGGAELFMRRASLKKEELVVHP
ANSPIANKNPDNPKKTTTLSYDVYKDKRFSEDQYLLHIPIAINKCPKNIFKINTEVRVLLKHDDNPYVIGIDRGER
NLLYIVVVDGKGNIVEQYSLNEIINNFNGIRIKTDYHSLLDKKEKERFEARQNWTSIENIKELKAGYISQVVHKIC
ELVEKYDAVIALEDLNSGFKNSRVKVEKQVYQKFEKMLIDKLNYMVDKKSNPCATGGALKGYQITNKFESFKSMST
QNGFIFYIPAWLTSKIDPSTGFVNLLKTKYTSIADSKKFISSFDRIMYVPEEDLFEFALDYKNFSRTDADYIKKWK
LYSYGNRIRIFRNPKKNNVFDWEEVCLTSAYKELFNKYGINYQQGDIRALLCEQSDKAFYSSFMALMSLMLQMRNS
ITGRTDVDFLISPVKNSDGIFYDSRNYEAQENAILPKNADANGAYNIARKVLWAIGQFKKAEDEKLDKVKIAISNK
EWLEYAQTSVKH
Mutant N527R/D559P LbCas12a amino acid sequence
SEQ ID NO: 6
MSKLEKFTNCYSLSKTLRFKAIPVGKTQENIDNKRLLVEDEKRAEDYKGVKKLLDRYYLSFINDVLHSIKLKNLNNYISLF
RKKTRTEKENKELENLEINLRKEIAKAFKGNEGYKSLFKKDIIETILPEFLDDKDEIALVNSFNGFTTAFTGFFDN
RENMFSEEAKSTSIAFRCINENLTRYISNMDIFEKVDAIFDKHEVQEIKEKILNSDYDVEDFFEGEFFNFVLTQEG
IDVYNAIIGGFVTESGEKIKGLNEYINLYNQKTKQKLPKFKPLYKQVLSDRESLSFYGEGYTSDEEVLEVFRNTLN
KNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLD
SVKSFENYIKAFFGEGKETNRDESFYGDFVLAYDILLKVDHIYDAIRNYVTQKPYSKDKFKLYFQRPQFMGGWDKD
KETDYRATILRYGSKYYLAIMPKKYAKCLQKIDKDDVNGNYEKINYKLLPGPNKMLPKVFFSKKWMAYYNPSEDIQ
KIYKNGTFKKGDMFNLNDCHKLIDFFKDSISRYPKWSNAYDFNFSETEKYKDIAGFYREVEEQGYKVSFESASKKE
VDKLVEEGKLYMFQIYNKDFSDKSHGTPNLHTMYFKLLFDENNHGQIRLSGGAELFMRRASLKKEELVVHPANSPI
ANKNPDNPKKTTTLSYDVYKDKRFSEDQYELHIPIAINKCPKNIFKINTEVRVLLKHDDNPYVIGIDRGERNLLYI
VVVDGKGNIVEQYSLNEIINNFNGIRIKTDYHSLLDKKEKERFEARQNWTSIENIKELKAGYISQVVHKICELVEK
YDAVIALEDLNSGFKNSRVKVEKQVYQKFEKMLIDKLNYMVDKKSNPCATGGALKGYQITNKFESFKSMSTQNGFI
FYIPAWLTSKIDPSTGFVNLLKTKYTSIADSKKFISSFDRIMYVPEEDLFEFALDYKNFSRTDADYIKKWKLYSYG
NRIRIFRNPKKNNVFDWEEVCLTSAYKELFNKYGINYQQGDIRALLCEQSDKAFYSSFMALMSLMLQMRNSITGRT
DVDFLISPVKNSDGIFYDSRNYEAQENAILPKNADANGAYNIARKVLWAIGQFKKAEDEKLDKVKIAISNKEWLEY
AQTSVKH
Mutant 527R/E795L LbCas12a amino acid sequence
SEQ ID NO: 7
MSKLEKFTNCYSLSKTLRFKAIPVGKTQENIDNKRLLVEDEKRAEDYKGVKKLLDRYYLSFINDVLHSIKLKNLNN
YISLFRKKTRTEKENKELENLEINLRKEIAKAFKGNEGYKSLFKKDIIETILPEFLDDKDEIALVNSFNGFTTAFT
GFFDNRENMFSEEAKSTSIAFRCINENLTRYISNMDIFEKVDAIFDKHEVQEIKEKILNSDYDVEDFFEGEFFNFV
LTQEGIDVYNAIIGGFVTESGEKIKGLNEYINLYNQKTKQKLPKFKPLYKQVLSDRESLSFYGEGYTSDEEVLEVF
RNTLNKNSEIFSSIKKLEKLFKNFDEYSSAGIFVKNGPAISTISKDIFGEWNVIRDKWNAEYDDIHLKKKAVVTEK
YEDDRRKSFKKIGSFSLEQLQEYADADLSVVEKLKEIIIQKVDEIYKVYGSSEKLFDADFVLEKSLKKNDAVVAIM
KDLLDSVKSFENYIKAFFGEGKETNRDESFYGDFVLAYDILLKVDHIYDAIRNYVTQKPYSKDKFKLYFQRPQFMG
GWDKDKETDYRATILRYGSKYYLAIMDKKYAKCLQKIDKDDVNGNYEKINYKLLPGPNKMLPKVFFSKKWMAYYNP
SEDIQKIYKNGTFKKGDMFNLNDCHKLIDFFKDSISRYPKWSNAYDFNFSETEKYKDIAGFYREVEEQGYKVSFES
ASKKEVDKLVEEGKLYMFQIYNKDFSDKSHGTPNLHTMYFKLLFDENNHGQIRLSGGAELFMRRASLKKEELVVHP
ANSPIANKNPDNPKKTTTLSYDVYKDKRFSEDQYLLHIPIAINKCPKNIFKINTEVRVLLKHDDNPYVIGIDRGER
NLLYIVVVDGKGNIVEQYSLNEIINNFNGIRIKTDYHSLLDKKEKERFEARQNWTSIENIKELKAGYISQVVHKIC
ELVEKYDAVIALEDLNSGFKNSRVKVEKQVYQKFEKMLIDKLNYMVDKKSNPCATGGALKGYQITNKFESFKSMST
QNGFIFYIPAWLTSKIDPSTGFVNLLKTKYTSIADSKKFISSFDRIMYVPEEDLFEFALDYKNFSRTDADYIKKWK
LYSYGNRIRIFRNPKKNNVFDWEEVCLTSAYKELFNKYGINYQQGDIRALLCEQSDKAFYSSFMALMSLMLQMRNS
ITGRTDVDFLISPVKNSDGIFYDSRNYEAQENAILPKNADANGAYNIARKVLWAIGQFKKAEDEKLDKVKIAISNK
EWLEYAQTSVKH
Mutant D559P/E795L LbCas12a amino acid sequence
SEQ ID NO: 8
MSKLEKFTNCYSLSKTLRFKAIPVGKTQENIDNKRLLVEDEKRAEDYKGVKKLLDRYYLSFINDVLHSIKLKNLNN
YISLFRKKTRTEKENKELENLEINLRKEIAKAFKGNEGYKSLFKKDIIETILPEFLDDKDEIALVNSFNGFTTAFT
GFFDNRENMFSEEAKSTSIAFRCINENLTRYISNMDIFEKVDAIFDKHEVQEIKEKILNSDYDVEDFFEGEFFNFV
LTQEGIDVYNAIIGGFVTESGEKIKGLNEYINLYNQKTKQKLPKFKPLYKQVLSDRESLSFYGEGYTSDEEVLEVF
RNTLNKNSEIFSSIKKLEKLFKNFDEYSSAGIFVKNGPAISTISKDIFGEWNVIRDKWNAEYDDIHLKKKAVVTEK
YEDDRRKSFKKIGSFSLEQLQEYADADLSVVEKLKEIIIQKVDEIYKVYGSSEKLFDADFVLEKSLKKNDAVVAIM
KDLLDSVKSFENYIKAFFGEGKETNRDESFYGDFVLAYDILLKVDHIYDAIRNYVTQKPYSKDKFKLYFQNPQFMG
GWDKDKETDYRATILRYGSKYYLAIMPKKYAKCLQKIDKDDVNGNYEKINYKLLPGPNKMLPKVFFSKKWMAYYNP
SEDIQKIYKNGTFKKGDMFNLNDCHKLIDFFKDSISRYPKWSNAYDFNFSETEKYKDIAGFYREVEEQGYKVSFES
ASKKEVDKLVEEGKLYMFQIYNKDFSDKSHGTPNLHTMYFKLLFDENNHGQIRLSGGAELFMRRASLKKEELVVHP
ANSPIANKNPDNPKKTTTLSYDVYKDKRFSEDQYLLHIPIAINKCPKNIFKINTEVRVLLKHDDNPYVIGIDRGER
NLLYIVVVDGKGNIVEQYSLNEIINNFNGIRIKTDYHSLLDKKEKERFEARQNWTSIENIKELKAGYISQVVHKIC
ELVEKYDAVIALEDLNSGFKNSRVKVEKQVYQKFEKMLIDKLNYMVDKKSNPCATGGALKGYQITNKFESFKSMST
QNGFIFYIPAWLTSKIDPSTGFVNLLKTKYTSIADSKKFISSFDRIMYVPEEDLFEFALDYKNFSRTDADYIKKWK
LYSYGNRIRIFRNPKKNNVFDWEEVCLTSAYKELFNKYGINYQQGDIRALLCEQSDKAFYSSFMALMSLMLQMRNS
ITGRTDVDFLISPVKNSDGIFYDSRNYEAQENAILPKNADANGAYNIARKVLWAIGQFKKAEDEKLDKVKIAISNK
EWLEYAQTSVKH
Mutant N527R/D559P/E795L LbCas12a amino acid sequence
SEQ ID NO: 9
MSKLEKFTNCYSLSKTLRFKAIPVGKTQENIDNKRLLVEDEKRAEDYKGVKKLLDRYYLSFINDVLHSIKLKNLNN
YISLFRKKTRTEKENKELENLEINLRKEIAKAFKGNEGYKSLFKKDIIETILPEFLDDKDEIALVNSFNGFTTAFT
GFFDNRENMFSEEAKSTSIAFRCINENLTRYISNMDIFEKVDAIFDKHEVQEIKEKILNSDYDVEDFFEGEFFNFV
LTQEGIDVYNAIIGGFVTESGEKIKGLNEYINLYNQKTKQKLPKFKPLYKQVLSDRESLSFYGEGYTSDEEVLEVF
RNTLNKNSEIFSSIKKLEKLFKNFDEYSSAGIFVKNGPAISTISKDIFGEWNVIRDKWNAEYDDIHLKKKAVVTEK
YEDDRRKSFKKIGSFSLEQLQEYADADLSVVEKLKEIIIQKVDEIYKVYGSSEKLFDADFVLEKSLKKNDAVVAIM
KDLLDSVKSFENYIKAFFGEGKETNRDESFYGDFVLAYDILLKVDHIYDAIRNYVTQKPYSKDKFKLYFQRPQFMG
GWDKDKETDYRATILRYGSKYYLAIMPKKYAKCLQKIDKDDVNGNYEKINYKLLPGPNKMLPKVFFSKKWMAYYNP
SEDIQKIYKNGTFKKGDMFNLNDCHKLIDFFKDSISRYPKWSNAYDFNFSETEKYKDIAGFYREVEEQGYKVSFES
ASKKEVDKLVEEGKLYMFQIYNKDFSDKSHGTPNLHTMYFKLLFDENNHGQIRLSGGAELFMRRASLKKEELVVHP
ANSPIANKNPDNPKKTTTLSYDVYKDKRFSEDQYLLHIPIAINKCPKNIFKINTEVRVLLKHDDNPYVIGIDRGER
NLLYIVVVDGKGNIVEQYSLNEIINNFNGIRIKTDYHSLLDKKEKERFEARQNWTSIENIKELKAGYISQVVHKIC
ELVEKYDAVIALEDLNSGFKNSRVKVEKQVYQKFEKMLIDKLNYMVDKKSNPCATGGALKGYQITNKFESFKSMST
QNGFIFYIPAWLTSKIDPSTGFVNLLKTKYTSIADSKKFISSFDRIMYVPEEDLFEFALDYKNFSRTDADYIKKWK
LYSYGNRIRIFRNPKKNNVFDWEEVCLTSAYKELFNKYGINYQQGDIRALLCEQSDKAFYSSFMALMSLMLQMRNS
ITGRTDVDFLISPVKNSDGIFYDSRNYEAQENAILPKNADANGAYNIARKVLWAIGQFKKAEDEKLDKVKIAISNK
EWLEYAQTSVKH
Mutant N527R LbCas12a DNA sequence
SEQ ID NO: 10
ATGAGCAAACTGGAAAAGTTCACCAACTGTTATAGCCTGAGCAAAACCCTGCGTTTTAAAGCAATTCCGGTTGGTA
AAACCCAAGAGAACATTGATAATAAACGCCTGCTGGTCGAAGATGAAAAACGCGCTGAAGATTATAAAGGCGTGAA
AAAACTGCTGGATCGCTATTATCTGAGCTTCATTAACGATGTGCTGCACAGCATTAAACTGAAGAACCTGAACAAC
TATATCAGCCTGTTTCGTAAAAAAACCCGCACCGAAAAAGAAAACAAAGAGCTGGAAAACCTGGAAATCAATCTGC
GTAAAGAAATCGCCAAAGCGTTTAAAGGTAACGAGGGTTATAAAAGCCTGTTCAAGAAAGACATCATCGAAACCAT
TCTGCCGGAATTTCTGGATGATAAAGATGAAATTGCCCTGGTGAATAGCTTTAATGGCTTTACCACCGCATTTACC
GGCTTTTTTGATAATCGCGAAAACATGTTCAGCGAAGAAGCAAAAAGCACCAGCATTGCATTTCGCTGCATTAATG
AAAATCTGACCCGCTACATTAGCAACATGGATATCTTTGAAAAAGTGGACGCGATCTTCGATAAACACGAAGTGCA
AGAGATCAAAGAGAAAATCCTGAACAGCGATTATGACGTCGAAGATTTTTTTGAAGGCGAGTTCTTTAACTTCGTT
CTGACCCAAGAAGGTATCGACGTTTATAACGCAATTATTGGTGGTTTTGTTACCGAAAGCGGTGAGAAAATCAAAG
GCCTGAATGAATATATCAACCTGTATAACCAGAAAACCAAACAGAAACTGCCGAAATTCAAACCGCTGTATAAACA
GGTTCTGAGCGATCGTGAAAGCCTGAGCTTTTATGGTGAAGGTTATACCAGTGATGAAGAGGTTCTGGAAGTTTTT
CGTAACACCCTGAATAAAAACAGCGAGATCTTTAGCAGCATCAAAAAGCTTGAGAAACTGTTCAAAAACTTTGATG
AGTATAGCAGCGCAGGCATCTTTGTTAAAAATGGTCCGGCAATTAGCACCATCAGCAAAGATATTTTTGGCGAATG
GAATGTGATCCGCGATAAATGGAATGCCGAATATGATGATATCCACCTGAAAAAAAAGGCCGTGGTGACCGAGAAA
TATGAAGATGATCGTCGTAAAAGCTTCAAGAAAATTGGTAGCTTTAGCCTGGAACAGCTGCAAGAATATGCAGATG
CAGATCTGAGCGTTGTGGAAAAACTGAAAGAAATCATCATTCAGAAGGTGGACGAGATCTATAAAGTTTATGGTAG
CAGCGAAAAACTGTTCGATGCAGATTTTGTTCTGGAAAAAAGCCTGAAAAAGAATGATGCCGTTGTGGCCATTATG
AAAGATCTGCTGGATAGCGTTAAGAGCTTCGAGAATTACATCAAAGCCTTTTTTGGTGAGGGCAAAGAAACCAATC
GTGATGAAAGTTTCTATGGCGATTTTGTGCTGGCCTATGATATTCTGCTGAAAGTGGACCATATTTATGATGCCAT
TCGCAATTATGTTACCCAGAAACCGTATAGCAAAGACAAGTTCAAACTGTACTTTCAGCGTCCGCAGTTTATGGGT
GGTTGGGATAAAGATAAAGAAACCGATTATCGTGCCACCATCCTGCGTTATGGTAGTAAATACTATCTGGCCATCA
TGGACAAAAAATACGCAAAATGCCTGCAGAAAATCGACAAAGATGATGTGAATGGCAACTATGAAAAAATCAACTA
CAAACTGCTGCCTGGTCCGAATAAAATGCTGCCGAAAGTGTTCTTTAGCAAGAAATGGATGGCCTATTATAACCCG
AGCGAGGATATTCAAAAGATCTACAAAAATGGCACCTTTAAAAAGGGCGACATGTTCAATCTGAACGATTGCCACA
AACTGATCGATTTCTTCAAAGATTCAATTTCGCGTTATCCGAAATGGTCCAATGCCTATGATTTTAACTTTAGCGA
AACCGAAAAATACAAAGACATTGCCGGTTTTTATCGCGAAGTGGAAGAACAGGGCTATAAAGTGAGCTTTGAAAGC
GCAAGCAAAAAAGAGGTTGATAAGCTGGTTGAAGAGGGCAAACTGTATATGTTCCAGATTTACAACAAAGATTTTA
GCGACAAAAGCCATGGCACCCCGAATCTGCATACCATGTACTTTAAACTGCTGTTCGACGAAAATAACCATGGTCA
GATTCGTCTGAGCGGTGGTGCCGAACTGTTTATGCGTCGTGCAAGTCTGAAAAAAGAAGAACTGGTTGTTCATCCG
GCAAATAGCCCGATTGCAAACAAAAATCCGGACAATCCGAAAAAAACCACGACACTGAGCTATGATGTGTATAAAG
ACAAACGTTTTAGCGAGGATCAGTATGAACTGCATATCCCGATTGCCATCAATAAATGCCCGAAAAACATCTTTAA
GATCAACACCGAAGTTCGCGTGCTGCTGAAACATGATGATAATCCGTATGTGATTGGCATTGATCGTGGTGAACGT
AACCTGCTGTATATTGTTGTTGTTGATGGTAAAGGCAACATCGTGGAACAGTATAGTCTGAACGAAATTATCAACA
ACTTTAACGGCATCCGCATCAAAACCGACTATCATAGCCTGCTGGACAAGAAAGAAAAAGAACGTTTTGAAGCACG
TCAGAACTGGACCAGTATTGAAAACATCAAAGAACTGAAAGCCGGTTATATTAGCCAGGTGGTTCATAAAATCTGT
GAGCTGGTAGAAAAATACGATGCAGTTATTGCACTGGAAGATCTGAATAGCGGTTTCAAAAATAGCCGTGTGAAAG
TCGAAAAACAGGTGTATCAGAAATTCGAGAAAATGCTGATCGACAAACTGAACTACATGGTCGACAAAAAAAGCAA
TCCGTGTGCAACCGGTGGTGCACTGAAAGGTTATCAGATTACCAACAAATTTGAAAGCTTTAAAAGCATGAGCACC
CAGAACGGCTTTATCTTCTATATTCCGGCATGGCTGACCAGCAAAATTGATCCGAGCACCGGTTTTGTGAACCTGC
TGAAAACAAAATATACCTCCATTGCCGACAGCAAGAAGTTTATTAGCAGCTTTGATCGCATTATGTATGTTCCGGA
AGAGGACCTGTTTGAATTCGCACTGGATTACAAAAATTTCAGCCGTACCGATGCCGACTACATCAAAAAATGGAAA
CTGTACAGCTATGGTAACCGCATTCGCATTTTTCGCAACCCGAAGAAAAACAATGTGTTCGATTGGGAAGAAGTTT
GTCTGACCAGCGCATATAAAGAACTTTTCAACAAATACGGCATCAACTATCAGCAGGGTGATATTCGTGCACTGCT
GTGTGAACAGAGCGATAAAGCGTTTTATAGCAGTTTTATGGCACTGATGAGCCTGATGCTGCAGATGCGTAATAGC
ATTACCGGTCGCACCGATGTGGATTTTCTGATTAGTCCGGTGAAAAATTCCGATGGCATCTTTTATGATAGCCGCA
ATTACGAAGCACAAGAAAATGCAATTCTGCCGAAAAACGCAGATGCAAATGGTGCATATAACATTGCACGTAAAGT
TCTGTGGGCAATTGGCCAGTTTAAGAAAGCAGAAGATGAGAAGCTGGACAAAGTGAAAATTGCGATCAGCAATAAA
GAGTGGCTGGAATACGCACAGACCAGCGTTAAACAT
Mutant D559P LbCas12a DNA sequence
SEQ ID NO: 11
ATGAGCAAACTGGAAAAGTTCACCAACTGTTATAGCCTGAGCAAAACCCTGCGTTTTAAAGCAATTCCGGTTGGTA
AAACCCAAGAGAACATTGATAATAAACGCCTGCTGGTCGAAGATGAAAAACGCGCTGAAGATTATAAAGGCGTGAA
AAAACTGCTGGATCGCTATTATCTGAGCTTCATTAACGATGTGCTGCACAGCATTAAACTGAAGAACCTGAACAAC
TATATCAGCCTGTTTCGTAAAAAAACCCGCACCGAAAAAGAAAACAAAGAGCTGGAAAACCTGGAAATCAATCTGC
GTAAAGAAATCGCCAAAGCGTTTAAAGGTAACGAGGGTTATAAAAGCCTGTTCAAGAAAGACATCATCGAAACCAT
TCTGCCGGAATTTCTGGATGATAAAGATGAAATTGCCCTGGTGAATAGCTTTAATGGCTTTACCACCGCATTTACC
GGCTTTTTTGATAATCGCGAAAACATGTTCAGCGAAGAAGCAAAAAGCACCAGCATTGCATTTCGCTGCATTAATG
AAAATCTGACCCGCTACATTAGCAACATGGATATCTTTGAAAAAGTGGACGCGATCTTCGATAAACACGAAGTGCA
AGAGATCAAAGAGAAAATCCTGAACAGCGATTATGACGTCGAAGATTTTTTTGAAGGCGAGTTCTTTAACTTCGTT
CTGACCCAAGAAGGTATCGACGTTTATAACGCAATTATTGGTGGTTTTGTTACCGAAAGCGGTGAGAAAATCAAAG
GCCTGAATGAATATATCAACCTGTATAACCAGAAAACCAAACAGAAACTGCCGAAATTCAAACCGCTGTATAAACA
GGTTCTGAGCGATCGTGAAAGCCTGAGCTTTTATGGTGAAGGTTATACCAGTGATGAAGAGGTTCTGGAAGTTTTT
CGTAACACCCTGAATAAAAACAGCGAGATCTTTAGCAGCATCAAAAAGCTTGAGAAACTGTTCAAAAACTTTGATG
AGTATAGCAGCGCAGGCATCTTTGTTAAAAATGGTCCGGCAATTAGCACCATCAGCAAAGATATTTTTGGCGAATG
GAATGTGATCCGCGATAAATGGAATGCCGAATATGATGATATCCACCTGAAAAAAAAGGCCGTGGTGACCGAGAAA
TATGAAGATGATCGTCGTAAAAGCTTCAAGAAAATTGGTAGCTTTAGCCTGGAACAGCTGCAAGAATATGCAGATG
CAGATCTGAGCGTTGTGGAAAAACTGAAAGAAATCATCATTCAGAAGGTGGACGAGATCTATAAAGTTTATGGTAG
CAGCGAAAAACTGTTCGATGCAGATTTTGTTCTGGAAAAAAGCCTGAAAAAGAATGATGCCGTTGTGGCCATTATG
AAAGATCTGCTGGATAGCGTTAAGAGCTTCGAGAATTACATCAAAGCCTTTTTTGGTGAGGGCAAAGAAACCAATC
GTGATGAAAGTTTCTATGGCGATTTTGTGCTGGCCTATGATATTCTGCTGAAAGTGGACCATATTTATGATGCCAT
TCGCAATTATGTTACCCAGAAACCGTATAGCAAAGACAAGTTCAAACTGTACTTTCAGAACCCGCAGTTTATGGGT
GGTTGGGATAAAGATAAAGAAACCGATTATCGTGCCACCATCCTGCGTTATGGTAGTAAATACTATCTGGCCATCA
TGCCGAAAAAATACGCAAAATGCCTGCAGAAAATCGACAAAGATGATGTGAATGGCAACTATGAAAAAATCAACTA
CAAACTGCTGCCTGGTCCGAATAAAATGCTGCCGAAAGTGTTCTTTAGCAAGAAATGGATGGCCTATTATAACCCG
AGCGAGGATATTCAAAAGATCTACAAAAATGGCACCTTTAAAAAGGGCGACATGTTCAATCTGAACGATTGCCACA
AACTGATCGATTTCTTCAAAGATTCAATTTCGCGTTATCCGAAATGGTCCAATGCCTATGATTTTAACTTTAGCGA
AACCGAAAAATACAAAGACATTGCCGGTTTTTATCGCGAAGTGGAAGAACAGGGCTATAAAGTGAGCTTTGAAAGC
GCAAGCAAAAAAGAGGTTGATAAGCTGGTTGAAGAGGGCAAACTGTATATGTTCCAGATTTACAACAAAGATTTTA
GCGACAAAAGCCATGGCACCCCGAATCTGCATACCATGTACTTTAAACTGCTGTTCGACGAAAATAACCATGGTCA
GATTCGTCTGAGCGGTGGTGCCGAACTGTTTATGCGTCGTGCAAGTCTGAAAAAAGAAGAACTGGTTGTTCATCCG
GCAAATAGCCCGATTGCAAACAAAAATCCGGACAATCCGAAAAAAACCACGACACTGAGCTATGATGTGTATAAAG
ACAAACGTTTTAGCGAGGATCAGTATGAACTGCATATCCCGATTGCCATCAATAAATGCCCGAAAAACATCTTTAA
GATCAACACCGAAGTTCGCGTGCTGCTGAAACATGATGATAATCCGTATGTGATTGGCATTGATCGTGGTGAACGT
AACCTGCTGTATATTGTTGTTGTTGATGGTAAAGGCAACATCGTGGAACAGTATAGTCTGAACGAAATTATCAACA
ACTTTAACGGCATCCGCATCAAAACCGACTATCATAGCCTGCTGGACAAGAAAGAAAAAGAACGTTTTGAAGCACG
TCAGAACTGGACCAGTATTGAAAACATCAAAGAACTGAAAGCCGGTTATATTAGCCAGGTGGTTCATAAAATCTGT
GAGCTGGTAGAAAAATACGATGCAGTTATTGCACTGGAAGATCTGAATAGCGGTTTCAAAAATAGCCGTGTGAAAG
TCGAAAAACAGGTGTATCAGAAATTCGAGAAAATGCTGATCGACAAACTGAACTACATGGTCGACAAAAAAAGCAA
TCCGTGTGCAACCGGTGGTGCACTGAAAGGTTATCAGATTACCAACAAATTTGAAAGCTTTAAAAGCATGAGCACC
CAGAACGGCTTTATCTTCTATATTCCGGCATGGCTGACCAGCAAAATTGATCCGAGCACCGGTTTTGTGAACCTGC
TGAAAACAAAATATACCTCCATTGCCGACAGCAAGAAGTTTATTAGCAGCTTTGATCGCATTATGTATGTTCCGGA
AGAGGACCTGTTTGAATTCGCACTGGATTACAAAAATTTCAGCCGTACCGATGCCGACTACATCAAAAAATGGAAA
CTGTACAGCTATGGTAACCGCATTCGCATTTTTCGCAACCCGAAGAAAAACAATGTGTTCGATTGGGAAGAAGTTT
GTCTGACCAGCGCATATAAAGAACTTTTCAACAAATACGGCATCAACTATCAGCAGGGTGATATTCGTGCACTGCT
GTGTGAACAGAGCGATAAAGCGTTTTATAGCAGTTTTATGGCACTGATGAGCCTGATGCTGCAGATGCGTAATAGC
ATTACCGGTCGCACCGATGTGGATTTTCTGATTAGTCCGGTGAAAAATTCCGATGGCATCTTTTATGATAGCCGCA
ATTACGAAGCACAAGAAAATGCAATTCTGCCGAAAAACGCAGATGCAAATGGTGCATATAACATTGCACGTAAAGT
TCTGTGGGCAATTGGCCAGTTTAAGAAAGCAGAAGATGAGAAGCTGGACAAAGTGAAAATTGCGATCAGCAATAAA
GAGTGGCTGGAATACGCACAGACCAGCGTTAAACAT
Mutant E795L LbCas12a DNA sequence
SEQ ID NO: 12
ATGAGCAAACTGGAAAAGTTCACCAACTGTTATAGCCTGAGCAAAACCCTGCGTTTTAAAGCAATTCCGGTTGGTA
AAACCCAAGAGAACATTGATAATAAACGCCTGCTGGTCGAAGATGAAAAACGCGCTGAAGATTATAAAGGCGTGAA
AAAACTGCTGGATCGCTATTATCTGAGCTTCATTAACGATGTGCTGCACAGCATTAAACTGAAGAACCTGAACAAC
TATATCAGCCTGTTTCGTAAAAAAACCCGCACCGAAAAAGAAAACAAAGAGCTGGAAAACCTGGAAATCAATCTGC
GTAAAGAAATCGCCAAAGCGTTTAAAGGTAACGAGGGTTATAAAAGCCTGTTCAAGAAAGACATCATCGAAACCAT
TCTGCCGGAATTTCTGGATGATAAAGATGAAATTGCCCTGGTGAATAGCTTTAATGGCTTTACCACCGCATTTACC
GGCTTTTTTGATAATCGCGAAAACATGTTCAGCGAAGAAGCAAAAAGCACCAGCATTGCATTTCGCTGCATTAATG
AAAATCTGACCCGCTACATTAGCAACATGGATATCTTTGAAAAAGTGGACGCGATCTTCGATAAACACGAAGTGCA
AGAGATCAAAGAGAAAATCCTGAACAGCGATTATGACGTCGAAGATTTTTTTGAAGGCGAGTTCTTTAACTTCGTT
CTGACCCAAGAAGGTATCGACGTTTATAACGCAATTATTGGTGGTTTTGTTACCGAAAGCGGTGAGAAAATCAAAG
GCCTGAATGAATATATCAACCTGTATAACCAGAAAACCAAACAGAAACTGCCGAAATTCAAACCGCTGTATAAACA
GGTTCTGAGCGATCGTGAAAGCCTGAGCTTTTATGGTGAAGGTTATACCAGTGATGAAGAGGTTCTGGAAGTTTTT
CGTAACACCCTGAATAAAAACAGCGAGATCTTTAGCAGCATCAAAAAGCTTGAGAAACTGTTCAAAAACTTTGATG
AGTATAGCAGCGCAGGCATCTTTGTTAAAAATGGTCCGGCAATTAGCACCATCAGCAAAGATATTTTTGGCGAATG
GAATGTGATCCGCGATAAATGGAATGCCGAATATGATGATATCCACCTGAAAAAAAAGGCCGTGGTGACCGAGAAA
TATGAAGATGATCGTCGTAAAAGCTTCAAGAAAATTGGTAGCTTTAGCCTGGAACAGCTGCAAGAATATGCAGATG
CAGATCTGAGCGTTGTGGAAAAACTGAAAGAAATCATCATTCAGAAGGTGGACGAGATCTATAAAGTTTATGGTAG
CAGCGAAAAACTGTTCGATGCAGATTTTGTTCTGGAAAAAAGCCTGAAAAAGAATGATGCCGTTGTGGCCATTATG
AAAGATCTGCTGGATAGCGTTAAGAGCTTCGAGAATTACATCAAAGCCTTTTTTGGTGAGGGCAAAGAAACCAATC
GTGATGAAAGTTTCTATGGCGATTTTGTGCTGGCCTATGATATTCTGCTGAAAGTGGACCATATTTATGATGCCAT
TCGCAATTATGTTACCCAGAAACCGTATAGCAAAGACAAGTTCAAACTGTACTTTCAGAACCCGCAGTTTATGGGT
GGTTGGGATAAAGATAAAGAAACCGATTATCGTGCCACCATCCTGCGTTATGGTAGTAAATACTATCTGGCCATCA
TGGACAAAAAATACGCAAAATGCCTGCAGAAAATCGACAAAGATGATGTGAATGGCAACTATGAAAAAATCAACTA
CAAACTGCTGCCTGGTCCGAATAAAATGCTGCCGAAAGTGTTCTTTAGCAAGAAATGGATGGCCTATTATAACCCG
AGCGAGGATATTCAAAAGATCTACAAAAATGGCACCTTTAAAAAGGGCGACATGTTCAATCTGAACGATTGCCACA
AACTGATCGATTTCTTCAAAGATTCAATTTCGCGTTATCCGAAATGGTCCAATGCCTATGATTTTAACTTTAGCGA
AACCGAAAAATACAAAGACATTGCCGGTTTTTATCGCGAAGTGGAAGAACAGGGCTATAAAGTGAGCTTTGAAAGC
GCAAGCAAAAAAGAGGTTGATAAGCTGGTTGAAGAGGGCAAACTGTATATGTTCCAGATTTACAACAAAGATTTTA
GCGACAAAAGCCATGGCACCCCGAATCTGCATACCATGTACTTTAAACTGCTGTTCGACGAAAATAACCATGGTCA
GATTCGTCTGAGCGGTGGTGCCGAACTGTTTATGCGTCGTGCAAGTCTGAAAAAAGAAGAACTGGTTGTTCATCCG
GCAAATAGCCCGATTGCAAACAAAAATCCGGACAATCCGAAAAAAACCACGACACTGAGCTATGATGTGTATAAAG
ACAAACGTTTTAGCGAGGATCAGTATCTGCTGCATATCCCGATTGCCATCAATAAATGCCCGAAAAACATCTTTAA
GATCAACACCGAAGTTCGCGTGCTGCTGAAACATGATGATAATCCGTATGTGATTGGCATTGATCGTGGTGAACGT
AACCTGCTGTATATTGTTGTTGTTGATGGTAAAGGCAACATCGTGGAACAGTATAGTCTGAACGAAATTATCAACA
ACTTTAACGGCATCCGCATCAAAACCGACTATCATAGCCTGCTGGACAAGAAAGAAAAAGAACGTTTTGAAGCACG
TCAGAACTGGACCAGTATTGAAAACATCAAAGAACTGAAAGCCGGTTATATTAGCCAGGTGGTTCATAAAATCTGT
GAGCTGGTAGAAAAATACGATGCAGTTATTGCACTGGAAGATCTGAATAGCGGTTTCAAAAATAGCCGTGTGAAAG
TCGAAAAACAGGTGTATCAGAAATTCGAGAAAATGCTGATCGACAAACTGAACTACATGGTCGACAAAAAAAGCAA
TCCGTGTGCAACCGGTGGTGCACTGAAAGGTTATCAGATTACCAACAAATTTGAAAGCTTTAAAAGCATGAGCACC
CAGAACGGCTTTATCTTCTATATTCCGGCATGGCTGACCAGCAAAATTGATCCGAGCACCGGTTTTGTGAACCTGC
TGAAAACAAAATATACCTCCATTGCCGACAGCAAGAAGTTTATTAGCAGCTTTGATCGCATTATGTATGTTCCGGA
AGAGGACCTGTTTGAATTCGCACTGGATTACAAAAATTTCAGCCGTACCGATGCCGACTACATCAAAAAATGGAAA
CTGTACAGCTATGGTAACCGCATTCGCATTTTTCGCAACCCGAAGAAAAACAATGTGTTCGATTGGGAAGAAGTTT
GTCTGACCAGCGCATATAAAGAACTTTTCAACAAATACGGCATCAACTATCAGCAGGGTGATATTCGTGCACTGCT
GTGTGAACAGAGCGATAAAGCGTTTTATAGCAGTTTTATGGCACTGATGAGCCTGATGCTGCAGATGCGTAATAGC
ATTACCGGTCGCACCGATGTGGATTTTCTGATTAGTCCGGTGAAAAATTCCGATGGCATCTTTTATGATAGCCGCA
ATTACGAAGCACAAGAAAATGCAATTCTGCCGAAAAACGCAGATGCAAATGGTGCATATAACATTGCACGTAAAGT
TCTGTGGGCAATTGGCCAGTTTAAGAAAGCAGAAGATGAGAAGCTGGACAAAGTGAAAATTGCGATCAGCAATAAA
GAGTGGCTGGAATACGCACAGACCAGCGTTAAACAT
Mutant N527R/D559P LbCas12a DNA sequence
SEQ ID NO: 13
ATGAGCAAACTGGAAAAGTTCACCAACTGTTATAGCCTGAGCAAAACCCTGCGTTTTAAAGCAATTCCGGTTGGTA
AAACCCAAGAGAACATTGATAATAAACGCCTGCTGGTCGAAGATGAAAAACGCGCTGAAGATTATAAAGGCGTGAA
AAAACTGCTGGATCGCTATTATCTGAGCTTCATTAACGATGTGCTGCACAGCATTAAACTGAAGAACCTGAACAAC
TATATCAGCCTGTTTCGTAAAAAAACCCGCACCGAAAAAGAAAACAAAGAGCTGGAAAACCTGGAAATCAATCTGC
GTAAAGAAATCGCCAAAGCGTTTAAAGGTAACGAGGGTTATAAAAGCCTGTTCAAGAAAGACATCATCGAAACCAT
TCTGCCGGAATTTCTGGATGATAAAGATGAAATTGCCCTGGTGAATAGCTTTAATGGCTTTACCACCGCATTTACC
GGCTTTTTTGATAATCGCGAAAACATGTTCAGCGAAGAAGCAAAAAGCACCAGCATTGCATTTCGCTGCATTAATG
AAAATCTGACCCGCTACATTAGCAACATGGATATCTTTGAAAAAGTGGACGCGATCTTCGATAAACACGAAGTGCA
AGAGATCAAAGAGAAAATCCTGAACAGCGATTATGACGTCGAAGATTTTTTTGAAGGCGAGTTCTTTAACTTCGTT
CTGACCCAAGAAGGTATCGACGTTTATAACGCAATTATTGGTGGTTTTGTTACCGAAAGCGGTGAGAAAATCAAAG
GCCTGAATGAATATATCAACCTGTATAACCAGAAAACCAAACAGAAACTGCCGAAATTCAAACCGCTGTATAAACA
GGTTCTGAGCGATCGTGAAAGCCTGAGCTTTTATGGTGAAGGTTATACCAGTGATGAAGAGGTTCTGGAAGTTTTT
CGTAACACCCTGAATAAAAACAGCGAGATCTTTAGCAGCATCAAAAAGCTTGAGAAACTGTTCAAAAACTTTGATG
AGTATAGCAGCGCAGGCATCTTTGTTAAAAATGGTCCGGCAATTAGCACCATCAGCAAAGATATTTTTGGCGAATG
GAATGTGATCCGCGATAAATGGAATGCCGAATATGATGATATCCACCTGAAAAAAAAGGCCGTGGTGACCGAGAAA
TATGAAGATGATCGTCGTAAAAGCTTCAAGAAAATTGGTAGCTTTAGCCTGGAACAGCTGCAAGAATATGCAGATG
CAGATCTGAGCGTTGTGGAAAAACTGAAAGAAATCATCATTCAGAAGGTGGACGAGATCTATAAAGTTTATGGTAG
CAGCGAAAAACTGTTCGATGCAGATTTTGTTCTGGAAAAAAGCCTGAAAAAGAATGATGCCGTTGTGGCCATTATG
AAAGATCTGCTGGATAGCGTTAAGAGCTTCGAGAATTACATCAAAGCCTTTTTTGGTGAGGGCAAAGAAACCAATC
GTGATGAAAGTTTCTATGGCGATTTTGTGCTGGCCTATGATATTCTGCTGAAAGTGGACCATATTTATGATGCCAT
TCGCAATTATGTTACCCAGAAACCGTATAGCAAAGACAAGTTCAAACTGTACTTTCAGCGTCCGCAGTTTATGGGT
GGTTGGGATAAAGATAAAGAAACCGATTATCGTGCCACCATCCTGCGTTATGGTAGTAAATACTATCTGGCCATCA
TGCCGAAAAAATACGCAAAATGCCTGCAGAAAATCGACAAAGATGATGTGAATGGCAACTATGAAAAAATCAACTA
CAAACTGCTGCCTGGTCCGAATAAAATGCTGCCGAAAGTGTTCTTTAGCAAGAAATGGATGGCCTATTATAACCCG
AGCGAGGATATTCAAAAGATCTACAAAAATGGCACCTTTAAAAAGGGCGACATGTTCAATCTGAACGATTGCCACA
AACTGATCGATTTCTTCAAAGATTCAATTTCGCGTTATCCGAAATGGTCCAATGCCTATGATTTTAACTTTAGCGA
AACCGAAAAATACAAAGACATTGCCGGTTTTTATCGCGAAGTGGAAGAACAGGGCTATAAAGTGAGCTTTGAAAGC
GCAAGCAAAAAAGAGGTTGATAAGCTGGTTGAAGAGGGCAAACTGTATATGTTCCAGATTTACAACAAAGATTTTA
GCGACAAAAGCCATGGCACCCCGAATCTGCATACCATGTACTTTAAACTGCTGTTCGACGAAAATAACCATGGTCA
GATTCGTCTGAGCGGTGGTGCCGAACTGTTTATGCGTCGTGCAAGTCTGAAAAAAGAAGAACTGGTTGTTCATCCG
GCAAATAGCCCGATTGCAAACAAAAATCCGGACAATCCGAAAAAAACCACGACACTGAGCTATGATGTGTATAAAG
ACAAACGTTTTAGCGAGGATCAGTATGAACTGCATATCCCGATTGCCATCAATAAATGCCCGAAAAACATCTTTAA
GATCAACACCGAAGTTCGCGTGCTGCTGAAACATGATGATAATCCGTATGTGATTGGCATTGATCGTGGTGAACGT
AACCTGCTGTATATTGTTGTTGTTGATGGTAAAGGCAACATCGTGGAACAGTATAGTCTGAACGAAATTATCAACA
ACTTTAACGGCATCCGCATCAAAACCGACTATCATAGCCTGCTGGACAAGAAAGAAAAAGAACGTTTTGAAGCACG
TCAGAACTGGACCAGTATTGAAAACATCAAAGAACTGAAAGCCGGTTATATTAGCCAGGTGGTTCATAAAATCTGT
GAGCTGGTAGAAAAATACGATGCAGTTATTGCACTGGAAGATCTGAATAGCGGTTTCAAAAATAGCCGTGTGAAAG
TCGAAAAACAGGTGTATCAGAAATTCGAGAAAATGCTGATCGACAAACTGAACTACATGGTCGACAAAAAAAGCAA
TCCGTGTGCAACCGGTGGTGCACTGAAAGGTTATCAGATTACCAACAAATTTGAAAGCTTTAAAAGCATGAGCACC
CAGAACGGCTTTATCTTCTATATTCCGGCATGGCTGACCAGCAAAATTGATCCGAGCACCGGTTTTGTGAACCTGC
TGAAAACAAAATATACCTCCATTGCCGACAGCAAGAAGTTTATTAGCAGCTTTGATCGCATTATGTATGTTCCGGA
AGAGGACCTGTTTGAATTCGCACTGGATTACAAAAATTTCAGCCGTACCGATGCCGACTACATCAAAAAATGGAAA
CTGTACAGCTATGGTAACCGCATTCGCATTTTTCGCAACCCGAAGAAAAACAATGTGTTCGATTGGGAAGAAGTTT
GTCTGACCAGCGCATATAAAGAACTTTTCAACAAATACGGCATCAACTATCAGCAGGGTGATATTCGTGCACTGCT
GTGTGAACAGAGCGATAAAGCGTTTTATAGCAGTTTTATGGCACTGATGAGCCTGATGCTGCAGATGCGTAATAGC
ATTACCGGTCGCACCGATGTGGATTTTCTGATTAGTCCGGTGAAAAATTCCGATGGCATCTTTTATGATAGCCGCA
ATTACGAAGCACAAGAAAATGCAATTCTGCCGAAAAACGCAGATGCAAATGGTGCATATAACATTGCACGTAAAGT
TCTGTGGGCAATTGGCCAGTTTAAGAAAGCAGAAGATGAGAAGCTGGACAAAGTGAAAATTGCGATCAGCAATAAA
GAGTGGCTGGAATACGCACAGACCAGCGTTAAACAT
Mutant N527R/E795L LbCas12a DNA sequence
SEQ ID NO: 14
ATGAGCAAACTGGAAAAGTTCACCAACTGTTATAGCCTGAGCAAAACCCTGCGTTTTAAAGCAATTCCGGTTGGTA
AAACCCAAGAGAACATTGATAATAAACGCCTGCTGGTCGAAGATGAAAAACGCGCTGAAGATTATAAAGGCGTGAA
AAAACTGCTGGATCGCTATTATCTGAGCTTCATTAACGATGTGCTGCACAGCATTAAACTGAAGAACCTGAACAAC
TATATCAGCCTGTTTCGTAAAAAAACCCGCACCGAAAAAGAAAACAAAGAGCTGGAAAACCTGGAAATCAATCTGC
GTAAAGAAATCGCCAAAGCGTTTAAAGGTAACGAGGGTTATAAAAGCCTGTTCAAGAAAGACATCATCGAAACCAT
TCTGCCGGAATTTCTGGATGATAAAGATGAAATTGCCCTGGTGAATAGCTTTAATGGCTTTACCACCGCATTTACC
GGCTTTTTTGATAATCGCGAAAACATGTTCAGCGAAGAAGCAAAAAGCACCAGCATTGCATTTCGCTGCATTAATG
AAAATCTGACCCGCTACATTAGCAACATGGATATCTTTGAAAAAGTGGACGCGATCTTCGATAAACACGAAGTGCA
AGAGATCAAAGAGAAAATCCTGAACAGCGATTATGACGTCGAAGATTTTTTTGAAGGCGAGTTCTTTAACTTCGTT
CTGACCCAAGAAGGTATCGACGTTTATAACGCAATTATTGGTGGTTTTGTTACCGAAAGCGGTGAGAAAATCAAAG
GCCTGAATGAATATATCAACCTGTATAACCAGAAAACCAAACAGAAACTGCCGAAATTCAAACCGCTGTATAAACA
GGTTCTGAGCGATCGTGAAAGCCTGAGCTTTTATGGTGAAGGTTATACCAGTGATGAAGAGGTTCTGGAAGTTTTT
CGTAACACCCTGAATAAAAACAGCGAGATCTTTAGCAGCATCAAAAAGCTTGAGAAACTGTTCAAAAACTTTGATG
AGTATAGCAGCGCAGGCATCTTTGTTAAAAATGGTCCGGCAATTAGCACCATCAGCAAAGATATTTTTGGCGAATG
GAATGTGATCCGCGATAAATGGAATGCCGAATATGATGATATCCACCTGAAAAAAAAGGCCGTGGTGACCGAGAAA
TATGAAGATGATCGTCGTAAAAGCTTCAAGAAAATTGGTAGCTTTAGCCTGGAACAGCTGCAAGAATATGCAGATG
CAGATCTGAGCGTTGTGGAAAAACTGAAAGAAATCATCATTCAGAAGGTGGACGAGATCTATAAAGTTTATGGTAG
CAGCGAAAAACTGTTCGATGCAGATTTTGTTCTGGAAAAAAGCCTGAAAAAGAATGATGCCGTTGTGGCCATTATG
AAAGATCTGCTGGATAGCGTTAAGAGCTTCGAGAATTACATCAAAGCCTTTTTTGGTGAGGGCAAAGAAACCAATC
GTGATGAAAGTTTCTATGGCGATTTTGTGCTGGCCTATGATATTCTGCTGAAAGTGGACCATATTTATGATGCCAT
TCGCAATTATGTTACCCAGAAACCGTATAGCAAAGACAAGTTCAAACTGTACTTTCAGCGTCCGCAGTTTATGGGT
GGTTGGGATAAAGATAAAGAAACCGATTATCGTGCCACCATCCTGCGTTATGGTAGTAAATACTATCTGGCCATCA
TGGACAAAAAATACGCAAAATGCCTGCAGAAAATCGACAAAGATGATGTGAATGGCAACTATGAAAAAATCAACTA
CAAACTGCTGCCTGGTCCGAATAAAATGCTGCCGAAAGTGTTCTTTAGCAAGAAATGGATGGCCTATTATAACCCG
AGCGAGGATATTCAAAAGATCTACAAAAATGGCACCTTTAAAAAGGGCGACATGTTCAATCTGAACGATTGCCACA
AACTGATCGATTTCTTCAAAGATTCAATTTCGCGTTATCCGAAATGGTCCAATGCCTATGATTTTAACTTTAGCGA
AACCGAAAAATACAAAGACATTGCCGGTTTTTATCGCGAAGTGGAAGAACAGGGCTATAAAGTGAGCTTTGAAAGC
GCAAGCAAAAAAGAGGTTGATAAGCTGGTTGAAGAGGGCAAACTGTATATGTTCCAGATTTACAACAAAGATTTTA
GCGACAAAAGCCATGGCACCCCGAATCTGCATACCATGTACTTTAAACTGCTGTTCGACGAAAATAACCATGGTCA
GATTCGTCTGAGCGGTGGTGCCGAACTGTTTATGCGTCGTGCAAGTCTGAAAAAAGAAGAACTGGTTGTTCATCCG
GCAAATAGCCCGATTGCAAACAAAAATCCGGACAATCCGAAAAAAACCACGACACTGAGCTATGATGTGTATAAAG
ACAAACGTTTTAGCGAGGATCAGTATCTGCTGCATATCCCGATTGCCATCAATAAATGCCCGAAAAACATCTTTAA
GATCAACACCGAAGTTCGCGTGCTGCTGAAACATGATGATAATCCGTATGTGATTGGCATTGATCGTGGTGAACGT
AACCTGCTGTATATTGTTGTTGTTGATGGTAAAGGCAACATCGTGGAACAGTATAGTCTGAACGAAATTATCAACA
ACTTTAACGGCATCCGCATCAAAACCGACTATCATAGCCTGCTGGACAAGAAAGAAAAAGAACGTTTTGAAGCACG
TCAGAACTGGACCAGTATTGAAAACATCAAAGAACTGAAAGCCGGTTATATTAGCCAGGTGGTTCATAAAATCTGT
GAGCTGGTAGAAAAATACGATGCAGTTATTGCACTGGAAGATCTGAATAGCGGTTTCAAAAATAGCCGTGTGAAAG
TCGAAAAACAGGTGTATCAGAAATTCGAGAAAATGCTGATCGACAAACTGAACTACATGGTCGACAAAAAAAGCAA
TCCGTGTGCAACCGGTGGTGCACTGAAAGGTTATCAGATTACCAACAAATTTGAAAGCTTTAAAAGCATGAGCACC
CAGAACGGCTTTATCTTCTATATTCCGGCATGGCTGACCAGCAAAATTGATCCGAGCACCGGTTTTGTGAACCTGC
TGAAAACAAAATATACCTCCATTGCCGACAGCAAGAAGTTTATTAGCAGCTTTGATCGCATTATGTATGTTCCGGA
AGAGGACCTGTTTGAATTCGCACTGGATTACAAAAATTTCAGCCGTACCGATGCCGACTACATCAAAAAATGGAAA
CTGTACAGCTATGGTAACCGCATTCGCATTTTTCGCAACCCGAAGAAAAACAATGTGTTCGATTGGGAAGAAGTTT
GTCTGACCAGCGCATATAAAGAACTTTTCAACAAATACGGCATCAACTATCAGCAGGGTGATATTCGTGCACTGCT
GTGTGAACAGAGCGATAAAGCGTTTTATAGCAGTTTTATGGCACTGATGAGCCTGATGCTGCAGATGCGTAATAGC
ATTACCGGTCGCACCGATGTGGATTTTCTGATTAGTCCGGTGAAAAATTCCGATGGCATCTTTTATGATAGCCGCA
ATTACGAAGCACAAGAAAATGCAATTCTGCCGAAAAACGCAGATGCAAATGGTGCATATAACATTGCACGTAAAGT
TCTGTGGGCAATTGGCCAGTTTAAGAAAGCAGAAGATGAGAAGCTGGACAAAGTGAAAATTGCGATCAGCAATAAA
GAGTGGCTGGAATACGCACAGACCAGCGTTAAACAT
Mutant D559P/E795L LbCas12a DNA sequence
SEQ ID NO: 15
ATGAGCAAACTGGAAAAGTTCACCAACTGTTATAGCCTGAGCAAAACCCTGCGTTTTAAAGCAATTCCGGTTGGTA
AAACCCAAGAGAACATTGATAATAAACGCCTGCTGGTCGAAGATGAAAAACGCGCTGAAGATTATAAAGGCGTGAA
AAAACTGCTGGATCGCTATTATCTGAGCTTCATTAACGATGTGCTGCACAGCATTAAACTGAAGAACCTGAACAAC
TATATCAGCCTGTTTCGTAAAAAAACCCGCACCGAAAAAGAAAACAAAGAGCTGGAAAACCTGGAAATCAATCTGC
GTAAAGAAATCGCCAAAGCGTTTAAAGGTAACGAGGGTTATAAAAGCCTGTTCAAGAAAGACATCATCGAAACCAT
TCTGCCGGAATTTCTGGATGATAAAGATGAAATTGCCCTGGTGAATAGCTTTAATGGCTTTACCACCGCATTTACC
GGCTTTTTTGATAATCGCGAAAACATGTTCAGCGAAGAAGCAAAAAGCACCAGCATTGCATTTCGCTGCATTAATG
AAAATCTGACCCGCTACATTAGCAACATGGATATCTTTGAAAAAGTGGACGCGATCTTCGATAAACACGAAGTGCA
AGAGATCAAAGAGAAAATCCTGAACAGCGATTATGACGTCGAAGATTTTTTTGAAGGCGAGTTCTTTAACTTCGTT
CTGACCCAAGAAGGTATCGACGTTTATAACGCAATTATTGGTGGTTTTGTTACCGAAAGCGGTGAGAAAATCAAAG
GCCTGAATGAATATATCAACCTGTATAACCAGAAAACCAAACAGAAACTGCCGAAATTCAAACCGCTGTATAAACA
GGTTCTGAGCGATCGTGAAAGCCTGAGCTTTTATGGTGAAGGTTATACCAGTGATGAAGAGGTTCTGGAAGTTTTT
CGTAACACCCTGAATAAAAACAGCGAGATCTTTAGCAGCATCAAAAAGCTTGAGAAACTGTTCAAAAACTTTGATG
AGTATAGCAGCGCAGGCATCTTTGTTAAAAATGGTCCGGCAATTAGCACCATCAGCAAAGATATTTTTGGCGAATG
GAATGTGATCCGCGATAAATGGAATGCCGAATATGATGATATCCACCTGAAAAAAAAGGCCGTGGTGACCGAGAAA
TATGAAGATGATCGTCGTAAAAGCTTCAAGAAAATTGGTAGCTTTAGCCTGGAACAGCTGCAAGAATATGCAGATG
CAGATCTGAGCGTTGTGGAAAAACTGAAAGAAATCATCATTCAGAAGGTGGACGAGATCTATAAAGTTTATGGTAG
CAGCGAAAAACTGTTCGATGCAGATTTTGTTCTGGAAAAAAGCCTGAAAAAGAATGATGCCGTTGTGGCCATTATG
AAAGATCTGCTGGATAGCGTTAAGAGCTTCGAGAATTACATCAAAGCCTTTTTTGGTGAGGGCAAAGAAACCAATC
GTGATGAAAGTTTCTATGGCGATTTTGTGCTGGCCTATGATATTCTGCTGAAAGTGGACCATATTTATGATGCCAT
TCGCAATTATGTTACCCAGAAACCGTATAGCAAAGACAAGTTCAAACTGTACTTTCAGAACCCGCAGTTTATGGGT
GGTTGGGATAAAGATAAAGAAACCGATTATCGTGCCACCATCCTGCGTTATGGTAGTAAATACTATCTGGCCATCA
TGCCGAAAAAATACGCAAAATGCCTGCAGAAAATCGACAAAGATGATGTGAATGGCAACTATGAAAAAATCAACTA
CAAACTGCTGCCTGGTCCGAATAAAATGCTGCCGAAAGTGTTCTTTAGCAAGAAATGGATGGCCTATTATAACCCG
AGCGAGGATATTCAAAAGATCTACAAAAATGGCACCTTTAAAAAGGGCGACATGTTCAATCTGAACGATTGCCACA
AACTGATCGATTTCTTCAAAGATTCAATTTCGCGTTATCCGAAATGGTCCAATGCCTATGATTTTAACTTTAGCGA
AACCGAAAAATACAAAGACATTGCCGGTTTTTATCGCGAAGTGGAAGAACAGGGCTATAAAGTGAGCTTTGAAAGC
GCAAGCAAAAAAGAGGTTGATAAGCTGGTTGAAGAGGGCAAACTGTATATGTTCCAGATTTACAACAAAGATTTTA
GCGACAAAAGCCATGGCACCCCGAATCTGCATACCATGTACTTTAAACTGCTGTTCGACGAAAATAACCATGGTCA
GATTCGTCTGAGCGGTGGTGCCGAACTGTTTATGCGTCGTGCAAGTCTGAAAAAAGAAGAACTGGTTGTTCATCCG
GCAAATAGCCCGATTGCAAACAAAAATCCGGACAATCCGAAAAAAACCACGACACTGAGCTATGATGTGTATAAAG
ACAAACGTTTTAGCGAGGATCAGTATCTGCTGCATATCCCGATTGCCATCAATAAATGCCCGAAAAACATCTTTAA
GATCAACACCGAAGTTCGCGTGCTGCTGAAACATGATGATAATCCGTATGTGATTGGCATTGATCGTGGTGAACGT
AACCTGCTGTATATTGTTGTTGTTGATGGTAAAGGCAACATCGTGGAACAGTATAGTCTGAACGAAATTATCAACA
ACTTTAACGGCATCCGCATCAAAACCGACTATCATAGCCTGCTGGACAAGAAAGAAAAAGAACGTTTTGAAGCACG
TCAGAACTGGACCAGTATTGAAAACATCAAAGAACTGAAAGCCGGTTATATTAGCCAGGTGGTTCATAAAATCTGT
GAGCTGGTAGAAAAATACGATGCAGTTATTGCACTGGAAGATCTGAATAGCGGTTTCAAAAATAGCCGTGTGAAAG
TCGAAAAACAGGTGTATCAGAAATTCGAGAAAATGCTGATCGACAAACTGAACTACATGGTCGACAAAAAAAGCAA
TCCGTGTGCAACCGGTGGTGCACTGAAAGGTTATCAGATTACCAACAAATTTGAAAGCTTTAAAAGCATGAGCACC
CAGAACGGCTTTATCTTCTATATTCCGGCATGGCTGACCAGCAAAATTGATCCGAGCACCGGTTTTGTGAACCTGC
TGAAAACAAAATATACCTCCATTGCCGACAGCAAGAAGTTTATTAGCAGCTTTGATCGCATTATGTATGTTCCGGA
AGAGGACCTGTTTGAATTCGCACTGGATTACAAAAATTTCAGCCGTACCGATGCCGACTACATCAAAAAATGGAAA
CTGTACAGCTATGGTAACCGCATTCGCATTTTTCGCAACCCGAAGAAAAACAATGTGTTCGATTGGGAAGAAGTTT
GTCTGACCAGCGCATATAAAGAACTTTTCAACAAATACGGCATCAACTATCAGCAGGGTGATATTCGTGCACTGCT
GTGTGAACAGAGCGATAAAGCGTTTTATAGCAGTTTTATGGCACTGATGAGCCTGATGCTGCAGATGCGTAATAGC
ATTACCGGTCGCACCGATGTGGATTTTCTGATTAGTCCGGTGAAAAATTCCGATGGCATCTTTTATGATAGCCGCA
ATTACGAAGCACAAGAAAATGCAATTCTGCCGAAAAACGCAGATGCAAATGGTGCATATAACATTGCACGTAAAGT
TCTGTGGGCAATTGGCCAGTTTAAGAAAGCAGAAGATGAGAAGCTGGACAAAGTGAAAATTGCGATCAGCAATAAA
GAGTGGCTGGAATACGCACAGACCAGCGTTAAACAT
Mutant N527R/D559P/E795L LbCas12a DNA sequence
SEQ ID NO: 16
ATGAGCAAACTGGAAAAGTTCACCAACTGTTATAGCCTGAGCAAAACCCTGCGTTTTAAAGCAATTCCGGTTGGTA
AAACCCAAGAGAACATTGATAATAAACGCCTGCTGGTCGAAGATGAAAAACGCGCTGAAGATTATAAAGGCGTGAA
AAAACTGCTGGATCGCTATTATCTGAGCTTCATTAACGATGTGCTGCACAGCATTAAACTGAAGAACCTGAACAAC
TATATCAGCCTGTTTCGTAAAAAAACCCGCACCGAAAAAGAAAACAAAGAGCTGGAAAACCTGGAAATCAATCTGC
GTAAAGAAATCGCCAAAGCGTTTAAAGGTAACGAGGGTTATAAAAGCCTGTTCAAGAAAGACATCATCGAAACCAT
TCTGCCGGAATTTCTGGATGATAAAGATGAAATTGCCCTGGTGAATAGCTTTAATGGCTTTACCACCGCATTTACC
GGCTTTTTTGATAATCGCGAAAACATGTTCAGCGAAGAAGCAAAAAGCACCAGCATTGCATTTCGCTGCATTAATG
AAAATCTGACCCGCTACATTAGCAACATGGATATCTTTGAAAAAGTGGACGCGATCTTCGATAAACACGAAGTGCA
AGAGATCAAAGAGAAAATCCTGAACAGCGATTATGACGTCGAAGATTTTTTTGAAGGCGAGTTCTTTAACTTCGTT
CTGACCCAAGAAGGTATCGACGTTTATAACGCAATTATTGGTGGTTTTGTTACCGAAAGCGGTGAGAAAATCAAAG
GCCTGAATGAATATATCAACCTGTATAACCAGAAAACCAAACAGAAACTGCCGAAATTCAAACCGCTGTATAAACA
GGTTCTGAGCGATCGTGAAAGCCTGAGCTTTTATGGTGAAGGTTATACCAGTGATGAAGAGGTTCTGGAAGTTTTT
CGTAACACCCTGAATAAAAACAGCGAGATCTTTAGCAGCATCAAAAAGCTTGAGAAACTGTTCAAAAACTTTGATG
AGTATAGCAGCGCAGGCATCTTTGTTAAAAATGGTCCGGCAATTAGCACCATCAGCAAAGATATTTTTGGCGAATG
GAATGTGATCCGCGATAAATGGAATGCCGAATATGATGATATCCACCTGAAAAAAAAGGCCGTGGTGACCGAGAAA
TATGAAGATGATCGTCGTAAAAGCTTCAAGAAAATTGGTAGCTTTAGCCTGGAACAGCTGCAAGAATATGCAGATG
CAGATCTGAGCGTTGTGGAAAAACTGAAAGAAATCATCATTCAGAAGGTGGACGAGATCTATAAAGTTTATGGTAG
CAGCGAAAAACTGTTCGATGCAGATTTTGTTCTGGAAAAAAGCCTGAAAAAGAATGATGCCGTTGTGGCCATTATG
AAAGATCTGCTGGATAGCGTTAAGAGCTTCGAGAATTACATCAAAGCCTTTTTTGGTGAGGGCAAAGAAACCAATC
GTGATGAAAGTTTCTATGGCGATTTTGTGCTGGCCTATGATATTCTGCTGAAAGTGGACCATATTTATGATGCCAT
TCGCAATTATGTTACCCAGAAACCGTATAGCAAAGACAAGTTCAAACTGTACTTTCAGCGTCCGCAGTTTATGGGT
GGTTGGGATAAAGATAAAGAAACCGATTATCGTGCCACCATCCTGCGTTATGGTAGTAAATACTATCTGGCCATCA
TGCCGAAAAAATACGCAAAATGCCTGCAGAAAATCGACAAAGATGATGTGAATGGCAACTATGAAAAAATCAACTA
CAAACTGCTGCCTGGTCCGAATAAAATGCTGCCGAAAGTGTTCTTTAGCAAGAAATGGATGGCCTATTATAACCCG
AGCGAGGATATTCAAAAGATCTACAAAAATGGCACCTTTAAAAAGGGCGACATGTTCAATCTGAACGATTGCCACA
AACTGATCGATTTCTTCAAAGATTCAATTTCGCGTTATCCGAAATGGTCCAATGCCTATGATTTTAACTTTAGCGA
AACCGAAAAATACAAAGACATTGCCGGTTTTTATCGCGAAGTGGAAGAACAGGGCTATAAAGTGAGCTTTGAAAGC
GCAAGCAAAAAAGAGGTTGATAAGCTGGTTGAAGAGGGCAAACTGTATATGTTCCAGATTTACAACAAAGATTTTA
GCGACAAAAGCCATGGCACCCCGAATCTGCATACCATGTACTTTAAACTGCTGTTCGACGAAAATAACCATGGTCA
GATTCGTCTGAGCGGTGGTGCCGAACTGTTTATGCGTCGTGCAAGTCTGAAAAAAGAAGAACTGGTTGTTCATCCG
GCAAATAGCCCGATTGCAAACAAAAATCCGGACAATCCGAAAAAAACCACGACACTGAGCTATGATGTGTATAAAG
ACAAACGTTTTAGCGAGGATCAGTATCTGCTGCATATCCCGATTGCCATCAATAAATGCCCGAAAAACATCTTTAA
GATCAACACCGAAGTTCGCGTGCTGCTGAAACATGATGATAATCCGTATGTGATTGGCATTGATCGTGGTGAACGT
AACCTGCTGTATATTGTTGTTGTTGATGGTAAAGGCAACATCGTGGAACAGTATAGTCTGAACGAAATTATCAACA
ACTTTAACGGCATCCGCATCAAAACCGACTATCATAGCCTGCTGGACAAGAAAGAAAAAGAACGTTTTGAAGCACG
TCAGAACTGGACCAGTATTGAAAACATCAAAGAACTGAAAGCCGGTTATATTAGCCAGGTGGTTCATAAAATCTGT
GAGCTGGTAGAAAAATACGATGCAGTTATTGCACTGGAAGATCTGAATAGCGGTTTCAAAAATAGCCGTGTGAAAG
TCGAAAAACAGGTGTATCAGAAATTCGAGAAAATGCTGATCGACAAACTGAACTACATGGTCGACAAAAAAAGCAA
TCCGTGTGCAACCGGTGGTGCACTGAAAGGTTATCAGATTACCAACAAATTTGAAAGCTTTAAAAGCATGAGCACC
CAGAACGGCTTTATCTTCTATATTCCGGCATGGCTGACCAGCAAAATTGATCCGAGCACCGGTTTTGTGAACCTGC
TGAAAACAAAATATACCTCCATTGCCGACAGCAAGAAGTTTATTAGCAGCTTTGATCGCATTATGTATGTTCCGGA
AGAGGACCTGTTTGAATTCGCACTGGATTACAAAAATTTCAGCCGTACCGATGCCGACTACATCAAAAAATGGAAA
CTGTACAGCTATGGTAACCGCATTCGCATTTTTCGCAACCCGAAGAAAAACAATGTGTTCGATTGGGAAGAAGTTT
GTCTGACCAGCGCATATAAAGAACTTTTCAACAAATACGGCATCAACTATCAGCAGGGTGATATTCGTGCACTGCT
GTGTGAACAGAGCGATAAAGCGTTTTATAGCAGTTTTATGGCACTGATGAGCCTGATGCTGCAGATGCGTAATAGC
ATTACCGGTCGCACCGATGTGGATTTTCTGATTAGTCCGGTGAAAAATTCCGATGGCATCTTTTATGATAGCCGCA
ATTACGAAGCACAAGAAAATGCAATTCTGCCGAAAAACGCAGATGCAAATGGTGCATATAACATTGCACGTAAAGT
TCTGTGGGCAATTGGCCAGTTTAAGAAAGCAGAAGATGAGAAGCTGGACAAAGTGAAAATTGCGATCAGCAATAAA
GAGTGGCTGGAATACGCACAGACCAGCGTTAAACAT
WT AsCas12a DNA sequence
SEQ ID NO: 17
ATGACCCAGTTTGAAGGTTTCACCAATCTGTATCAGGTTAGCAAAACCCTGCGTTTTGAACTGATTCCGCAGGGTA
AAACCCTGAAACATATTCAAGAACAGGGCTTCATCGAAGAGGATAAAGCACGTAACGATCACTACAAAGAACTGAA
ACCGATTATCGACCGCATCTATAAAACCTATGCAGATCAGTGTCTGCAGCTGGTTCAGCTGGATTGGGAAAATCTG
AGCGCAGCAATTGATAGTTATCGCAAAGAAAAAACCGAAGAAACCCGTAATGCACTGATTGAAGAACAGGCAACCT
ATCGTAATGCCATCCATGATTATTTCATTGGTCGTACCGATAATCTGACCGATGCAATTAACAAACGTCACGCCGA
AATCTATAAAGGCCTGTTTAAAGCCGAACTGTTTAATGGCAAAGTTCTGAAACAGCTGGGCACCGTTACCACCACC
GAACATGAAAATGCACTGCTGCGTAGCTTTGATAAATTCACCACCTATTTCAGCGGCTTTTATGAGAATCGCAAAA
ACGTGTTTAGCGCAGAAGATATTAGCACCGCAATTCCGCATCGTATTGTGCAGGATAATTTCCCGAAATTCAAAGA
GAACTGCCACATTTTTACCCGTCTGATTACCGCAGTTCCGAGCCTGCGTGAACATTTTGAAAACGTTAAAAAAGCC
ATCGGCATCTTTGTTAGCACCAGCATTGAAGAAGTTTTTAGCTTCCCGTTTTACAATCAGCTGCTGACCCAGACCC
AGATTGATCTGTATAACCAACTGCTGGGTGGTATTAGCCGTGAAGCAGGCACCGAAAAAATCAAAGGTCTGAATGA
AGTGCTGAATCTGGCCATTCAGAAAAATGATGAAACCGCACATATTATTGCAAGCCTGCCGCATCGTTTTATTCCG
CTGTTCAAACAAATTCTGAGCGATCGTAATACCCTGAGCTTTATTCTGGAAGAATTCAAATCCGATGAAGAGGTGA
TTCAGAGCTTTTGCAAATACAAAACGCTGCTGCGCAATGAAAATGTTCTGGAAACTGCCGAAGCACTGTTTAACGA
ACTGAATAGCATTGATCTGACCCACATCTTTATCAGCCACAAAAAACTGGAAACCATTTCAAGCGCACTGTGTGAT
CATTGGGATACCCTGCGTAATGCCCTGTATGAACGTCGTATTAGCGAACTGACCGGTAAAATTACCAAAAGCGCGA
AAGAAAAAGTTCAGCGCAGTCTGAAACATGAGGATATTAATCTGCAAGAGATTATTAGCGCAGCCGGTAAAGAACT
GTCAGAAGCATTTAAACAGAAAACCAGCGAAATTCTGTCACATGCACATGCAGCACTGGATCAGCCGCTGCCGACC
ACCCTGAAAAAACAAGAAGAAAAAGAAATCCTGAAAAGCCAGCTGGATAGCCTGCTGGGTCTGTATCATCTGCTGG
ACTGGTTTGCAGTTGATGAAAGCAATGAAGTTGATCCGGAATTTAGCGCACGTCTGACCGGCATTAAACTGGAAAT
GGAACCGAGCCTGAGCTTTTATAACAAAGCCCGTAATTATGCCACCAAAAAACCGTATAGCGTCGAAAAATTCAAA
CTGAACTTTCAGATGCCGACCCTGGCAAGCGGTTGGGATGTTAATAAAGAAAAAAACAACGGTGCCATCCTGTTCG
TGAAAAATGGCCTGTATTATCTGGGTATTATGCCGAAACAGAAAGGTCGTTATAAAGCGCTGAGCTTTGAACCGAC
GGAAAAAACCAGTGAAGGTTTTGATAAAATGTACTACGACTATTTTCCGGATGCAGCCAAAATGATTCCGAAATGT
AGCACCCAGCTGAAAGCAGTTACCGCACATTTTCAGACCCATACCACCCCGATTCTGCTGAGCAATAACTTTATTG
AACCGCTGGAAATCACCAAAGAGATCTACGATCTGAATAACCCGGAAAAAGAGCCGAAAAAATTCCAGACCGCATA
TGCAAAAAAAACCGGTGATCAGAAAGGTTATCGTGAAGCGCTGTGTAAATGGATTGATTTCACCCGTGATTTTCTG
AGCAAATACACCAAAACCACCAGTATCGATCTGAGCAGCCTGCGTCCGAGCAGCCAGTATAAAGATCTGGGCGAAT
ATTATGCAGAACTGAATCCGCTGCTGTATCATATTAGCTTTCAGCGTATTGCCGAGAAAGAAATCATGGACGCAGT
TGAAACCGGTAAACTGTACCTGTTCCAGATCTACAATAAAGATTTTGCCAAAGGCCATCATGGCAAACCGAATCTG
CATACCCTGTATTGGACCGGTCTGTTTAGCCCTGAAAATCTGGCAAAAACCTCGATTAAACTGAATGGTCAGGCGG
AACTGTTTTATCGTCCGAAAAGCCGTATGAAACGTATGGCACATCGTCTGGGTGAAAAAATGCTGAACAAAAAACT
GAAAGACCAGAAAACCCCGATCCCGGATACACTGTATCAAGAACTGTATGATTATGTGAACCATCGTCTGAGCCAT
GATCTGAGTGATGAAGCACGTGCCCTGCTGCCGAATGTTATTACCAAAGAAGTTAGCCACGAGATCATTAAAGATC
GTCGTTTTACCAGCGACAAATTCTTTTTTCATGTGCCGATTACCCTGAATTATCAGGCAGCAAATAGCCCGAGCAA
ATTTAACCAGCGTGTTAATGCATATCTGAAAGAACATCCAGAAACGCCGATTATTGGTATTGATCGTGGTGAACGT
AACCTGATTTATATCACCGTTATTGATAGCACCGGCAAAATCCTGGAACAGCGTAGCCTGAATACCATTCAGCAGT
TTGATTACCAGAAAAAACTGGATAATCGCGAGAAAGAACGTGTTGCAGCACGTCAGGCATGGTCAGTTGTTGGTAC
AATTAAAGACCTGAAACAGGGTTATCTGAGCCAGGTTATTCATGAAATTGTGGATCTGATGATTCACTATCAGGCC
GTTGTTGTGCTGGAAAACCTGAATTTTGGCTTTAAAAGCAAACGTACCGGCATTGCAGAAAAAGCAGTTTATCAGC
AGTTCGAGAAAATGCTGATTGACAAACTGAATTGCCTGGTGCTGAAAGATTATCCGGCTGAAAAAGTTGGTGGTGT
TCTGAATCCGTATCAGCTGACCGATCAGTTTACCAGCTTTGCAAAAATGGGCACCCAGAGCGGATTTCTGTTTTAT
GTTCCGGCACCGTATACGAGCAAAATTGATCCGCTGACCGGTTTTGTTGATCCGTTTGTTTGGAAAACCATCAAAA
ACCATGAAAGCCGCAAACATTTTCTGGAAGGTTTCGATTTTCTGCATTACGACGTTAAAACGGGTGATTTCATCCT
GCACTTTAAAATGAATCGCAATCTGAGTTTTCAGCGTGGCCTGCCTGGTTTTATGCCTGCATGGGATATTGTGTTT
GAGAAAAACGAAACACAGTTCGATGCAAAAGGCACCCCGTTTATTGCAGGTAAACGTATTGTTCCGGTGATTGAAA
ATCATCGTTTCACCGGTCGTTATCGCGATCTGTATCCGGCAAATGAACTGATCGCACTGCTGGAAGAGAAAGGTAT
TGTTTTTCGTGATGGCTCAAACATTCTGCCGAAACTGCTGGAAAATGATGATAGCCATGCAATTGATACCATGGTT
GCACTGATTCGTAGCGTTCTGCAGATGCGTAATAGCAATGCAGCAACCGGTGAAGATTACATTAATAGTCCGGTTC
GTGATCTGAATGGTGTTTGTTTTGATAGCCGTTTTCAGAATCCGGAATGGCCGATGGATGCAGATGCAAATGGTGC
ATATCATATTGCACTGAAAGGACAGCTGCTGCTGAACCACCTGAAAGAAAGCAAAGATCTGAAACTGCAAAACGGC
ATTAGCAATCAGGATTGGCTGGCATATATCCAAGAACTGCGTAAC
WT AsCas12a amino acid sequence
SEQ ID NO: 18
MTQFEGFTNLYQVSKTLRFELIPQGKTLKHIQEQGFIEEDKARNDHYKELKPIIDRIYKTYADQCLQLVQLDWENL
SAAIDSYRKEKTEETRNALIEEQATYRNAIHDYFIGRTDNLTDAINKRHAEIYKGLFKAELFNGKVLKQLGTVTTT
EHENALLRSFDKFTTYFSGFYENRKNVFSAEDISTAIPHRIVQDNFPKFKENCHIFTRLITAVPSLREHFENVKKA
IGIFVSTSIEEVFSFPFYNQLLTQTQIDLYNQLLGGISREAGTEKIKGLNEVLNLAIQKNDETAHIIASLPHRFIP
LFKQILSDRNTLSFILEEFKSDEEVIQSFCKYKTLLRNENVLETAEALFNELNSIDLTHIFISHKKLETISSALCD
HWDTLRNALYERRISELTGKITKSAKEKVQRSLKHEDINLQEIISAAGKELSEAFKQKTSEILSHAHAALDQPLPT
TLKKQEEKEILKSQLDSLLGLYHLLDWFAVDESNEVDPEFSARLTGIKLEMEPSLSFYNKARNYATKKPYSVEKFK
LNFQMPTLASGWDVNKEKNNGAILFVKNGLYYLGIMPKQKGRYKALSFEPTEKTSEGFDKMYYDYFPDAAKMIPKC
STQLKAVTAHFQTHTTPILLSNNFIEPLEITKEIYDLNNPEKEPKKFQTAYAKKTGDQKGYREALCKWIDFTRDFL
SKYTKTTSIDLSSLRPSSQYKDLGEYYAELNPLLYHISFQRIAEKEIMDAVETGKLYLFQIYNKDFAKGHHGKPNL
HTLYWTGLFSPENLAKTSIKLNGQAELFYRPKSRMKRMAHRLGEKMLNKKLKDQKTPIPDTLYQELYDYVNHRLSH
DLSDEARALLPNVITKEVSHEIIKDRRFTSDKFFFHVPITLNYQAANSPSKFNQRVNAYLKEHPETPIIGIDRGER
NLIYITVIDSTGKILEQRSLNTIQQFDYQKKLDNREKERVAARQAWSVVGTIKDLKQGYLSQVIHEIVDLMIHYQA
VVVLENLNFGFKSKRTGIAEKAVYQQFEKMLIDKLNCLVLKDYPAEKVGGVLNPYQLTDQFTSFAKMGTQSGFLFY
VPAPYTSKIDPLTGFVDPFVWKTIKNHESRKHFLEGFDFLHYDVKTGDFILHFKMNRNLSFQRGLPGFMPAWDIVF
EKNETQFDAKGTPFIAGKRIVPVIENHRFTGRYRDLYPANELIALLEEKGIVFRDGSNILPKLLENDDSHAIDTMV
ALIRSVLQMRNSNAATGEDYINSPVRDLNGVCFDSRFQNPEWPMDADANGAYHIALKGQLLLNHLKESKDLKLQNG
ISNQDWLAYIQELRN
Mutant M537R/F870L AsCas12a DNA sequence
SEQ ID NO: 19
ATGACCCAGTTTGAAGGTTTCACCAATCTGTATCAGGTTAGCAAAACCCTGCGTTTTGAACTGATTCCGCAGGGTA
AAACCCTGAAACATATTCAAGAACAGGGCTTCATCGAAGAGGATAAAGCACGTAACGATCACTACAAAGAACTGAA
ACCGATTATCGACCGCATCTATAAAACCTATGCAGATCAGTGTCTGCAGCTGGTTCAGCTGGATTGGGAAAATCTG
AGCGCAGCAATTGATAGTTATCGCAAAGAAAAAACCGAAGAAACCCGTAATGCACTGATTGAAGAACAGGCAACCT
ATCGTAATGCCATCCATGATTATTTCATTGGTCGTACCGATAATCTGACCGATGCAATTAACAAACGTCACGCCGA
AATCTATAAAGGCCTGTTTAAAGCCGAACTGTTTAATGGCAAAGTTCTGAAACAGCTGGGCACCGTTACCACCACC
GAACATGAAAATGCACTGCTGCGTAGCTTTGATAAATTCACCACCTATTTCAGCGGCTTTTATGAGAATCGCAAAA
ACGTGTTTAGCGCAGAAGATATTAGCACCGCAATTCCGCATCGTATTGTGCAGGATAATTTCCCGAAATTCAAAGA
GAACTGCCACATTTTTACCCGTCTGATTACCGCAGTTCCGAGCCTGCGTGAACATTTTGAAAACGTTAAAAAAGCC
ATCGGCATCTTTGTTAGCACCAGCATTGAAGAAGTTTTTAGCTTCCCGTTTTACAATCAGCTGCTGACCCAGACCC
AGATTGATCTGTATAACCAACTGCTGGGTGGTATTAGCCGTGAAGCAGGCACCGAAAAAATCAAAGGTCTGAATGA
AGTGCTGAATCTGGCCATTCAGAAAAATGATGAAACCGCACATATTATTGCAAGCCTGCCGCATCGTTTTATTCCG
CTGTTCAAACAAATTCTGAGCGATCGTAATACCCTGAGCTTTATTCTGGAAGAATTCAAATCCGATGAAGAGGTGA
TTCAGAGCTTTTGCAAATACAAAACGCTGCTGCGCAATGAAAATGTTCTGGAAACTGCCGAAGCACTGTTTAACGA
ACTGAATAGCATTGATCTGACCCACATCTTTATCAGCCACAAAAAACTGGAAACCATTTCAAGCGCACTGTGTGAT
CATTGGGATACCCTGCGTAATGCCCTGTATGAACGTCGTATTAGCGAACTGACCGGTAAAATTACCAAAAGCGCGA
AAGAAAAAGTTCAGCGCAGTCTGAAACATGAGGATATTAATCTGCAAGAGATTATTAGCGCAGCCGGTAAAGAACT
GTCAGAAGCATTTAAACAGAAAACCAGCGAAATTCTGTCACATGCACATGCAGCACTGGATCAGCCGCTGCCGACC
ACCCTGAAAAAACAAGAAGAAAAAGAAATCCTGAAAAGCCAGCTGGATAGCCTGCTGGGTCTGTATCATCTGCTGG
ACTGGTTTGCAGTTGATGAAAGCAATGAAGTTGATCCGGAATTTAGCGCACGTCTGACCGGCATTAAACTGGAAAT
GGAACCGAGCCTGAGCTTTTATAACAAAGCCCGTAATTATGCCACCAAAAAACCGTATAGCGTCGAAAAATTCAAA
CTGAACTTTCAGCGTCCGACCCTGGCAAGCGGTTGGGATGTTAATAAAGAAAAAAACAACGGTGCCATCCTGTTCG
TGAAAAATGGCCTGTATTATCTGGGTATTATGCCGAAACAGAAAGGTCGTTATAAAGCGCTGAGCTTTGAACCGAC
GGAAAAAACCAGTGAAGGTTTTGATAAAATGTACTACGACTATTTTCCGGATGCAGCCAAAATGATTCCGAAATGT
AGCACCCAGCTGAAAGCAGTTACCGCACATTTTCAGACCCATACCACCCCGATTCTGCTGAGCAATAACTTTATTG
AACCGCTGGAAATCACCAAAGAGATCTACGATCTGAATAACCCGGAAAAAGAGCCGAAAAAATTCCAGACCGCATA
TGCAAAAAAAACCGGTGATCAGAAAGGTTATCGTGAAGCGCTGTGTAAATGGATTGATTTCACCCGTGATTTTCTG
AGCAAATACACCAAAACCACCAGTATCGATCTGAGCAGCCTGCGTCCGAGCAGCCAGTATAAAGATCTGGGCGAAT
ATTATGCAGAACTGAATCCGCTGCTGTATCATATTAGCTTTCAGCGTATTGCCGAGAAAGAAATCATGGACGCAGT
TGAAACCGGTAAACTGTACCTGTTCCAGATCTACAATAAAGATTTTGCCAAAGGCCATCATGGCAAACCGAATCTG
CATACCCTGTATTGGACCGGTCTGTTTAGCCCTGAAAATCTGGCAAAAACCTCGATTAAACTGAATGGTCAGGCGG
AACTGTTTTATCGTCCGAAAAGCCGTATGAAACGTATGGCACATCGTCTGGGTGAAAAAATGCTGAACAAAAAACT
GAAAGACCAGAAAACCCCGATCCCGGATACACTGTATCAAGAACTGTATGATTATGTGAACCATCGTCTGAGCCAT
GATCTGAGTGATGAAGCACGTGCCCTGCTGCCGAATGTTATTACCAAAGAAGTTAGCCACGAGATCATTAAAGATC
GTCGTTTTACCAGCGACAAATTCCTGTTTCATGTGCCGATTACCCTGAATTATCAGGCAGCAAATAGCCCGAGCAA
ATTTAACCAGCGTGTTAATGCATATCTGAAAGAACATCCAGAAACGCCGATTATTGGTATTGATCGTGGTGAACGT
AACCTGATTTATATCACCGTTATTGATAGCACCGGCAAAATCCTGGAACAGCGTAGCCTGAATACCATTCAGCAGT
TTGATTACCAGAAAAAACTGGATAATCGCGAGAAAGAACGTGTTGCAGCACGTCAGGCATGGTCAGTTGTTGGTAC
AATTAAAGACCTGAAACAGGGTTATCTGAGCCAGGTTATTCATGAAATTGTGGATCTGATGATTCACTATCAGGCC
GTTGTTGTGCTGGAAAACCTGAATTTTGGCTTTAAAAGCAAACGTACCGGCATTGCAGAAAAAGCAGTTTATCAGC
AGTTCGAGAAAATGCTGATTGACAAACTGAATTGCCTGGTGCTGAAAGATTATCCGGCTGAAAAAGTTGGTGGTGT
TCTGAATCCGTATCAGCTGACCGATCAGTTTACCAGCTTTGCAAAAATGGGCACCCAGAGCGGATTTCTGTTTTAT
GTTCCGGCACCGTATACGAGCAAAATTGATCCGCTGACCGGTTTTGTTGATCCGTTTGTTTGGAAAACCATCAAAA
ACCATGAAAGCCGCAAACATTTTCTGGAAGGTTTCGATTTTCTGCATTACGACGTTAAAACGGGTGATTTCATCCT
GCACTTTAAAATGAATCGCAATCTGAGTTTTCAGCGTGGCCTGCCTGGTTTTATGCCTGCATGGGATATTGTGTTT
GAGAAAAACGAAACACAGTTCGATGCAAAAGGCACCCCGTTTATTGCAGGTAAACGTATTGTTCCGGTGATTGAAA
ATCATCGTTTCACCGGTCGTTATCGCGATCTGTATCCGGCAAATGAACTGATCGCACTGCTGGAAGAGAAAGGTAT
TGTTTTTCGTGATGGCTCAAACATTCTGCCGAAACTGCTGGAAAATGATGATAGCCATGCAATTGATACCATGGTT
GCACTGATTCGTAGCGTTCTGCAGATGCGTAATAGCAATGCAGCAACCGGTGAAGATTACATTAATAGTCCGGTTC
GTGATCTGAATGGTGTTTGTTTTGATAGCCGTTTTCAGAATCCGGAATGGCCGATGGATGCAGATGCAAATGGTGC
ATATCATATTGCACTGAAAGGACAGCTGCTGCTGAACCACCTGAAAGAAAGCAAAGATCTGAAACTGCAAAACGGC
ATTAGCAATCAGGATTGGCTGGCATATATCCAAGAACTGCGTAAC
Mutant M537R/F870L AsCas12a amino acid sequence
SEQ ID NO: 20
MTQFEGETNLYQVSKTLRFELIPQGKTLKHIQEQGFIEEDKARNDHYKELKPIIDRIYKTYADQCLQLVQLDWENL
SAAIDSYRKEKTEETRNALIEEQATYRNAIHDYFIGRTDNLTDAINKRHAEIYKGLFKAELFNGKVLKQLGTVTTT
EHENALLRSFDKFTTYFSGFYENRKNVFSAEDISTAIPHRIVQDNFPKFKENCHIFTRLITAVPSLREHFENVKKA
IGIFVSTSIEEVFSFPFYNQLLTQTQIDLYNQLLGGISREAGTEKIKGLNEVLNLAIQKNDETAHIIASLPHRFIP
LFKQILSDRNTLSFILEEFKSDEEVIQSFCKYKTLLRNENVLETAEALFNELNSIDLTHIFISHKKLETISSALCD
HWDTLRNALYERRISELTGKITKSAKEKVQRSLKHEDINLQEIISAAGKELSEAFKQKTSEILSHAHAALDQPLPT
TLKKQEEKEILKSQLDSLLGLYHLLDWFAVDESNEVDPEFSARLTGIKLEMEPSLSFYNKARNYATKKPYSVEKFK
LNFQRPTLASGWDVNKEKNNGAILFVKNGLYYLGIMPKQKGRYKALSFEPTEKTSEGFDKMYYDYFPDAAKMIPKC
STQLKAVTAHFQTHTTPILLSNNFIEPLEITKEIYDLNNPEKEPKKFQTAYAKKTGDQKGYREALCKWIDFTRDFL
SKYTKTTSIDLSSLRPSSQYKDLGEYYAELNPLLYHISFQRIAEKEIMDAVETGKLYLFQIYNKDFAKGHHGKPNL
HTLYWTGLFSPENLAKTSIKLNGQAELFYRPKSRMKRMAHRLGEKMLNKKLKDQKTPIPDTLYQELYDYVNHRLSH
DLSDEARALLPNVITKEVSHEIIKDRRFTSDKFLFHVPITLNYQAANSPSKFNQRVNAYLKEHPETPIIGIDRGER
NLIYITVIDSTGKILEQRSLNTIQQFDYQKKLDNREKERVAARQAWSVVGTIKDLKQGYLSQVIHEIVDLMIHYQA
VVVLENLNFGFKSKRTGIAEKAVYQQFEKMLIDKLNCLVLKDYPAEKVGGVLNPYQLTDQFTSFAKMGTQSGFLFY
VPAPYTSKIDPLTGFVDPFVWKTIKNHESRKHFLEGFDFLHYDVKTGDFILHFKMNRNLSFQRGLPGFMPAWDIVE
EKNETQFDAKGTPFIAGKRIVPVIENHRFTGRYRDLYPANELIALLEEKGIVFRDGSNILPKLLENDDSHAIDTMV
ALIRSVLQMRNSNAATGEDYINSPVRDLNGVCFDSRFQNPEWPMDADANGAYHIALKGQLLLNHLKESKDLKLONG
ISNQDWLAYIQELRN

### Example 2

Overexpression and Purification of LbCas12a Mutants in E. coli Cells

This example demonstrates the over expression and purification of seven Cas12a mutants, N527R, D559P, E795L, N527R/D559P, D559P/E795L, N527R/E795L and N527R/D559P/E795L. The LbCas12a mutants were introduced by site-directed mutagenesis, using standard PCR conditions and primers (Table 1). After transformation into E. coli BL21 (DE3) cells, a colony with the appropriate strain was used to inoculate TB media with kanamycin (0.05 mg/mL) and grown at 37° C. until an OD of approximately 0.9 was reached, then the flask was cooled to 18° C. for 30 minutes. The addition of 1 M IPTG (500 μL) was used to induce protein expression, followed by growth at 18° C. for 19 hours. Cells were harvested and the cell pellet was re-suspended and lysed on an Avestin Emulsiflex C3 pre-chilled to 4° C. at 15-20 kpsi with three passes. The lysate was centrifuged at 16,000×g for 20 minutes at 4° C. to remove cell debris

The cleared lysate was put over a HisTrap HP column. The procedure consisted of equilibrating the resin with His-Bind buffer (20 mM NaPO₄ pH 6.8, 0.5 M NaCl, 10 mM imidazole, 5% glycerol), followed by sample loading. The sample was washed with His-Bind buffer, followed by an additional standard wash and a 10% “B” wash consisting of 10% His-Elution buffer (10 mM NaPO₄ pH 6.8, 250 mM NaCl, 150 mM imidazole, 5% glycerol). Finally, the sample was eluted using His-Elution buffer. The LbCas12a mutants were then put over a HiTrap Heparin HP column. The procedure consisted of equilibrating the resin with the Heparin-Bind buffer (20 mM NaPO₄ pH 6.8, 250 mM NaCl, 10% glycerol), followed by sample loading. The sample was then washed with Heparin-Bind buffer, followed by a 5% “B” wash consisting of 5% Heparin Elution buffer (10 mM NaPO₄ pH 6.8, 1 M NaCl, 10% glycerol). Finally, the purified protein was eluted using Heparin Elution buffer (10 mM NaPO₄, pH 6.8, 1 M NaCl, 10% glycerol). Finally, the purified protein was eluted using Heparin.

The purified LbCas12a mutants were concentrated to approximately 10 mg/mL and stored at −20° C. in storage buffer (25 mM Tris-HCl PH 7.4, 0.3 M NaCl). 1 mM EDTA, 1 mM DTT, 50% glycerol).

TABLE 1
Sequences of primers use for site-directed mutagenesis with the amino acid codon
underlined. All primers ordered as DNA oligos from Integrated DNA Technologies.

| Primer | | SEQ |
|---|---|---|
| Name | Primer Sequence (5′-3′) | ID NO. |
| LbCas12a | GCAAAGACAAGTTCAAACTGTACTTTCAGCGTCCGCAGTTTATGGGTGGTTGG | 21 |
| N527R Fwd | | |
| LbCas12a | CCAACCACCCATAAACTGCGGACGCTGAAAGTACAGTTTGAACTTGTCTTTGC | 22 |
| N527R Rev | | |
| LbCas12a | TATGGTAGTAAATACTATCTGGCCATCATGCCGAAAAAATACGCAAAATGCCTGCAGA | 23 |
| D559P Fwd | | |
| LbCas12a | TCTGCAGGCATTTTGCGTATTTTTTCGGCATGATGGCCAGATAGTATTTACTACCATA | 24 |
| D559P Rev | | |
| LbCas12a | ACAAACGTTTTAGCGAGGATCAGTATCTGCTGCATATCCCGATTGCCATCA | 25 |
| E795L Fwd | | |
| LbCas12a | TGATGGCAATCGGGATATGCAGCAGATACTGATCCTCGCTAAAACGTTTGT | 26 |
| E795L Rev | | |

### Example 3

Novel LbCas12a Substitution Mutants Enhance the Cleavage Activity in a Human Cell Line Based Activity Assay when Delivered Into Human Cells Via Ribonucleoprotein Complex.

The following example demonstrates the ability of LbCas12a mutants to improve genome editing efficiency when delivered as an RNP complex. The example demonstrates the ability of LbCas12a mutants to show comparable genome editing efficiency when delivered at a high dose and increased genome editing efficiency when delivered at a low dose by ribonucleoprotein (RNP) complex into human cells with electroporation transfection.

The RNP complex was formed by incubating purified LbCas12a and sgRNA (Table 2, entries 3 and 9) at a ratio of 1:1.2 in PBS buffer for 10 minutes at room temperature. The RNP complexes (5 μM final dose) were transfected into HEK293 immortalized human cells using a Lonza 4D-Nucleofector™ and Amaxa® 96-well Shuttle Device with Alt-R® Cpf1 Electroporation Enhancer (3 μM, Integrated DNA Technologies). The experiments were performed in biological triplicate and after 48 hours at 37° C., adherent cells were lysed with QuickExtract™ DNA extraction solution (50 μL).

TABLE 2
Sequences of Cas12 (Cpfl) sgRNA used in genome editing of HEK293 cells. All
sRNA ordered as RNA oligos from Integrated DNA Technologies with the AltR®
end modifications on both the 5′ and 3′ ends.

| | | SEQ ID |
|---|---|---|
| sgRNA | sgRNA Sequence (5′-3′) | NO. |
| LbCpfl HRPT | UAAUUUCUACUAAGUGUAGAUAUAGUCUUUCCUUGGGUCUGUUA | 27 |
| 38094-S-23 | | |
| LbCpfl HRPT | UAAUUUCUACUAAGUGUAGAUCUUGGGUGUGUUAAAAGUGACCA | 28 |
| 38104-S-23 | | |
| LbCpfl HRPT | UAAUUUCUACUAAGUGUAGAUACACACCCAAGGAAAGACUAUGA | 29 |
| 38115-AS-23 | | |
| LbCpfl HRPT | UAAUUUCUACUAAGUGUAGAUAUCCGUGCUGAGUGUACCAUGCA | 30 |
| 38146-AS-23 | | |
| LbCpfl HRPT | UAAUUUCUACUAAGUGUAGAUUAAACACUGUUUCAUUUCAUCCG | 31 |
| 38164-AS-23 | | |
| LbCpfl HRPT | UAAUUUCUACUAAGUGUAGAUGAAACGUCAGUCUUCUCUUUUGU | 32 |
| 38164-S-23 | | |
| LbCpfl HRPT | UAAUUUCUACUAAGUGUAGAUUAAUGCCCUGUAGUCUCUCUGUA | 33 |
| 38186-S-23 | | |
| LbCpfl HRPT | UAAUUUCUACUAAGUGUAGAUUAAUUAACAGCUUGCUGGUGAAA | 34 |
| 38228-S-23 | | |
| LbCpfl HRPT | UAAUUUCUACUAAGUGUAGAUGGUUAAAGAUGGUUAAAUGAUUG | 35 |
| 38330-AS-23 | | |
| LbCpfl HRPT | UAAUUUCUACUAAGUGUAGAUUGUGAAAUGGCUUAUAAUUGCUU | 36 |
| 38343-S-23 | | |
| LbCpfl HRPT | UAAUUUCUACUAAGUGUAGAUGUUGUUGGAUUUGAAAUUCCAGA | 37 |
| 38455-S-23 | | |
| LbCpfl HRPT | UAAUUUCUACUAAGUGUAGAUUUGUAGGAUAUGCCCUUGACUAU | 38 |
| 38486-S-23 | | |
| AsCpfl HRPT | UAAUUUCUACUCUUGUAGAUAUAGUCUUUCCUUGGGUGUGU | 39 |
| 38094-S | | |
| AsCpfl HRPT | UAAUUUCUACUCUUGUAGAUCUUGGGUGUGUUAAAAGUGAC | 40 |
| 38104-S | | |
| AsCpfl HRPT | UAAUUUCUACUCUUGUAGAUACACACCCAAGGAAAGACUAU | 41 |
| 38115-AS | | |
| AsCpfl HRPT | UAAUUUCUACUCUUGUAGAUAUCCGUGCUGAGUGUACCAUG | 42 |
| 38146-AS | | |
| AsCpfl HRPT | UAAUUUCUACUCUUGUAGAUUAAACACUGUUUCAUUUCAUC | 43 |
| 38164-AS | | |
| AsCpfl HRPT | UAAUUUCUACUCUUGUAGAUGAAACGUCAGUCUUCUCUUUU | 44 |
| 38164-S | | |
| AsCpfl HRPT | UAAUUUCUACUCUUGUAGAUUAAUGCCCUGUAGUCUCUCUG | 45 |
| 38186-S | | |
| AsCpfl HRPT | UAAUUUCUACUCUUGUAGAUUAAUUAACAGCUUGCUGGUGA | 46 |
| 38228-S | | |
| AsCpfl HRPT | UAAUUUCUACUCUUGUAGAUGGUUAAAGAUGGUUAAAUGAU | 47 |
| 38330-AS | | |
| AsCpfl HRPT | UAAUUUCUACUCUUGUAGAUUGUGAAAUGGCUUAUAAUUGC | 48 |
| 38343-S | | |
| AsCpfl HRPT | UAAUUUCUACUCUUGUAGAUGUUGUUGGAUUUGAAAUUCCA | 49 |
| 38455-S | | |
| AsCpfl HRPT | UAAUUUCUACUCUUGUAGAUUUGUAGGAUAUGCCCUUGACU | 50 |
| 38486-S | | |

Crude lysates were incubated at 65° C. for 15 minutes, followed by heat inactivation at 98° C. for 3 minutes. Crude genomic DNA was diluted 5-fold in TE buffer and used as PCR template. PCR (primers listed in Table 3) was used to amplify 1.2 kbp fragments of the HPRT loci using Q5® DNA Polymerase (New England Biolabs) and the following parameters: 98° C. for 30 sec, followed by 98° C. for 10 sec, 65° C. for 15 sec and 72° C. for 1 min which was repeated 24 times, followed by a final extension at 72° C. for 2 min. Heteroduplexes were formed by the addition of NEBuffer 2 and initially heating to 95° C. for 10 min with a slow cool down to room temperature. The heteroduplexes were then cleaved by 2 U of T7 Endonuclease I (New England Biolabs) for 1 hour at 37° C. The cleavage products were analyzed by capillary electrophoresis (Fragment Analyzer, Advanced Analytical).

TABLE 3
Sequences of primers used for amplification of edited genomic
HEK293 DNA prior to analysis by T7EI. All primers ordered as
DNA oligos from Integrated DNA Technologies.

| Primer Name | Primer Sequence (5′-3′) | SEQ ID NO. |
|---|---|---|
| HRPT low GC Fwd | AAGAAGTTGTGATAAAAGGTGATGCT | 51 |
| HRPT low GC Rev | ACACATCCATGGGACTTCTGCCTC | 52 |

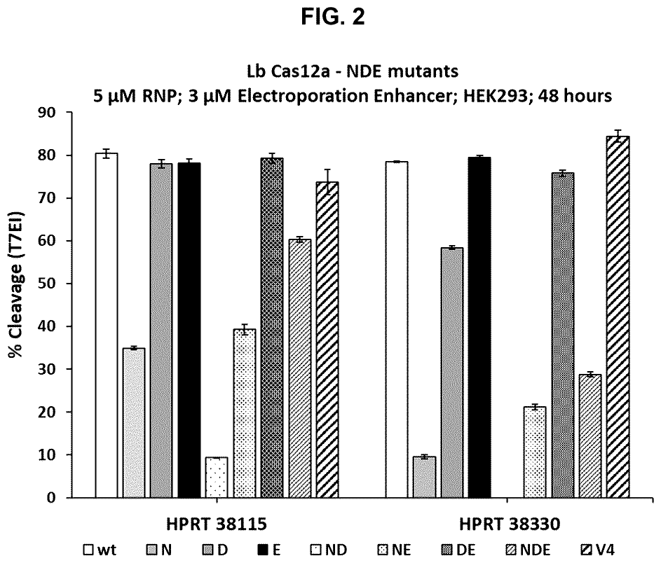

The endonuclease activity of wild type and mutant LbCas12a in HEK293 human cells are described in FIG. 2 and Table 4. RNP delivery of LbCas12a mutants D559P, E795L and D559P/E795L resulted in similar activity as wild type LbCas12a and AsCas12a-M537R/F870L (˜80% cleavage). As this initial screen was to determine activity at the highest dose (5 μM), a dose response with lower concentrations (2, 1 and 0.05 μM) of RNP was preformed to determine if these mutants can instill enhanced activity.

TABLE 4a
Endonuclease activity of LbCas12 mutants as compared to wild type
LbCas12 and AsCas12a-M537R/F870L after 48 hours in HEK293 human
cells at HRPT-38115. Values calculated as percent cleavage

| | Replicate | Replicate | Replicate | Aver- | Std |
|---|---|---|---|---|---|
| Cas12a | 1 | 2 | 3 | age | Dev |
| LbCas12a- | 78.9 | 80.7 | 81.5 | 80.35 | 1.07 |
| wild type | | | | | |
| LbCas12a- | 35.5 | 34.9 | 34.5 | 34.96 | 0.43 |
| N527R | | | | | |
| LbCas12a- | 78.2 | 76.6 | 79 | 77.96 | 0.99 |
| D559P | | | | | |
| LbCas12a- | 77.5 | 79.6 | 77.5 | 78.16 | 0.99 |
| E795L | | | | | |
| LbCas12a- | 9.3 | 9.3 | 9.5 | 9.39 | 0.06 |
| N527R/D559P | | | | | |
| LbCas12a- | 41 | 39.1 | 38.9 | 39.33 | 1.22 |
| N527R/E795L | | | | | |
| LbCas12a- | 80.3 | 77.8 | 79.7 | 79.28 | 1.09 |
| D559P/E795L | | | | | |
| LbCas12a- | 59.4 | 61 | 60.5 | 60.32 | 0.69 |
| N527R/D559P/ | | | | | |
| E795L | | | | | |
| AsCas12a- | 70.8 | — | 76.6 | 73.7 | 2.89 |
| M537R/F870L | | | | | |

TABLE 4b
Endonuclease activity of LbCas12 mutants as compared to wild type
LbCas12 and AsCas12a-M537R/F870L after 48 hours in HEK293 human
cells at HRPT-38330. Values calculated as percent cleavage

| | Replicate | Replicate | Replicate | Aver- | Std |
|---|---|---|---|---|---|
| Cas12a | 1 | 2 | 3 | age | Dev |
| LbCas12a- | 78.1 | 78.6 | 78.7 | 78.5 | 0.2 |
| wild type | | | | | |
| LbCas12a- | 10.1 | 9.7 | 9.0 | 9.6 | 0.5 |
| N527R | | | | | |
| LbCas12a- | 58.0 | 58.0 | 59.2 | 58.4 | 0.5 |
| D559P | | | | | |
| LbCas12a- | 78.9 | 79.6 | 79.9 | 79.5 | 0.4 |
| E795L | | | | | |
| LbCas12a- | 0.0 | 0.0 | 0.0 | 0.0 | 0.0 |
| N527R/D559P | | | | | |
| LbCas12a- | 20.4 | 22.0 | 21.1 | 21.2 | 0.7 |
| N527R/E795L | | | | | |
| LbCas12a- | 76.7 | 75.6 | 75.0 | 75.8 | 0.7 |
| D559P/E795L | | | | | |
| LbCas12a- | 28.2 | 29.4 | 28.8 | 28.8 | 0.5 |
| N527R/D559P/ | | | | | |
| E795L | | | | | |
| AsCas12a- | 86.2 | 82.9 | 84.0 | 84.4 | 1.4 |
| M537R/F870L | | | | | |

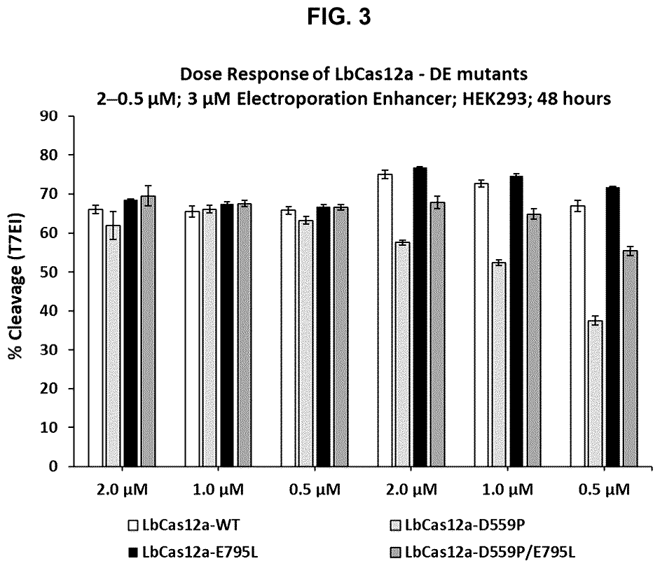

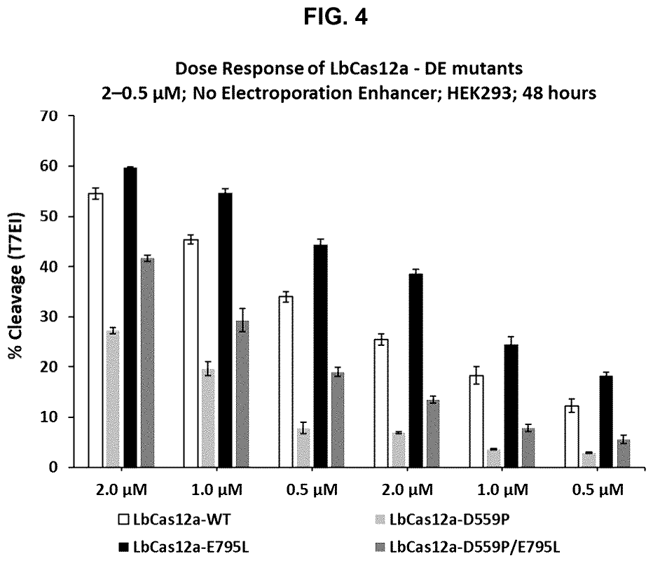

The dose response was achieved as described above, reducing the amount of RNP by 2-fold increments (with and without Alt-R® Electroporation Enhancer) and beginning with a 2 μM dose. The results are shown in FIG. 3-4 and Tables 5-6. RNP delivery of LbCas12a requires the addition of Alt-R® Cpf1 Electroporation Enhancer for maximum cutting efficiency. At these doses, LbCas12a mutants displayed either similar or a slight increase in activity as compared to wild type; therefore, new sites and even lower doses needed to be investigated to show differences in activity.

TABLE 5a
Endonuclease activity of LbCas12 mutants as compared to
wild type LbCas12 after 48 hours in HEK293 human cells at HRPT-38115
with Alt-R ® Electroporation Enhancer. Values calculated as percent cleavage

| | Dose | | | | | |
|---|---|---|---|---|---|---|
| Cas12a | (μM) | Replicate 1 | Replicate 2 | Replicate 3 | Average | Std Dev |
| LbCas12a-wild type | 2.0 | 67.6 | 65.1 | 65.5 | 66.06 | 1.11 |
| | 1.0 | 67.4 | 65.3 | 63.7 | 65.49 | 1.49 |
| | 0.5 | 66.9 | 34.5 | 66.0 | 65.81 | 1.01 |
| LbCas12a-D559P | 2.0 | 66.9 | 58.6 | 60.4 | 61.95 | 3.59 |
| | 1.0 | 64.7 | 67.1 | 66.6 | 66.10 | 1.04 |
| | 0.5 | 61.8 | 63.9 | 63.9 | 63.22 | 0.98 |
| LbCas12a-E795L | 2.0 | 68.4 | 68.8 | 68.5 | 68.58 | 0.15 |
| | 1.0 | 66.7 | 68.1 | 67.5 | 67.45 | 0.59 |
| | 0.5 | 66.1 | 66.5 | 67.6 | 66.72 | 0.67 |
| LbCas12a-D559P/E795L | 2.0 | 73.1 | 66.9 | 68.5 | 69.49 | 2.61 |
| | 1.0 | 67.5 | 66.6 | 68.5 | 67.53 | 0.79 |
| | 0.5 | 67.5 | 66.3 | 65.9 | 66.58 | 0.68 |

TABLE 5b
Endonuclease activity of LbCas12 mutants as compared to
wild type LbCas12 after 48 hours in HEK293 human cells at HRPT-38330
with Alt-R ® Electroporation Enhancer. Values calculated as percent cleavage

| | Dose | | | | | |
|---|---|---|---|---|---|---|
| Cas12a | (μM) | Replicate 1 | Replicate 2 | Replicate 3 | Average | Std Dev |
| LbCas12a-wild type | 2.0 | 76.5 | 74.5 | 74.2 | 75.06 | 1.06 |
| | 1.0 | 73.8 | 71.5 | 72.9 | 72.73 | 0.96 |
| | 0.5 | 65.0 | 68.6 | 67.2 | 66.95 | 1.49 |
| LbCas12a-D559P | 2.0 | 57.1 | 58.4 | 57.2 | 57.55 | 0.60 |
| | 1.0 | 52.8 | 53.0 | 51.3 | 52.34 | 0.75 |
| | 0.5 | 38.1 | 35.8 | 38.5 | 37.48 | 1.19 |
| LbCas12a-E795L | 2.0 | 76.9 | 76.7 | 76.9 | 76.84 | 0.10 |
| | 1.0 | 74.7 | 75.3 | 73.9 | 74.63 | 0.55 |
| | 0.5 | 72.0 | 71.7 | 71.5 | 71.72 | 0.18 |
| LbCas12a-D559P/E795L | 2.0 | 65.6 | 69.4 | 68.6 | 67.88 | 1.63 |
| | 1.0 | 65.1 | 66.3 | 63.1 | 64.83 | 1.31 |
| | 0.5 | 56.9 | 55.2 | 54.1 | 55.39 | 1.17 |

TABLE 6a
Endonuclease activity of LbCas12 mutants as compared
to wild type LbCas12 after 48 hours in HEK293 human
cells at HRPT-38115 without Alt-R ® Electroporation
Enhancer. Values calculated as percent cleavage

| | Dose | Replicate | Replicate | Replicate | Aver- | Std |
|---|---|---|---|---|---|---|
| Cas12a | (μM) | 1 | 2 | 3 | age | Dev |
| LbCas12a- | 2.0 | 56.2 | 54.1 | 53.5 | 54.60 | 1.13 |
| wild type | 1.0 | 46.0 | 44.0 | 46.1 | 45.38 | 0.96 |
| | 0.5 | 35.5 | 35.4 | 32.9 | 33.94 | 10.5 |
| LbCas12a- | 2.0 | 26.5 | 27.2 | 28.1 | 27.24 | 0.66 |
| D559P | 1.0 | 21.5 | 18.6 | 18.7 | 19.61 | 1.34 |
| | 0.5 | 6.3 | 9.2 | 7.9 | 7.81 | 1.16 |
| LbCas12a- | 2.0 | 59.9 | 59.4 | 59.7 | 59.67 | 0.18 |
| E795L | 1.0 | 55.6 | 53.8 | 54.7 | 54.72 | 0.73 |
| | 0.5 | 43.0 | 45.8 | 44.4 | 44.39 | 1.13 |
| LbCas12a- | 2.0 | 41.6 | 42.4 | 40.9 | 41.64 | 0.63 |
| D559P/ | 1.0 | 32.3 | 29.1 | 26.6 | 29.33 | 2.32 |
| E795L | 0.5 | 20.3 | 18.7 | 18.0 | 18.99 | 0.97 |

TABLE 6b
Endonuclease activity of LbCas12 mutants as compared to wild
type LbCas12 after 48 hours in HEK293 human cells at HRPT-38330
without Alt-R ® Electroporation Enhancer. Values calculated as percent cleavage

| | Dose | | | | | |
|---|---|---|---|---|---|---|
| Cas12a | (μM) | Replicate 1 | Replicate 2 | Replicate 3 | Average | Std Dev |
| LbCas12a-wild type | 2.0 | 24.9 | 24.4 | 27.1 | 25.49 | 1.18 |
| | 1.0 | 18.4 | 20.3 | 16.0 | 18.26 | 1.75 |
| | 0.5 | 14.2 | 11.0 | 11.6 | 12.27 | 1.36 |
| LbCas12a-D559P | 2.0 | 18.4 | 7.0 | 6.6 | 6.86 | 0.21 |
| | 1.0 | 14.2 | 3.5 | 3.8 | 3.62 | 0.13 |
| | 0.5 | 18.4 | 2.7 | 2.9 | 2.85 | 0.10 |
| LbCas12a-E795L | 2.0 | 38.1 | 38.1 | 39.8 | 38.66 | 0.82 |
| | 1.0 | 26.8 | 23.5 | 23.2 | 24.48 | 1.63 |
| | 0.5 | 19.2 | 18.0 | 17.4 | 18.21 | 0.72 |
| LbCas12a-D559P/E795L | 2.0 | 12.8 | 13.2 | 14.5 | 13.47 | 0.74 |
| | 1.0 | 6.8 | 7.9 | 8.7 | 7.82 | 0.77 |
| | 0.5 | 5.3 | 4.6 | 6.7 | 5.53 | 0.90 |

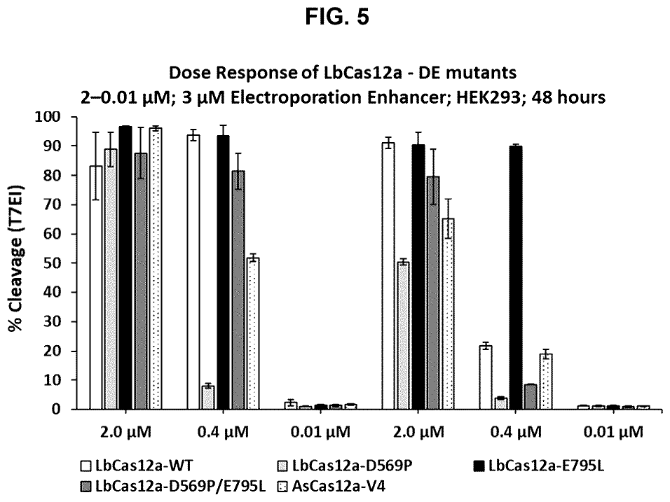

The final dose response in this example was set up as described above, reducing the amount of RNP even further by 5-fold increments, starting with a 2 μM dose. The RNP was formed using sgRNA 2 and 8 for the LbCas12a RNP and 14 and 20 for the AsCas12a RNP (Table 1). The results are shown in FIG. 5 and Table 7. RNP delivery of LbCas12a-E795L showed increased activity (˜90% cleavage) as compared to wild type LbCas12a (˜22% cleavage) and AsCas12a-M537R/F870L (˜19% cleavage) at the 38228 site of the HPRT loci at the low of 0.4 μM and retained the high cleavage activity (˜90% cleavage) at the 38104 site as seen by the wild type LbCas12a. The single E795L mutant of the LbCas12a nuclease increased genome editing activity up to 4.5-fold at the lowest dose (0.4 μM).

TABLE 7a
Endonuclease activity of LbCas12 mutants as compared to
wild type LbCas12 and AsCas12a-M537R/F870L after 48 hours in
HEK293 human cells at HRPT-38104. Values calculated as percent cleavage.

| | Dose | | | | | |
|---|---|---|---|---|---|---|
| Cas12a | (μM) | Replicate 1 | Replicate 2 | Replicate 3 | Average | Std Dev |
| LbCas12a-wild type | 2.0 | 76.0 | 99.4 | 74.2 | 83.22 | 11.50 |
| | 0.4 | 96.4 | 93.1 | 91.6 | 93.72 | 1.99 |
| | 0.01 | 2.5 | 3.7 | 1.9 | 2.68 | 0.74 |
| LbCas12a-D559P | 2.0 | 94.4 | 91.3 | 80.7 | 88.81 | 5.89 |
| | 0.4 | 9.0 | 8.1 | 6.8 | 7.96 | 0.88 |
| | 0.01 | 1.3 | 1.2 | 1.3 | 1.29 | 0.03 |
| LbCas12a-E795L | 2.0 | 96.5 | 96.1 | 96.9 | 96.51 | 0.32 |
| | 0.4 | 96.1 | 95.8 | 88.6 | 93.49 | 3.48 |
| | 0.01 | 1.7 | 2.1 | 1.7 | 1.81 | 0.18 |
| LbCas12a-D559P/E795L | 2.0 | 94.7 | 92.7 | 75.4 | 87.59 | 8.64 |
| | 0.4 | 87.5 | 75.3 | — | 81.39 | 6.11 |
| | 0.01 | 1.5 | 1.4 | 2.0 | 1.62 | 0.28 |
| AsCas12a-M537R/F870L | 2.0 | 100.0 | 96.6 | 96.7 | 97.78 | 1.57 |
| | 0.4 | 52.0 | 53.4 | 50.1 | 51.82 | 1.33 |
| | 0.01 | 2.1 | 1.8 | 1.9 | 1.92 | 0.11 |

TABLE 7b
Endonuclease activity of LbCas12 mutants as compared to
wild type LbCas12 and AsCas12a-M537R/F870L after 48 hours in
HEK293 human cells at HRPT-38228. Values calculated as percent cleavage

| | Dose | | | | | |
|---|---|---|---|---|---|---|
| Cas12a | (μM) | Replicate 1 | Replicate 2 | Replicate 3 | Average | Std Dev |
| LbCas12a-wild type | 2.0 | 90.1 | 93.7 | 89.4 | 91.06 | 1.86 |
| | 0.4 | 20.7 | — | 23.0 | 21.85 | 1.16 |
| | 0.01 | 1.4 | 1.6 | 1.7 | 1.55 | 0.12 |
| LbCas12a-D559P | 2.0 | 49.3 | 51.5 | — | 50.42 | 1.09 |
| | 0.4 | 4.5 | 3.6 | 3.4 | 3.80 | 0.48 |
| | 0.01 | 1.4 | 1.5 | 1.3 | 1.40 | 0.07 |
| LbCas12a-E795L | 2.0 | 84.1 | 94.0 | 92.6 | 90.24 | 4.36 |
| | 0.4 | 90.6 | 89.0 | — | 89.79 | 0.79 |
| | 0.01 | 1.4 | 1.3 | 1.6 | 1.47 | 0.12 |
| LbCas12a-D559P/E795L | 2.0 | 83.3 | 66.5 | 88.8 | 79.54 | 9.48 |
| | 0.4 | 8.3 | 8.4 | 8.6 | 8.44 | 0.14 |
| | 0.01 | 1.6 | 1.1 | 1.3 | 1.37 | 0.20 |
| AsCas12a-M537R/F870L | 2.0 | 72.7 | 56.3 | 66.2 | 65.10 | 6.73 |
| | 0.4 | 19.8 | 20.5 | 16.7 | 19.02 | 1.66 |
| | 0.01 | 1.3 | 1.8 | 1.4 | 1.52 | 0.23 |

### Example 4

Single LbCas12a Substitution Mutant Enhances the Cleavage Activity in a Human Cell Line Based Activity Assay when Delivered Into Human Cells Via Ribonucleoprotein Complex at Low Doses.

The following example demonstrates the ability of mutant E795L LbCas12a to show increased genome editing efficiency when delivered at low doses by RNP complex into human cells with electroporation transfection. That this invention increases genome editing efficiency when wild-type or mutant Cas12a is delivered into human cells as an RNP complex.

The RNP complex was formed by incubating purified Cas12a and sgRNA (Table 2, entries 1-12 for the LbCas12a RNP and entries 13-24 for the AsCas12a RNP) at a ratio of 1:1.2 in PBS buffer for 10 minutes at room temperature. The RNP complexes (1, 0.22, 0.05 and 0.01 μM final doses) were transfected into HEK293 immortalized human cells using a Lonza 4D-Nucleofector™ and Amaxa® 96-well Shuttle Device with Alt-R® Cpf1 Electroporation Enhancer (3 μM, Integrated DNA Technologies). The experiments were performed in biological duplicate and after 48 hours at 37° C., adherent cells were lysed with QuickExtract™ DNA extraction solution (50 μL).

Crude lysates were incubated at 65° C. for 15 minutes, followed by heat inactivation at 98° C. for 3 minutes. Crude genomic DNA was diluted 15-fold in TE buffer and used as PCR template. PCR (primers listed in Table 3) was used to amplify 1.2 kbp fragments of the HPRT loci using Q5® DNA Polymerase (New England Biolabs) and the following parameters: 98° C. for 30 sec, followed by 98° C. for 10 sec, 65° C. for 15 sec and 72° C. for 1 min which was repeated 24 times, followed by a final extension at 72° C. for 2 min. Heteroduplexes were formed by the addition of NEBuffer 2 and initially heating to 95° C. for 10 min with a slow cool down to room temperature. The heteroduplexes were then cleaved by 2 U of T7 Endonuclease I (New England Biolabs) for 1 hour at 37° C. The cleavage products were analyzed by capillary electrophoresis (Fragment Analyzer, Advanced Analytical).

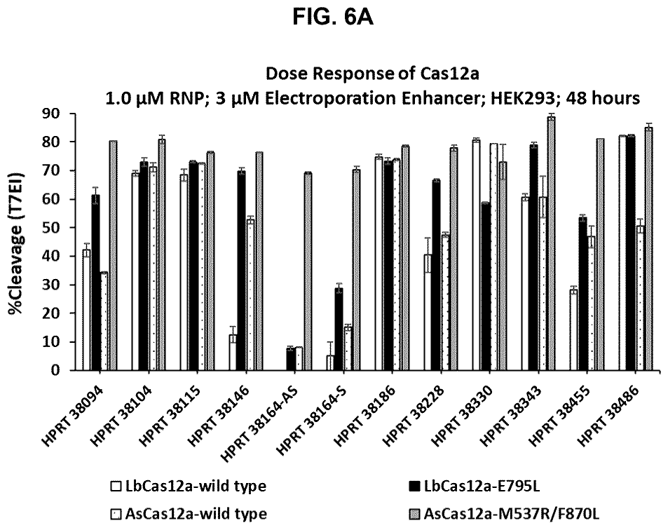

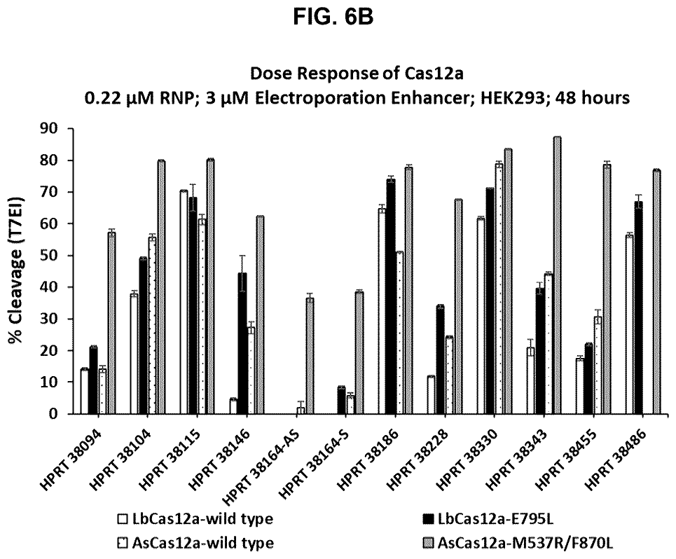

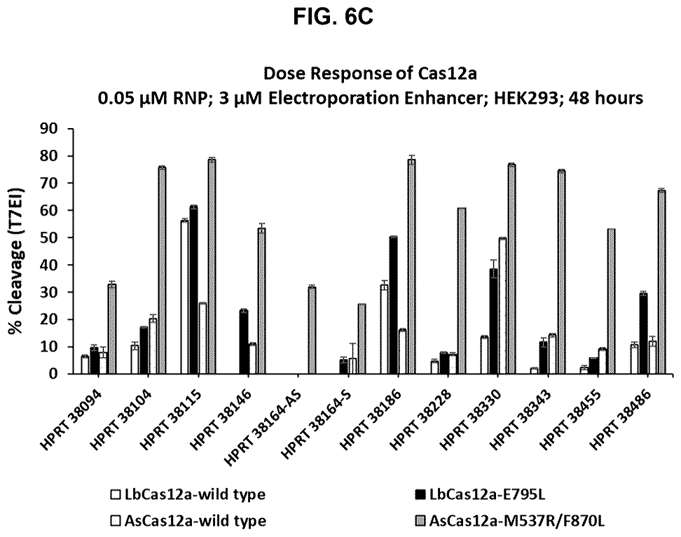

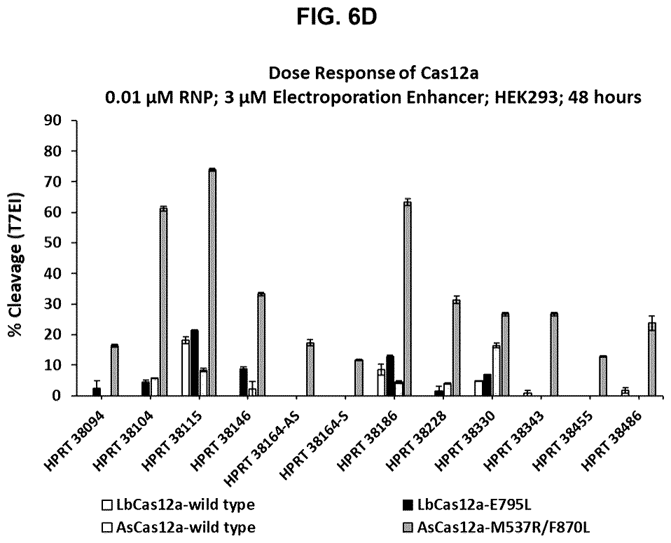

The endonuclease activity of wild type and mutant LbCas12a as compared to wild type and mutant AsCas12a in HEK293 human cells are described in FIG. 6A, FIG. 6b, FIG. 6C, FIG. 6D and Table 8. Overall, RNP delivery of LbCas12a-E795L showed improved activity as compared to wild type Lb- and AsCas12a. The 0.05 μM dose shows the increased activity of the enzyme even at low doses (FIG. 6c). At this dose, LbCas12a-E795L exceeds wild type LbCas12a's activity up to 23-fold at the HPRT 38146 site (Table 8, entries 51 and 55) and wild type AsCas12a's activity up to 3-fold at the HPRT 38186 site (Table 8, entries 103 and 107). At the highest distinguishable dose (0.22 μM, FIG. 6b) for the LbCas12a variants, the E795L mutant exhibited increased activity over the wild type version up to 11-fold at the HPRT 38146 site (Table 8, entries 50 and 54) and almost 2-fold at the same site against the wild-type AsCas12a (Table 8, entries 54 and 58).

TABLE 8
Endonuclease activity of LbCas12a-E795L compared
to wild type Lb- and AsCas12a and M AsCas12a-537R/F870L
after 48 hours in HEK293 human cells at HRPT-38228.
Values calculated as percent cleavage.

| | | Dose | Replicate | Replicate | Aver- | Std | |
|---|---|---|---|---|---|---|---|
| Cas12a | Site | (μM) | 1 | 2 | age | Dev | Entry |
| LbCas12a- | HPRT- | 1.00 | 39.8 | 44.5 | 42.2 | 2.36 | 1 |
| wild type | 38094- | 0.22 | 13.7 | 14.6 | 14.2 | 0.45 | 2 |
| | S | 0.05 | 5.8 | 7.0 | 6.4 | 0.60 | 3 |
| | | 0.01 | 0.0 | 0.0 | 0.0 | 0.00 | 4 |
| LbCas12a- | HPRT- | 1.00 | 64.2 | 58.4 | 61.3 | 2.90 | 5 |
| E795L | 38094- | 0.22 | 21.5 | 20.9 | 21.2 | 0.30 | 6 |
| | S | 0.05 | 8.6 | 10.9 | 9.8 | 1.15 | 7 |
| | | 0.01 | 4.8 | 0.0 | 2.4 | 2.40 | 8 |
| AsCas12a- | HPRT- | 1.00 | 34.6 | 33.9 | 34.3 | 0.35 | 9 |
| wild type | 38094- | 0.22 | 12.9 | 15.2 | 14.1 | 1.15 | 10 |
| | S | 0.05 | 10.1 | 6.0 | 8.1 | 2.05 | 11 |
| | | 0.01 | 0.0 | 0.0 | 0.0 | 0.00 | 12 |
| AsCas12a- | HPRT- | 1.00 | 80.3 | 80.2 | 80.3 | 0.05 | 13 |
| M537R/ | 38094- | 0.22 | 58.4 | 55.9 | 57.2 | 1.25 | 14 |
| F870L | S | 0.05 | 34.2 | 31.7 | 33.0 | 1.25 | 15 |
| | | 0.01 | 16.9 | 16.0 | 16.5 | 0.54 | 16 |
| LbCas12a- | HPRT- | 1.00 | 67.9 | 70.0 | 69.0 | 1.05 | 17 |
| wild type | 38104- | 0.22 | 36.8 | 38.8 | 37.8 | 1.00 | 18 |
| | S | 0.05 | 11.8 | 9.1 | 10.5 | 1.35 | 19 |
| | | 0.01 | 0.0 | 0.0 | 0.0 | 0.00 | 20 |
| LbCas12a- | HPRT- | 1.00 | 74.5 | 71.4 | 73.0 | 1.55 | 21 |
| E795L | 38104- | 0.22 | 49.5 | 48.6 | 49.1 | 0.45 | 22 |
| | S | 0.05 | 17.5 | 17.0 | 17.3 | 0.25 | 23 |
| | | 0.01 | 5.1 | 3.9 | 4.5 | 0.60 | 24 |
| AsCas12a- | HPRT- | 1.00 | 72.8 | 69.6 | 71.2 | 1.60 | 25 |
| wild type | 38104- | 0.22 | 54.5 | 56.7 | 55.6 | 1.10 | 26 |
| | S | 0.05 | 21.8 | 19.0 | 20.4 | 1.40 | 27 |
| | | 0.01 | 5.6 | 5.9 | 5.8 | 0.15 | 28 |
| AsCas12a- | HPRT- | 1.00 | 79.4 | 82.4 | 80.9 | 1.50 | 29 |
| M537R/ | 38104- | 0.22 | 80.2 | 79.5 | 79.9 | 0.35 | 30 |
| F870L | S | 0.05 | 75.0 | 76.3 | 75.7 | 0.65 | 31 |
| | | 0.01 | 62.0 | 60.4 | 61.2 | 0.80 | 32 |
| LbCas12a- | HPRT- | 1.00 | 66.2 | 70.6 | 68.4 | 2.20 | 33 |
| wild type | 38115- | 0.22 | 70.1 | 70.6 | 70.4 | 0.25 | 34 |
| | AS | 0.05 | 55.8 | 56.9 | 56.4 | 0.55 | 35 |
| | | 0.01 | 19.3 | 17.1 | 18.2 | 1.10 | 36 |
| LbCas12a- | HPRT- | 1.00 | 73.5 | 72.6 | 73.1 | 0.45 | 37 |
| E795L | 38115- | 0.22 | 72.5 | 64.0 | 68.3 | 4.25 | 38 |
| | AS | 0.05 | 62.0 | 60.7 | 61.4 | 0.65 | 39 |
| | | 0.01 | 21.2 | 21.7 | 21.5 | 0.25 | 40 |
| AsCas12a- | HPRT- | 1.00 | 72.8 | 72.2 | 72.5 | 0.30 | 41 |
| wild type | 38115- | 0.22 | 59.6 | 63.0 | 61.3 | 1.70 | 42 |
| | AS | 0.05 | 25.7 | 26.3 | 26.0 | 0.30 | 43 |
| | | 0.01 | 7.9 | 8.9 | 8.4 | 0.50 | 44 |
| Ascas12a- | HPRT- | 1.00 | 75.9 | 76.6 | 76.3 | 0.35 | 45 |
| M537R/ | 38115- | 0.22 | 75.7 | 80.6 | 80.2 | 0.45 | 46 |
| F870L | AS | 0.05 | 77.6 | 79.4 | 78.5 | 0.90 | 47 |
| | | 0.01 | 73.5 | 74.4 | 74.0 | 0.45 | 48 |
| LbCas12a- | HPRT- | 1.00 | 9.7 | 15.4 | 12.6 | 2.85 | 49 |
| wild type | 38146- | 0.22 | 4.1 | 5.0 | 4.6 | 0.45 | 50 |
| | AS | 0.05 | 0.0 | 0.0 | 0.0 | 0.00 | 51 |
| | | 0.01 | 0.0 | 0.0 | 0.0 | 0.00 | 52 |
| LbCas12a- | HPRT- | 1.00 | 70.9 | 68.8 | 69.9 | 1.05 | 53 |
| E795L | 38146- | 0.22 | 50.0 | 38.7 | 44.4 | 5.65 | 54 |
| | AS | 0.05 | 23.8 | 22.7 | 23.2 | 0.55 | 55 |
| | | 0.01 | 9.5 | 7.8 | 8.7 | 0.85 | 56 |
| AsCas12a- | HPRT- | 1.00 | 51.4 | 54.1 | 52.8 | 1.35 | 57 |
| wild type | 38146- | 0.22 | 29.1 | 25.4 | 27.3 | 1.85 | 58 |
| | AS | 0.05 | 10.6 | 11.6 | 11.1 | 0.50 | 59 |
| | | 0.01 | 4.6 | 0.0 | 2.3 | 2.30 | 60 |
| AsCas12a- | HPRT- | 1.00 | 76.2 | 76.5 | 76.4 | 0.15 | 61 |
| M537R/ | 38146- | 0.22 | 62.1 | 62.5 | 62.3 | 0.20 | 62 |
| F870L | AS | 0.05 | 51.7 | 55.3 | 55.3 | 1.80 | 63 |
| | | 0.01 | 32.7 | 33.8 | 33.3 | 0.55 | 64 |
| LbCas12a- | HPRT- | 1.00 | 0.0 | 0.0 | 0.0 | 0.0 | 65 |
| wild type | 38164- | 0.22 | 0.0 | 0.0 | 0.0 | 0.0 | 66 |
| | S | 0.05 | 0.0 | 0.0 | 0.0 | 0.0 | 67 |
| | | 0.01 | 0.0 | 0.0 | 0.0 | 0.0 | 68 |
| LbCas12a- | HPRT- | 1.00 | 7.0 | 8.6 | 7.8 | 0.8 | 69 |
| E795L | 38164- | 0.22 | 0.0 | 0.0 | 0.0 | 0.0 | 70 |
| | S | 0.05 | 0.0 | 0.0 | 0.0 | 0.0 | 71 |
| | | 0.01 | 0.0 | 0.0 | 0.0 | 0.0 | 72 |
| AsCas12a- | HPRT- | 1.00 | 8.2 | 8.0 | 8.1 | 0.10 | 73 |
| wild type | 38164- | 0.22 | 0.0 | 3.8 | 1.9 | 1.90 | 74 |
| | AS | 0.05 | 0.0 | 0.0 | 0.0 | 0.00 | 75 |
| | | 0.01 | 0.0 | 0.0 | 0.0 | 0.00 | 76 |
| AsCas12a- | HPRT- | 1.00 | 68.5 | 69.4 | 69.0 | 0.45 | 77 |
| M537R/ | 38164- | 0.22 | 35.0 | 37.9 | 36.5 | 1.45 | 78 |
| F870L | AS | 0.05 | 32.5 | 31.4 | 32.0 | 0.55 | 79 |
| | | 0.01 | 16.4 | 18.4 | 17.4 | 1 | 80 |
| LbCas12a- | HPRT- | 1.00 | 0.0 | 9.9 | 5.0 | 4.95 | 81 |
| wild type | 38164- | 0.22 | 0.0 | 0.0 | 0.0 | 0.0 | 82 |
| | S | 0.05 | 0.0 | 0.0 | 0.0 | 0.0 | 83 |
| | | 0.01 | 0.0 | 0.0 | 0.0 | 0.0 | 84 |
| LbCas12a- | HPRT- | 1.00 | 30.3 | 27.1 | 28.7 | 1.60 | 85 |
| E795L | 38164- | 0.22 | 8.8 | 7.8 | 8.3 | 5.00 | 86 |
| | S | 0.05 | 4.0 | 6.2 | 5.1 | 1.10 | 87 |
| | | 0.01 | 0.0 | 0.0 | 0.0 | 0.00 | 88 |
| AsCas12a- | HPRT- | 1.00 | 16.1 | 14.0 | 15.1 | 1.05 | 89 |
| wild type | 38164- | 0.22 | 65.0 | 4.9 | 5.7 | 0.80 | 90 |
| | S | 0.05 | 0.0 | 11.3 | 5.7 | 5.65 | 91 |
| | | 0.01 | 0.0 | 0.0 | 0.0 | 0.0 | 92 |
| AsCas12a- | HPRT- | 1.00 | 69.2 | 71.4 | 70.3 | 1.10 | 93 |
| M537R/ | 38164- | 0.22 | 39.0 | 37.9 | 38.5 | 0.55 | 94 |
| F870L | S | 0.05 | 25.8 | 25.3 | 25.6 | 0.25 | 95 |
| | | 0.01 | 11.5 | 11.8 | 11.7 | 0.15 | 96 |
| LbCas12a- | HPRT- | 1.00 | 73.9 | 75.6 | 74.8 | 0.85 | 97 |
| wild type | 38186- | 0.22 | 66.0 | 63.3 | 64.7 | 1.35 | 98 |
| | S | 0.05 | 30.9 | 34.3 | 32.6 | 1.70 | 99 |
| | | 0.01 | 6.7 | 10.3 | 8.5 | 1.80 | 100 |
| LbCas12a- | HPRT- | 1.00 | 74.5 | 72.1 | 73.3 | 1.20 | 101 |
| E795L | 38186- | 0.22 | 75.0 | 73.1 | 74.1 | 0.95 | 102 |
| | S | 0.05 | 50.0 | 50.6 | 50.3 | 0.30 | 103 |
| | | 0.01 | 13.2 | 12.5 | 12.9 | 0.35 | 104 |
| AsCas12a- | HPRT- | 1.00 | 73.4 | 74.1 | 73.8 | 0.35 | 105 |
| wild type | 38186- | 0.22 | 51.2 | 50.8 | 51.0 | 0.50 | 106 |
| | S | 0.05 | 16.7 | 15.7 | 16.2 | 0.50 | 107 |
| | | 0.01 | 4.1 | 4.8 | 4.5 | 0.35 | 108 |
| AsCas12a- | HPRT- | 1.00 | 78.0 | 78.9 | 78.5 | 0.45 | 109 |
| M537R/ | 38186- | 0.22 | 78.6 | 77.1 | 77.9 | 0.75 | 110 |
| F870L | S | 0.05 | 76.9 | 80.2 | 78.6 | 1.65 | 111 |
| | | 0.01 | 62.3 | 64.4 | 63.4 | 1.05 | 112 |
| LbCas12a- | HPRT- | 1.00 | 34.4 | 40.4 | 40.4 | 5.95 | 113 |
| wild type | 38228- | 0.22 | 11.4 | 12.1 | 11.8 | 0.35 | 114 |
| | S | 0.05 | 5.3 | 4.0 | 4.7 | 0.65 | 115 |
| | | 0.01 | 0.0 | 0.0 | 0.0 | 0.00 | 116 |
| LbCas12a- | HPRT- | 1.00 | 67.1 | 66.0 | 66.6 | 55.00 | 117 |
| E795L | 38228- | 0.22 | 34.5 | 33.3 | 33.9 | 0.60 | 118 |
| | S | 0.05 | 8.2 | 7.7 | 8.0 | 0.25 | 119 |
| | | 0.01 | 0.0 | 3.0 | 1.5 | 1.50 | 120 |
| AsCas12a- | HPRT- | 1.00 | 46.5 | 48.4 | 47.5 | 0.95 | 121 |
| wild type | 38228- | 0.22 | 23.8 | 24.7 | 24.3 | 0.45 | 122 |
| | S | 0.05 | 7.9 | 6.7 | 7.3 | 0.60 | 123 |
| | | 0.01 | 4.3 | 3.8 | 4.1 | 0.25 | 124 |
| AsCas12a- | HPRT- | 1.00 | 76.7 | 78.8 | 77.8 | 1.05 | 125 |
| M537R/ | 38228- | 0.22 | 67.3 | 67.9 | 67.6 | 0.30 | 126 |
| F870L | S | 0.05 | 60.9 | 60.7 | 60.8 | 0.10 | 127 |
| | | 0.01 | 30.1 | 32.6 | 31.4 | 1.25 | 128 |
| LbCas12a- | HPRT- | 1.00 | 81.3 | 79.8 | 80.6 | 0.75 | 129 |
| wild type | 38330- | 0.22 | 61.1 | 62.3 | 61.7 | 0.60 | 130 |
| | AS | 0.05 | 13.2 | 14.1 | 13.7 | 0.45 | 131 |
| | | 0.01 | 4.7 | 5.0 | 4.9 | 0.15 | 132 |
| LbCas12a- | HPRT- | 1.00 | 58.9 | 58.4 | 58.7 | 0.25 | 133 |
| E795L | 38330- | 0.22 | 71.3 | 71.4 | 71.4 | 0.05 | 134 |
| | AS | 0.05 | 35.4 | 41.8 | 38.6 | 3.20 | 135 |
| | | 0.01 | 7.0 | 6.8 | 6.9 | 0.10 | 136 |
| AsCas12a- | HPRT- | 1.00 | — | 79.4 | 79.4 | 0.00 | 137 |
| wild type | 38330- | 0.22 | 79.8 | 77.8 | 78.8 | 1.00 | 138 |
| | AS | 0.05 | 49.4 | 50.0 | 49.7 | 0.30 | 139 |
| | | 0.01 | 17.3 | 15.7 | 16.5 | 0.80 | 140 |
| AsCas12a- | HPRT- | 1.00 | 79.2 | 66.7 | 73.0 | 6.25 | 141 |
| M537R/ | 38330- | 0.22 | 83.7 | 83.2 | 83.5 | 0.25 | 142 |
| F870L | AS | 0.05 | 77.4 | 76.1 | 76.8 | 0.65 | 143 |
| | | 0.01 | 26.2 | 27.2 | 26.7 | 0.50 | 144 |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but no limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention, and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The term “wild-type LbCas12a” (“wild-type Lb enzyme” or “WT-LbCas12a”) encompasses a protein having the identical amino acid sequence of the naturally-occurring Lachnospiraceae bacterium ND2006 Cas12a (e.g., SEQ ID NO: 2) and that has biochemical and biological activity when combined with a suitable crRNA to form and active CRISPR/Cas12a endonuclease system. The term “wild-type AsCas12a” (“wild-type as enzyme” or “WT-AsCas12a”) encompasses a protein having the identical amino acid sequence of the naturally-occurring Acidaminococcus sp. BV3L6 Cas12a (e.g., SEQ ID NO: 18) and that has biochemical and biological activity when combined with a suitable crRNA to form and active CRISPR/Cas12a endonuclease system.

The term “mutant LbCas12a protein” encompasses protein forms having a different amino acid sequence form the wild-type Lachnospiraceae bacterium ND2006 Cas12a and that have biochemical and biological activity with combined with a suitable crRNA to form an active CRISPR-Cas12a endonuclease system. This includes orthologs and Cas12a variants having different amino acid sequences form the wild-type Lachnospiraceae bacterium ND2006 Cas12a.

The term “polypeptide” refers to any linear or branched peptide comprising more than one amino acid. Polypeptide includes protein or fragment thereof or fusion thereof, provided such protein, fragment or fusion retains a useful biochemical or biological activity.

Fusion proteins typically include extra amino acid information that is not native to the protein to which the extra amino acid information is covalently attached. Such extra amino acid information may include tags that enable purification or identification of the fusion protein. Such extra amino acid information may include peptides that enable the fusion proteins to be transported into cells and/or transported to specific locations within cells. Examples of tags for these purposes include the following: AviTag, which is a peptide allowing biotinylation by the enzyme BirA so the protein can be isolated by streptavidin (GLNDIFEAQKIEWHE) (SEQ ID NO: 53); Calmodulin-tag, which is a peptide bound by the protein calmodulin (KRRWKKNFIAVSAANRFKKISSSGAL) (SEQ ID NO: 54); polyglutamate tag, which is a peptide binding efficiently to anion-exchange resin such as Mono-Q (EEEEEE) (SEQ ID NO: 55); E-tag, which is a peptide recognized by an antibody (GAPVPYPDPLEPR) (SEQ ID NO: 56); FLAG-tag, which is a peptide recognized by an antibody (DYKDDDDK) (SEQ ID NO: 57); HA-tag, which is a peptide from hemagglutinin recognized by an antibody (YPYDVPDYA) (SEQ ID NO: 58); His-tag, which is typically 5-10 histidines bound by a nickel or cobalt chelate (HHHHHH) (SEQ ID NO: 59); Myc-tag, which is a peptide derived from c-myc recognized by an antibody (EQKLISEEDL) (SEQ ID NO: 60); NE-tag, which is a novel 18-amino-acid synthetic peptide (TKENPRSNQEESYDDNES) (SEQ ID NO: 61) recognized by a monoclonal IgG1 antibody, which is useful in a wide spectrum of applications including Western blotting, ELISA, flow cytometry, immunocytochemistry, immunoprecipitation, and affinity purification of recombinant proteins; S-tag, which is a peptide derived from Ribonuclease A (KETAAAKFERQHMDS) (SEQ ID NO: 62); SBP-tag, which a peptide which binds is to streptavidin; (MDEKTTGWRGGHVVEGLAGELEQLRARLEHHPQGQREP) (SEQ ID NO: 63); Softag 1, which is intended for mammalian expression (SLAELLNAGLGGS) (SEQ ID NO: 64); Softag 3, which is intended for prokaryotic expression (TQDPSRVG) (SEQ ID NO: 65); Strep-tag, which is a peptide which binds to streptavidin or the modified streptavidin called streptactin (Strep-tag II: WSHPQFEK) (SEQ ID NO: 66); TC tag, which is a tetracysteine tag that is recognized by FIAsH and ReAsH biarsenical compounds (CCPGCC) (SEQ ID NO: 67); V5 tag, which is a peptide recognized by an antibody (GKPIPNPLLGLDST) (SEQ ID NO: 68); VSV-tag, a peptide recognized by an antibody (YTDIEMNRLGK) (SEQ ID NO: 69); Xpress tag (DLYDDDDK) (SEQ ID NO: 70); Isopeptag, which is a peptide which binds covalently to pilin-C protein (TDKDMTITFTNKKDAE) (SEQ ID NO: 71); SpyTag, which is a peptide which binds covalently to SpyCatcher protein (AHIVMVDAYKPTK) (SEQ ID NO: 72); SnoopTag, a peptide which binds covalently to SnoopCatcher protein (KLGDIEFIKVNK) (SEQ ID NO: 73); BCCP (Biotin Carboxyl Carrier Protein), which is a protein domain biotinylated by BirA to enable recognition by streptavidin; Glutathione-S-transferase-tag, which is a protein that binds to immobilized glutathione; Green fluorescent protein-tag, which is a protein which is spontaneously fluorescent and can be bound by antibodies; HaloTag, which is a mutated bacterial haloalkane dehalogenase that covalently attaches to a reactive haloalkane substrate to allow attachment to a wide variety of substrates; Maltose binding protein-tag, a protein which binds to amylose agarose; Nustag; Thioredoxin-tag; and Fc-tag, derived from immunoglobulin Fc domain, which allows dimerization and solubilization and can be used for purification on Protein-A Sepharose. Nuclear localization signals (NLS), such as those obtained from SV40, allow for proteins to be transported to the nucleus immediately upon entering the cell. Given that the native Cas9 protein is bacterial in origin and therefore does not naturally comprise a NLS motif, addition of one or more NLS motifs to the recombinant Cas9 protein is expected to show improved genome editing activity when used in eukaryotic cells where the target genomic DNA substrate resides in the nucleus. One skilled in the art would appreciate these various fusion tag technologies, as well as how to make and use fusion proteins that include them.

### REFERENCES

1. Zetsche et al., “Cpf1 Is a Single RNA-Guided Endonuclease of a Class 2 CRISPR-Cas System,” Cell 163: 759-771 (2015).
2. Hur et al., “Targeted mutagenesis in mice by electroporation of Cpf1 ribonucleoproteins,” Nature Biotech. 34(8): 807-808 (2016).
3. Kim et al., “Generation of knockdown mice by Cpf1-mediated gene targeting,” Nature Biotech. 34(8): 808-810 (2016).
4. Kim et al., “Genome-wide analysis reveals specificities of Cpf1 endonucleases in human cells,” Nature Biotech. 34(8): 863-868 (2016).
5. Kleinstiver et al., “Genome-wide specificities of CRISPR-Cas Cpf1 nucleases in human cells,” Nature Biotech. 34(8): 869-874 (2016).
6. Kim et al., “In vivo high-throughput profiling of CRISPR-Cpf1 activity,” Nature Methods 14(2): 153-159 (2017).
7. Zetsche et al., “Multiplex gene editing by CRISPR-Cpf1 using a single rRNA array,” Nature Biotech. 35(1): 31-34 (2017).
8. Kim et al., “CRISPR/Cpf1-mediated DNA-free plant genome editing,” Nature Comm. 8(14406) (2017)
9. Yamano et al., “Crystal Structure of Cpf1 in Complex with Guide RNA and Target RNA. Cell 65: 949-962 (2016).
10. Yamano et al., “Structural Basis for the Canonical and Non-canonical PAM Recognition by CRISPR-Cpf1,” Mol. Cell 67: 633-645 (2017).
11. Gao, L., et al., “Engineered Cpf1 variants with altered PAM specificities,” Nature Biotech. 35(8): 789-792 (2017).
12. Robert and Gouet, “Deciphering key features in protein structures with the new ENDscript server,” Nucl. Acids Res. 42(W1): W320-W324 (2014).

What is claimed:

1. An isolated mutant LbCas12a comprising a substitution mutation selected from the group consisting of
(a) a single substitution mutation introduced into the wild-type LbCas12a protein selected from the following positions N527, D559, and E795: or
(b) a multiple substitution mutation introduced into the wild-type LbCas12a protein selected from at least two of the following positions: N527, D559, and E795.

2. The isolated mutant LbCas12a protein of claim 1, wherein the isolated mutant LbCas12a protein is selected form the group consisting of SEQ ID NO: 3, SEQ ID NO: 4, and SEQ ID NO: 5.

3. The isolated mutant LbCas12a protein of claim 1, wherein the isolated mutant LbCas12a protein is selected from the group consisting of SEQ ID NO: 6, SEQ ID NO: 7, SEQ ID NO: 8, and SEQ ID NO: 9.

4. An isolated ribonucleoprotein complex, comprising:
(a) the mutant LbCas12a protein of claim 1; and
(b) a gRNA complex,
wherein the isolated ribonucleoprotein complex is active as a CRISPR/Cas12a endonuclease system, wherein the resultant CRISPR/Cas12a endonuclease system displays maintained on-target editing activity relative to a wild-type CRISPR/Cas12a endonuclease system.

6. The isolated ribonucleoprotein complex of claim 4, wherein the mutant LbCas12a protein is selected from the group consisting of SEQ ID NO: 3, SEQ ID NO: 4, SEQ ID NO: 5, SEQ ID NO: 6, SEQ ID NO:7, SEQ ID NO: 8, and SEQ ID NO: 9.

6. The CRISPR/Cas12a endonuclease system comprising a mutant LbCas12a protein and a gRNA, wherein the CRISPR/Cas12a endonuclease system displays maintained on-target editing activity relative to a wild-type CRISPR/Cas12a endonuclease system.

7. The CRISPR/Cas12a endonuclease system of claim 6, wherein the CRISPR/Cas12a endonuclease system is encoded by a DNA expression vector.

8. The CRISPR/Cas12a endonuclease system of claim 7, wherein the DNA expression vector comprises a plasmid-borne vector.

9. The CRISPR/Cas12a endonuclease system of claim 8, wherein the DNA expression vector is selected form a bacterial expression vector and a eukaryotic expression vector.

10. An isolated nucleic acid encoding a mutant LbCas12a protein, wherein the mutant LbCas12a protein is active in CRISPR/Cas12a endonuclease system, wherein the CRISPR/Cas12a endonuclease system displays maintained on-target editing activity relative to a wild-type CRISPR/Cas12a endonuclease system.

11. The isolated nucleic acid encoding a mutant LbCas12a protein of claim 10, wherein the mutant LbCas12a protein comprises a substitution mutation selected from the group consisting of
(a) a single substitution mutation introduced into the wild-type Cas12a protein selected from the following positions: N527, D559, and E795; or a multiple substitution mutation introduced into the wild-type Cas12a protein selected from at least two of the following positions: N527, D559, and E795.

12. The isolated nucleic acid encoding a mutant Cas12a protein of claim 10, wherein the mutant Cas12a protein is selected from the group consisting of SEQ ID NO: 10, SEQ ID NO: 11, SEQ ID NO: 12, SEQ ID NO: 14, SEQ ID NO: 14, SEQ ID NO: 15, and SEQ ID NO: 16.
